(19) United States
(12) Patent Application Publication
(10) Pub. No.: US 2006/0079674 A1
(43) Pub. Date: Apr. 13, 2006

(54) Contraceptive targets

(75) Inventors: Martin M. Matzuk, Pearland, TX (US); Yuchen Bai, Newtown, PA (US); Pei Wang, Houston, TX (US); Xuemei Wu, Dallas, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine Wyeth

(21) Appl. No.: 10/965,103

(22) Filed: Oct. 14, 2004

Related U.S. Application Data
(63) Continuation of application No. PCT/US03/12720, filed on Apr. 23, 2003.
(63) Continuation-in-part of application No. PCT/US02/13245, filed on Apr. 26, 2002.
Application No. PCT/US02/13245 is a continuation-in-part of application No. 09/844,864, filed on Apr. 27, 2001, now abandoned.
Application No. 09/844,864 is a continuation-in-part of application No. PCT/US99/25209, filed on Oct. 28, 1999.
(60) Provisional application No. 60/411,262, filed on Sep. 17, 2002.
(60) Provisional application No. 60/434,165, filed on Dec. 17, 2002.
(60) Provisional application No. 60/439,781, filed on Jan. 13, 2003.
(60) Provisional application No. 60/442,164, filed on Jan. 23, 2003.
(60) Provisional application No. 60/106,020, filed on Oct. 28, 1998.

(51) Int. Cl. C07K 14/575; C07K 16/26; C07H 21/02; C12P 21/06

(52) U.S. Cl. 536/23.1; 530/388.24; 530/399; 435/69.1; 435/320.1; 435/325

(57) ABSTRACT

The present invention relates generally to ovary-specific genes (O1-180, O1-184 and O1-236) and the proteins they encode. Also provided are methods for detecting cell proliferative or degenerative disorders in reproductive tissues. Yet further, the invention provides methods for scrounge of compounds that interact and/or modulate the expression or activity of the ovary-specific genes. These compounds are possible contraceptive agents and/or fertility agents.

This application is a continuation application of International Application Number PCT/US03/12720 filed Apr. 23, 2003, which is a continuation-in-part of International Application Number PCT/US02/13245, filed on Apr. 26, 2002 and claims priority to U.S. Provisional Application No. 60/442,164 filed on Jan. 23, 2003, U.S. Provisional Application No. 60/439,781, which was filed on Jan. 13, 2003; U.S. Provisional Application No. 60/434,165, which was filed on Dec. 17, 2002 and U.S. Provisional Application No. 60/411,262 filed Sep. 17, 2002.

### BACKGROUND OF THE INVENTION

A. Field of the Invention

The present invention relates generally to ovary-specific genes and the proteins they encode.

B. Description of Related Art

Reproductive development and function are complex processes involving both genetically-determined and physiological events. Identification of the critical protein products of genes involved in these processes is necessary to characterize how these processes are regulated. Although important molecular events occur during the early phases of mammalian oogenesis and folliculogenesis, to date, few “candidate” regulatory molecules have been identified and characterized thoroughly. Several studies have suggested that both endocrine factors, such luteinizing hormone (LH) and follicle stimulating hormone (FSH) from the pituitary, as well as paracrine factors secreted from the oocyte influence folliculogenesis. FSH and LH are known to bind to granulosa and thecal cells which in turn are required for oocyte growth and maturation and maintenance of oocyte meiotic competence. Likewise, oocytes may secrete factors which are necessary for normal granulosa cell and thecal cell function. Because oocyte growth is coordinated with the development and growth of the surrounding somatic cells (i.e., granulosa cells initially and thecal cells later), understanding the molecular events at early stages will give important clues about the paracrine factors mediating the reciprocal interactions between oocytes and somatic cells, the development of competence for trophic hormone stimulation, the process of follicular recruitment, and the regulation of the ovulation process.

Disruption of the hypothalamic-pituitary-gonadal reproductive axis by administration of steroids containing synthetic estrogens and progestins has been one of the oldest methods of hormonal contraception. However, the latest report of the Institute of Medicine emphasizes the importance of developing strategies for new contraceptives. According to the report, some of the long-term contraceptive strategies for women include inhibition of ovulation, prevention of fertilization, or blocking of implantation of a fertilized egg into the uterine lining. Furthermore, infertility affects ˜15% of couples, and in ˜40% of the cases, the female is believed to be the sole cause of the infertility. Thus, it is critical to identify novel ovary-specific gene products which could be potential targets for new contraceptive agents as well as determining the etiology of specific forms of female infertility.

One function of the ovary is to produce an oocyte that is fully capable of supplying all the necessary proteins and factors for fertilization and early embryonic development. Oocyte-derived mRNA and proteins are necessary for the removal of the sperm nuclear envelope, the decondensation of the sperm nucleus (including the removal of protamines), the assembly of histones on the sperm DNA and chromatin condensation, the completion of oocyte meiotic maturation and extrusion of the second polar body, the formation of male and female pronuclei, the fusion of male and female pronuclei, the replication of DNA, and the initiation of zygote and early embryonic cleavages [reviewed in (Perreault, 1992)]. Oocyte-derived factors are necessary since the sperm contains mainly DNA (i.e., no cytoplasm or nucleoplasm), and many of the factors necessary for early post-fertilization events in mammals are acquired during oocyte meiotic maturation (McLay and Clarke, 1997). These oocyte proteins are predicted to be highly conserved through evolution since oocytes can efficiently remodel heterologous sperm or somatic cell nuclei into pronuclei (Perreault, 1992). Although histones are involved in the modification of the sperm chromatin to resemble that of a somatic cell, the other non-histone proteins involved in these processes are unknown in mammals. In Xenopus laevis, a key factor in sperm decondensation is nucleoplasmin which was isolated and cloned over a decade ago (Burglin et al., 1987; Dingwall et al., 1987). Sperm chromatin decondensation occurs after a spermatotozoon enters an egg. In Xenopus laevis, although reduction of the protamine disulfide bonds by ooplasmic glutathione is important, nucleoplasmin (also called nucleoplasmin A or Xnpm2) is necessary and sufficient to initiate the decondensation of sperm nuclei (Philpott et al., 1991). Nucleoplasmin, an acidic, thermostable protein, is the most abundant protein in the nucleus of Xenopus laevis oocytes and eggs, making up 7-10% of the total nuclear protein (Krohne and Franke, 1980a; Mills et al., 1980). After germinal vesicle breakdown, nucleoplasmin [present in the egg nucleoplasm but not bound to DNA (Mills et al., 1980)], is released into the ooplasm where it functions to bind protamines tightly and strip them from the sperm nucleus within 5 minutes of sperm entry, resulting in sperm decondensation (Ohsumi and Katagiri, 1991; Philpott and Leno, 1992; Philpott et al., 1991). This process allows egg histones to subsequently bind the sperm DNA. Immunodepletion of nucleoplasmin from egg extracts prevents sperm decondensation (Philpott et al., 1991). Direct interaction of nucleoplasmin with protamine was observed in in vitro experiments. The data suggest that the nucleoplasmin is bound to protamine in a 1:1 ratio and that the polyglutamic acid tract in nucleoplasmin plays a critical role for binding to protamine (Iwata et al., 1999). Interestingly, injection of sperm DNA into oocyte nuclei, male or female pronuclei of fertilized eggs, or nuclei of 2 cell embryos leads to sperm decondensation (Maeda et al., 1998), suggesting that nucleoplasmin is functional at all of these stages. Nucleoplasmin can also interact with histones as a pentamer (Earnshaw et al., 1980; Laskey et al., 1993). Nucleoplasmin binds specifically to histones H2A and H2B and along with the proteins N1/N2 that bind histones H3 and H4, can promote nucleosome assembly onto DNA (Dilworth et al., 1987; Laskey et al., 1993). These observations suggest that during oogenesis and during oogenesis and at fertilization, the oocyte-derived nucleoplasmin interacts with the female pronucleus and male pronucleus, interacts with histones, and is required in some way for chromatin assembly. (Laskey et al., 1993; Philpott et al., 1991). Although “ubiquitous” proteins with low homology to nucleoplasmin have been cloned in mammals and Drosophila (Chan et al., 1989; Crevel et al., 1997; Ito et al., 1996; MacArthur and Shackleford, 1997b; Schmidt-Zachmann and Franke, 1988), an oocyte-equivalent ortholog in mammals had not yet been identified.

The basic functional unit within the ovary is the follicle, which consists of the oocyte and its surrounding somatic cells. Fertility in female mammals depends on the ability of the ovaries to produce Graafian (pre-ovulatory) (pre-ovulatory) follicles, which ovulate fertilizable oocytes at mid-cycle (Erickson and Shimasaki, 2000). This process, termed folliculogenesis, requires a precise coordinate regulation between extraovarian and intraovarian factors (Richards, et al., 1995). Compared to the knowledge of extraovarian regulatory hormones at the levels of the hypothalamus (i.e., GnRH) and anterior pituitary (i.e., FSH and LH), little is known about paracrine and autocrine factors within the ovaries, though oocyte-somatic cell communication has been long recognized as important (Falck, 1959). Accumulating evidence shows that factors secreted by the oocyte promote the proliferation of surrounding granulosa cells, and inhibit premature luteinization of these cells during folliculogenesis (El-Fouly et al., 1970; Channing, 1970). Oocyte factors have been implicated in controlling granulosa cell synthesis of hyaluronic acid, urokinase plasminogen activator (uPA), LH receptor, steroids and prostaglandins and prostaglandins (El-Fouly et al., 1970; Nekola and Nalbandov, 1971; Salustri et al., 1985; Vanderhyden et al., 1993; Eppig et al., 1997a, b).

Several novel regulatory proteins have been recently discovered within oocytes. Growth differentiation factor 9 (GDF-9 or Gdf9), a member of transforming growth factor β (TGF-β) superfamily, is one of the most important signaling factors. Oocyte expression of GDF-9 begins at the primary follicle stage, and persists through ovulation in the mouse (McGrath et al., 1995; Elvin et al., 2000). Female Gdf9 knockout mice are infertile due to a block of folliculogenesis at the type 3b (primary) follicle stage, accompanied by defects in granulosa cell growth and differentiation, theca cell formation, and oocyte meiotic competence (Dong et al., 1996; Carabatsos et al., 1998, Elvin et al, 1999A). Also, recombinant GDF-9 affects the expression of the genes encoding hyaluronan synthase 2 (Has2), cyclooxygenase 2 (Cox2), steroid acute regulatory protein (StAR), the prostaglandin E2 receptor EP2, pentraxin 3, the LH receptor and uPA (Elvin et al., 1999B, Elvin et al., 2000).

To identify key proteins in the hypothalamic-pituitary-gonadal axis, several important knockout mouse models have been generated, including four which have ovarian defects. Mice lacking the gonadal/pituitary peptide inhibin have secondary infertility due to the onset of ovarian or testicular tumors which appear as early as 4 weeks of age (Matzuk et al., 1992). Mice lacking activin receptor type II (Acvr2) survive to adulthood but display reproductive defects. Male mice show reduced testes size and demonstrate delayed fertility (Matzuk, et al. 1995). In contrast, female mice have a block in folliculogenesis at the early antral follicle stage leading to infertility. Consistent with the known role of activins in FSH homeostasis, both pituitary and serum FSH levels are dramatically reduced in these Acvr2 knockout mice. Female mice lacking FSH, due to a mutation in the FSHbeta gene, are infertile (Kumar et al., 1997). However, these mice have an earlier block in folliculogenesis prior to antral follicle formation. Thus, FSH is not required for formation of a multi-layer pre-antral follicle, but it is required for progression to antral follicle formation. Finally, growth differentiation factor 9 (Gdf9) knockout mice have been used to determine at which stage in follicular development GDF-9 is required (Dong et al., 1996). Within the ovary, expression of Gdf9 mRNA is limited to the oocyte and is seen at the early one-layer primary follicle stage and persists through ovulation. Absence of GDF-9 results in ovaries that fail to demonstrate any normal follicles beyond the primary follicle stage. Although oocytes surrounded by a single layer of granulosa cells are present and appear normal histologically, no normal two-layered follicles are present. Follicles beyond the one-layer stage are abnormal, contain atypical granulosa cells, and display asymmetric growth of these cells. Furthermore, as determined by light and electron microscopy, a thecal cell layer does not form in these Gdf9 knockout ovaries (Dong et al., 1996; Elvin et al., 1999). Thus, in contrast to kit ligand and other growth factors which are synthesized by the somatic cells and influence oocyte growth, GDF-9 functions in the reciprocal manner as an oocyte-derived growth factor which is required for somatic cell function.

### BRIEF SUMMARY OF THE INVENTION

The present invention provides three ovary-specific and oocyte-specific polynucleotide sequences, O1-180 (also known as zygote arrest 1 (Zar1)) (SEQ.ID.NO.1, SEQ.ID.NO.11, SEQ.ID.NO.12, SEQ.ID.NO.13, SEQ.ID.NO.28, SEQ.ID.NO.30, SEQ.ID.NO.31, SEQ.ID.NO.33, SEQ.ID.NO.35, SEQ.ID.NO.37, SEQ.ID.NO.38, SEQ.ID.NO.40 and SEQ.ID.NO.41), O1-184 (SEQ.ID.NO.3) and O1-236 (also known as nucleoplasmin (Npm2)) (SEQ.ID.NO.5, SEQ.ID.NO.7, SEQ.ID.NO.8; SEQ.ID.NO.10, SEQ.ID.NO.14 and SEQ.ID.NO.43), the protein products they encode, fragments, homologues, and derivatives thereof, and antibodies which are immunoreactive with these protein products. These genes and their protein products appear to relate to various cell proliferative or degenerative disorders, especially those involving ovarian tumors, such as germ cell tumors and granulosa cell tumors, or infertility, such as premature ovarian failure.

In a specific embodiment, the present invention provides nucleic acid molecules that are specific to gonadal tissue. These specific nucleic acids may be a naturally-occurring cDNA, genomic DNA, RNA, or a fragment of one of these nucleic acids, or may be a non-naturally-occurring nucleic acid molecule. If the specific nucleic acid is genomic DNA, then it is a gonadal specific gene. In one embodiment, the nucleic acid molecule encodes a polypeptide that is specific to the gonads. In another preferred embodiment, the nucleic acid molecule encodes a polypeptide that comprises an amino acid sequence of O1-180 (SEQ.ID.NO.2, SEQ.ID.NO.16, SEQ.ID.NO.29, SEQ.ID.NO.32, SEQ.ID.NO.34, SEQ.ID.NO.36 and SEQ.ID.NO.39), O1-184 (SEQ.ID.NO.4), O1-236 (SEQ.ID.NO.6, SEQ.ID.NO.9, and SEQ.ID.NO.42). In yet another, the nucleic acid molecule comprises a nucleic acid sequence of O1-180 (also known as zygote arrest 1 (Zar1)) (SEQ.ID.NO.1, SEQ.ID.NO.11, SEQ.ID.NO.12, SEQ.ID.NO.13, SEQ.ID.NO.28, SEQ.ID.NO.30, SEQ.ID.NO.31, SEQ.ID.NO.33, SEQ.ID.NO.35, SEQ.ID.NO.37, SEQ.ID.NO.38, SEQ.ID.NO.40 and SEQ.ID.NO.41), O1-184 (SEQ.ID.NO.3) and O1-236 (also known as nucleoplasmin (Npm2)) (SEQ.ID.NO.5, SEQ.ID.NO.7, SEQ.ID.NO.8; SEQ.ID.NO.10, SEQ.ID.NO.14 and SEQ.ID.NO.43). By nucleic acid molecule, it is also meant to be inclusive of sequences that selectively hybridize or exhibit substantial sequence similarity to a nucleic acid molecule encoding a gonadal specific protein, or that selectively hybridize or exhibit substantial sequence similarity to a gonadal specific nucleic acids, as well as allelic variants of a nucleic acid molecule encoding a gonadal specific protein, and allelic variants of a gonadal specific nucleic acids. Nucleic acid molecules comprising a part of a nucleic acid sequence that encodes a gonadal specific protein or that comprises a part of a nucleic acid sequence of gonadal specific nucleic acids are also provided.

Thus, in one embodiment, the invention provides methods for detecting cell proliferative or degenerative disorders of ovarian origin and which are associated with O1-180, O1-184 or O1-236. In another embodiment, the invention provides method of treating cell proliferative or degenerative disorders associated with abnormal levels of expression of O1-180, O1-184 or O1-236, by suppressing or enhancing their respective activities.

In a specific embodiment, the present invention provides a pharmaceutical composition comprising a modulator of O1-180, O1-184 and/or O1-236 expression dispersed in a pharmaceutically acceptable carrier. The modulator may suppress or enhance transcription of an O1-180, O1-184 and/or O1-236 gene. The modulator may be a polypeptide sequence, a protein, a small molecule, or a polynucleotide sequence. Specifically, the polynucleotide sequence is DNA or RNA. In further embodiments, the polynucleotide sequence is comprised in an expression vector operatively linked to a promoter.

A further embodiment of the present invention is a pharmaceutical composition comprising a modulator of O1-180, O1-184 and/or O1-236 activity dispersed in a pharmaceutically acceptable carrier. The composition may inhibit or stimulate O1-180, O1-184 and/or O1-236 activity. The composition may be a protein, polypeptide sequence, small molecule, or polynucleotide sequence. It is envisioned that the composition may block or enhance the interaction of the nucleic acid sequences in question with the other protein partners.

Another embodiment of the present invention is a method of modulating contraception comprising administering to an animal an effective amount of a modulator of O1-180, O1-184 and/or O1-236 activity and/or expression dispersed in a pharmacologically acceptable carrier, wherein said amount is capable of decreasing conception. The animal may be a male or a female.

A further embodiment is a method of enhancing fertility comprising administering to an animal an effective amount of a modulator of O1-180, O1-184 and/or O1-236 activity and/or expression dispersed in a pharmacologically acceptable carrier, wherein said amount is capable of increasing conception.

Yet further, another embodiment is a method of screening for a modulator of O1-180, O1-184 and/or O1-236 expression comprising the steps of: providing a cell expressing an O1-180, O1-184 and/or O1-236 polypeptide; contacting said cell with a candidate modulator; measuring O1-180, O1-184 and/or O1-236 expression; and comparing the O1-180, O1-184 and/or O1-236 expression in the presence of the candidate modulator with the expression of O1-180, O1-184 and/or O1-236 in the absence of the candidate modulator; wherein a difference in the expression of O1-180, O1-184 and/or O1-236 in the presence of the candidate modulator, as compared with the expression of O1-180, O1-184 and/or O1-236 in the absence of the candidate modulator, identifies the candidate modulator as a modulator of O1-180, O1-184 and/or O1-236 expression.

A specific embodiment of the present invention is a method of identifying compounds that modulate the activity of O1-180, O1-184 and/or O1-236 comprising the steps of obtaining an isolated O1-180, O1-184 and/or O1-236 polypeptide or functional equivalent thereof; admixing the O1-180, O1-184 and/or O1-236 polypeptide or functional equivalent thereof with a candidate compound; and measuring an effect of said candidate compound on the activity of O1-180, O1-184 and/or O1-236.

Another embodiment is method of screening for a compound which modulates the activity of O1-180, O1-184 and/or O1-236 comprising exposing O1-180, O1-184 and/or O1-236 or a O1-180, O1-184 and/or O1-236 binding fragment thereof to a candidate compound; and determining whether said compound binds to O1-180, O1-184 and/or O1-236 or the O1-180, O1-184 and/or O1-236 binding partner thereof; and further determining whether said compound modulates O1-180 or the O1-180 interaction with a binding partner.

Yet further, another embodiment is a method of screening for an interactive compound which binds with O1-180, O1-184 and/or O1-236 comprising exposing a O1-180, O1-184 and/or O1-236 protein, or a fragment thereof to a compound; and determining whether said compound bound to the O1-180, O1-184 and/or O1-236.

Another embodiment is a method of identifying a compound that effects O1-180; O1-184 and/or O1-236 activity. The method comprises the steps of providing a group of transgenic animals having (1) a regulatable one or more O1-180, O1-184 and/or O1-236 protein/genes, (2) a knock-out of one or more O1-180, O1-184 and/or O1-236 protein/genes, or (3) a knock-in of one or more O1-180, O1-184 and/or O1-236 protein/genes; providing a second group of control animals respectively for the group of transgenic animals; and exposing the transgenic animal group and control animal group to a potential O1-180, O1-184 and/or O1-236-modulating compounds; and comparing the transgenic animal group and the control animal group and determining the effect of the compound on one or more proteins related to infertility or fertility in the transgenic animals as compared to the control animals.

In specific embodiments, the present invention provides a method of detecting a binding interaction of a first peptide and a second peptide of a peptide binding pair, comprising culturing at least one eukaryotic cell under conditions suitable to detect the selected phenotype; wherein the cell comprises; a nucleotide sequence encoding a first heterologous fusion protein comprising the first peptide or a segment thereof joined to a transcriptional activation protein DNA binding domain; a nucleotide sequence encoding a second heterologous fusion protein comprising the second peptide or a segment thereof joined to a transcriptional activation protein of a transcriptional activation domain; wherein binding of the first peptide or segment thereof and the second peptide or segment thereof reconstitutes a transcriptional activation protein; and a reporter element activated under positive transcriptional control of the reconstituted transcriptional activation protein, wherein expression of the reporter element produces a selected phenotype; detecting the binding interaction of the peptide binding pair by determining the level of the expression of the reporter element which produces the selected phenotype; wherein said first or second peptide is an O1-180, O1-184 and/or O1-236 peptide and the other peptide is a test peptide, preferably selected peptides/proteins present in a reproductive tissue. In specific embodiments the reproductive tissue is an ovary or testis.

A further embodiment is a rescue screen for detecting the binding interaction of a first peptide and a second peptide of a peptide binding pair. The screen comprises the steps of culturing at least one eukaryotic cell under conditions to detect a selected phenotype or the absence of such phenotype, wherein the cell comprises; a nucleotide sequence encoding a first heterologous fusion protein comprising the first peptide or a segment thereof joined to a DNA binding domain of a transcriptional activation protein; a nucleotide sequence encoding a second heterologous fusion protein comprising the second peptide or a segment thereof joined to a transcriptional activation domain of a transcriptional activation protein; wherein binding of the first peptide or segment thereof and the second peptide or segment thereof reconstitutes a transcriptional activation protein; and a reporter element activated under positive transcriptional control of the reconstituted transcriptional activation protein, wherein expression of the reporter element prevents exhibition of a selected phenotype; detecting the ability of the test peptide to interact with O1-180, O1-184 and/or O1-236 by determining whether the test peptide affects the expression of the reporter element which prevents exhibition of the selected phenotype, wherein said first or second peptide is an O1-180, O1-184 and/or O1-236 peptide and the other peptide is a test peptide, preferably selected peptides/proteins present in a reproductive tissue. In specific embodiments, the reproductive tissue is an ovary, testis, epididymis, vas deferens, etc.

Yet further, another embodiment is a method of identifying binding partners for O1-180, O1-184 and/or O1-236 comprising the steps of: exposing the protein to a potential binding partner; and determining if the potential binding partner binds to O1-180, O1-184 and/or O1-236.

The present invention provides key in vitro and in vivo reagents for studying ovarian development and function. The possible applications of these reagents are far-reaching, and are expected to range from use as tools in the study of development to therapeutic reagents against cancer. The major application of these novel ovarian gene products is to use them as reagents to evaluate and/or develop potential contraceptives to modulate ovulation in women in a reversible or irreversible manner. It will also be expected that these novel ovarian gene products will be useful to screen for genetic mutations in components of those signaling pathways that are associated with some forms of human infertility or gynecological cancers. In addition, depending on the phenotypes of humans with mutations in these genes or signaling pathways, the inventors may consider using these novel ovarian gene products as reagent tools to generate a number of mutant mice for the further study of oogenesis, folliculogenesis, and/or early embryogenesis as maternal effect genes. Such knockout mouse models will provide key insights into the roles of these gene products in human female reproduction and permit the use of these gene products as practical reagents for evaluation and development of new contraceptives.

Still further, another embodiment of the present invention comprises a method of treating an animal suffering from infertility by screening for a modulator that modulates the activity and/or expression of O1-180, O1-184 and/or O1-236 comprising the steps of obtaining an isolated O1-180, O1-184 and/or O1-236 polypeptide or functional equivalent thereof; admixing the O1-180, O1-184 and/or O1-236 polypeptide or functional equivalent thereof with a candidate compound; measuring an effect of said candidate compound on the activity and/or expression of O1-180, O1-184 and/or O1-236, and administering to the subject an effective amount of the modulator to increase conception.

Still further, another embodiment of the present invention comprises a method of modulating conception or fertility in an animal by screening for a modulator that modulates the activity and/or expression of O1-180, O1-184 and/or O1-236 comprising the steps of obtaining an isolated O1-180, O1-184 and/or O1-236 polypeptide or functional equivalent thereof; admixing the O1-180, O1-184 and/or O1-236 polypeptide or functional equivalent thereof with a candidate compound; measuring an effect of said candidate compound on the activity and/or expression of O1-180, O1-184 and/or O1-236, and administering to the subject an effective amount of the modulator to decrease conception and/or increase conception. Thus, the modulator can be a contraceptive or a fertility agent.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1. Multi-tissue Northern blot analysis of ovary-specific genes. Northern blot analysis was performed on total RNA using O1-180, O1-184, and O1-236 probes. These gene products demonstrate an ovary-specific pattern (OV, ovary; WT, wild-type; −/−, Gdf9 knockout) as shown. The migration positions of 18S and 28S ribosomal RNA are indicated. All lanes had approximately equal loading as demonstrated using an 18S rRNA cDNA probe. (Br, brain; Lu, lung; He, heart; St, stomach; Sp, spleen; Li, liver; Si, small intestine; Ki, kidney; Te, testes, Ut, uterus).

FIGS. 2A-2F. In situ hybridization analysis of ovary-specific genes in mouse ovaries. In situ hybridization was performed using anti-sense probes to O1-180 (FIGS. 2A-2B), O1-184 (FIGS. 2C-2D) and O1-236 (FIGS. 2E-2F). FIGS. 2A, 2C, and 2E are brightfield analysis of the ovaries. FIGS. 2B, 2D, and 2F are darkfield analysis of the same ovary sections. All genes demonstrate specific expression in the oocyte beginning at the one layer primary follicle stage (small arrows) and continuing through the antral follicle stage (large arrows).

FIGS. 3A and 3B. In situ hybridization analysis of O1-236 in mouse ovaries. In situ hybridization was performed using probe O1-236 (partial Npm2 cDNA fragment). Brightfield analysis (FIG. 3A) and darkfield analysis (FIG. 3B) of the O1-236 mRNA in the same adult ovary sections. The probe demonstrates specific expression in all growing oocytes. Oocyte-specific expression is first seen in the early one layer primary follicle (type 3a), with higher expression in the one layer type 3b follicle and all subsequent stages including antral (an) follicles.

FIG. 4. Amino acid sequence conservation among Xenopus laevis (SEQ.ID.NO.15), mouse (SEQ.ID.NO.6), rat (SEQ.ID.NO.42) and human (SEQ.ID.NO.9) NPM2 proteins. Using the NCBI blast search tools and Megalign software, comparison of mouse (m), human (h), (r) rat and Xenopus laevis NPM2 amino acid sequences reveals high identity. Spaces between the amino acids indicate gaps to aid in the alignment. Inter-species amino acid identity is highlighted in black. The conserved bipartite nuclear localization sequence is indicated by asterisks (*); a line is drawn over the acidic histone binding region.

FIG. 5. Chromosomal localization of the mouse Npm2 gene. (Top) Map figure from the T31 radiation hybrid database at The Jackson Laboratory showing Chromosome 14 data. The map is depicted with the centromere toward the top. Distances between adjacent loci in centiRay3000 are shown to the left of the chromosome bar. The positions of some of the chromosome 14 MIT markers are shown on the right. The mouse Npm2 gene is positioned between D14Mit203 and D14Mit32. Missing typings were inferred from surrounding data where assignment was unambiguous. (Bottom) Haplotype figure from the T31 radiation hybrid database at The Jackson Laboratory showing part of Chromosome 14 with loci linked to Npm2. Loci are listed in the best fit order with the most proximal at the top. The black boxes represent hybrid cell lines scoring positive for the mouse fragment and the white boxes represent cell lines scoring as negative. The grey box indicates an untyped or ambiguous line. The number of lines with each haplotype is given at the bottom of each column of boxes. Missing typings were inferred from surrounding data where assignment was unambiguous.

FIGS. 6A-6H. Analysis of Npm2 mRNA and NPM2 protein in mouse ovaries and early embryos. In situ hybridization was performed using probe O1-236 (partial Npm2 cDNA fragment). Brightfield analysis (FIG. 6A) and darkfield analysis (FIG. 6B) of the O1-236 mRNA in the same adult ovary sections. Arrows and arrowheads denote expression of the Npm2 mRNA in oocytes from follicles at various stages of follicular development. (FIG. 6C) Immunohistochemistry of ovaries from a 5-week old mouse stained for NPM2 in the nuclei of oocytes from type 3 through to antral follicles. (FIG. 6D) In preovulatory GVB oocytes induced by luteinizing hormone (hCG), NPM2 is evenly stained in the cytoplasm (arrow). An LH (hCG) unresponsive preantral follicle (upper right) continues to demonstrate an oocyte with NPM2 protein localized to the nucleus. (FIG. 6E) After fertilization, NPM2 begins to localize in the pronuclei; the formation of one pronucleus (arrow), is in the process of forming and some of NPM2 staining continues to be present in the cytoplasm of this early one cell embryo. (FIG. 6F) The pronuclei stain strongly in an advanced one cell embryo where very little NPM2 remains in the cytoplasm. NPM2 antibodies also specifically stain the nuclei of two cell (FIG. 6G) and eight cell (FIG. 6H) embryos.

FIGS. 7A-7C. Gene targeting construct for a knockout of Npm2 and genotype analysis of offspring from heterozygote intercrosses. FIG. 7A shows the targeting strategy used to delete exon 2, exon 3, and the junction region of exon 4. PGK-hprt and MC1-tk expression cassettes. Recombination was detected by Southern blot analysis using 5′ and 3′ probes. (B, BamH1; Bg, Bgl II; P, Pst I). FIG. 7B shows a Southern blot analysis of genomic DNA isolated from intercrosses of Npm2^{+/-} mice. The 3′ probe identifies the wild-type 7.5-kb band and the mutant 10.3-kb band when DNA was digested with Bgl II. FIG. 7C shows that when DNA was digested with Pst 1, the exon 2 probe only detected the wild-type 4.5-kb fragment.

FIGS. 8A-8F. Histological analysis of ovaries from wild-type, Npm2^{+/-} and Npm2^{-/-} mice. (FIG. 8A-8D) Immunohistochemistry of ovaries from 6-week old mice stained for Npm2 in the nuclei of oocytes (FIG. 8A and FIG. 8C for Npm2^{+/-} ovaries; FIG. 8B and FIG. 8D for Npm2^{-/-} ovaries). (FIGS. 8E-8F) PAS (Periodic acid Schiff)/hematoxylin staining of ovaries from 12 week old mice wild-type (FIG. 8E) and Npm2^{-/-} (FIG. 8F) ovaries. Arrows show large antral follicles.

FIGS. 9A-9F. In vitro culture of eggs (metaphase II) and fluorescent-labeling of DNA from fertilized eggs from Npm2^{-/-} and control mice. Eggs were isolated from the oviducts of immature mice after superovulation and cultured in vitro. Pictures were taken under a microscope at 45 (FIGS. 9A-9B), 55 (FIGS. 9C-9D) and 96 (FIGS. 9E-9F) hours of culture. Most fertilized eggs from wild-type mice form 2-cell and 4-cell embryos by 45 and 55 hours post-hCG (white arrows), while few Npm2 Npm2^{-/-} eggs cleave to form multicellular embryos, and even fewer form blastocysts compared to wild-type controls.

FIGS. 10 and 10B. The percent cleavage of in vivo fertilized embryos to various stages is shown after oviduct collection (FIG. 10A) and subsequent 24 hour culture (FIG. 10B). Times are given as hours post-hCG.

FIGS. 11A-11D. Wild-type (FIG. 11A) and Npm2^{-/-} (FIG. 10B) fertilized oocytes are TUNEL negative, with the exception of their TUNEL positive polar bodies. (FIGS. 11C and 11D) Later, DNA within fragmenting Npm2 null embryos stain TUNEL positive.

FIG. 12. Transcription-requiring complex (TRC) proteins were extracted from wild-type (WT) and null (−/−) 2-cell embryos after culture in ³⁵S-labeled methionine. As a negative control, actinomycin D (ActD) inhibited transcription and TRC production.

FIGS. 13A-13Z. WT and mutant oocytes and embryos were analyzed. Immunofluorescence analysis of wild-type or Npm2 null oocytes (FIGS. 13A-13J), 1-cell embryos (FIGS. 13K-13V), or 8-cell embryos (FIGS. 13W-13Z) was performed using the indicated antibodies. DNA was counterstained with DAPI (FIGS. 13A-13L, 130-13P, and 13S-13Z) or To-pro-3 (FIGS. 13Q-13R).

FIG. 14. Analysis of ribosomal RNAs is shown in oocytes and 1-cell embryos. An RNAse protection assay was performed to quantify 18S and 28S rRNAs in wild-type (WT) and Npm2 null GV stage oocytes, metaphase II oocytes, and 1-cell embryos. Small quantities of untreated full-length probe served as indicators that the digestion went to completion (Lanes 1 and 8). Phosphorimager analysis to quantify WT and Npm2 null rRNA signals (i.e., comparing Lane 2 and 5; 3 and 6; 4 and 7; 9 and 12; 10 and 13; and 11 and 14) result in ratios ranging from 0.69 to 1.40.

FIG. 15. Absolute levels of protein synthesis in oocytes and 1-cell embryos are shown. In all cases, the addition of 3.0 mg/mL unlabeled methionine competed effectively with the incorporation of the ³⁵S-labeled methionine.

FIGS. 16A-16H. In situ hybridization was used to detect Npm1 and Npm3 mRNAs in ovaries of wild-type mice. Npm1 mRNA was highly expressed in oocytes of small follicles (FIGS. 16A-16B), secondary follicles (FIGS. 16C-16D) and large antral follicles (FIGS. 16E-16F) (arrows). Sections are shown in brightfield (FIGS. 16A, 16C, and 16E) and darkfield (FIGS. 16B, 16D, and 16F) to demonstrate the histology and highlight the hybridization signal, respectively. Npm3 mRNA was detected in all stages of oocytes in the adult ovary (FIGS. 16G-16H).

FIGS. 17A and 17B. Expression analysis of Zygote arrest 1 in mouse and human tissues. FIG. 17A shows a Northern blot analysis with the Zar1 cDNA fragment in total RNA derived from wildtype tissues and Gdf9^{-/-} ovaries. FIG. 17B shows RT-PCR analysis of human ZAR1. (Br, brain; Lu, lung; He, heart; St, stomach; Sp, spleen; Li, liver; SI, small intestine; Ki, kidney; Te, testes; Ut, uterus; Co, colon; Pr, prostate; Pl, placenta; Pa, pancreas; Mu, muscle).

FIG. 18. Comparison of the mouse and human ZAR1 amino acid sequences.

FIGS. 19A and 19B. Comparison of the Zar1 gene and the Zar1-ps1 pseudogene. Sequences of exons, exon-intron boundaries and the size of each intron are shown. Different nucleotides between the two genes and consensus polyadenylation sequence are underlined. The translation start codon and stop codon are shown in bold. Upper case: exon sequences; lower case: intron sequences.

FIGS. 20A and 20B. Maps of mouse chromosome 5, showing the position in centiMorgan (cM) of the marker best linked to the Zar1 gene (FIG. 20A) and its related pseudogene (FIG. 20B).

FIG. 21. Western blot analysis of recombinant ZAR1.

FIGS. 22A-22F. Expression of Zar1 in PMSG-treated wild-type (FIGS. 22A and 22B) and Gdf9^{-/-} (FIGS. 22C-22F) ovaries was analyzed by in situ hybridization with a specific antisense probe. Both brightfield (FIGS. 22A, 22C and 22E) and corresponding darkfield (FIGS. 22B, 22D and 22F) images of the same ovary sections are presented. Areas of sections of FIGS. 22C and 22D are shown at higher magnification (FIGS. 22E and 22F). The expression of the Zar1 gene was detected at early primary follicle (type 3a) through to antral follicle (type 8) stage, but not in primordial follicles (type 2), in wild-type or Gdf9^{-/-} ovaries. In Gdf9^{-/-} ovaries, the follicle numbers increase per unit volume due to follicle arrest at the primary stage, and hence more Zar1 positive signals were detected in each section.

FIGS. 23A-23D. Mouse Zar1 gene structure and targeting strategy. FIG. 23A shows a targeting vector, which was constructed by replacing Exon 1 (which contains the ATG start codon) and part of intron 1 with a PGK-Hprt expression cassette. Targeted ES cell clones containing a wild-type (WT), a pseudogene allele (Zar1-ps1), and a mutant (MUT) allele were confirmed by Southern blot analysis and injected into blastocysts to produce chimeric male mice, which were bred to produce F1 Zar1^{+/-} offspring. Southern blot analysis (FIG. 23B) of genomic DNA is derived from offspring of one litter from a heterozygous mating. FIG. 23C shows Northern blot analysis of ovarian mRNA from wild-type, Zar1^{+/-}, and Zar1^{-/-} females using the full-length Zar1 cDNA. On longer exposure, a smaller transcript of unknown relevance was observed in Zar1^{-/-} ovaries, and the expression level of Zar1 in wild-type mice is approximately twice the levels of the Zar1^{+/-}. Gapdh was used as a control for equal loading on the Northern blot (FIG. 23D).

FIGS. 24A-24J. Mouse ZAR1 protein expression. An anti-mouse ZAR1 polyclonal antibody was used for immunohistochemistry (FIGS. 24A-24D) and immunofluorescence analysis (FIGS. 24E-24J) to detect ZAR1 expression. Similar to the Zar1 mRNA, ZAR1 protein expression begins in oocytes of primary follicles and continues through all follicle stages in wild-type ovaries (FIGS. 24A, 24B). ZAR1 is also detected in Gdf9^{-/-} ovaries (FIG. 24C), whereas no protein was detected in Zar1^{-/-} ovaries (FIG. 24D). ZAR1 protein was detected predominantly in the cytoplasm of fully-grown, prophase I-arrested oocytes from Zar1^{+/-} (FIG. 24E) but not Zar1^{-/-} mice (FIG. 24F). ZAR1 is expressed in wild-type oocytes, during the progression from MI (FIG. 24G) to MII (FIG. 24H), and persists in zygotes at the 1-cell stage, collected 6 h post-fertilization (FIG. 241). However, ZAR1 expression is dramatically reduced in 2-cell stage embryos (FIG. 24J), with bright staining evident only in polar body remnants.

FIGS. 25A-25D. Development of embryos derived from Zar1^{+/-} and Zar1^{-/-} mice. Adult Zar1^{+/-} (FIG. 25A) and Zar1^{-/-} (FIG. 25B) females were mated with stud males. Whereas all zygotes from Zar1^{+/-} female mice progressed to the blastocyst stage (FIG. 25A), most zygotes from Zar1^{-/-} mice remained at the 1-cell stage, and many degenerating embryos were detected (FIG. 25B). At 24 h post-fertilization, the arrested zygotes from Zar1^{-/-} females were labeled with anti-β tubulin and propidium iodide to assess microtubule and chromatin configurations, respectively (FIG. 25C). Decondensed chromatin was evident in both the maternal and paternal pronucleus. Additionally, the microtubules show an interphase configuration, with no assembled spindle apparatus. In a second experiment, the fertilized zygotes were placed in medium with BrdU at 8 h post-fertilization and cultured overnight (FIG. 25D). Immunofluoresence analysis shows BrdU incorporation in both pronuclei of an arrested zygote from a Zar1^{-/-} female indicative of entry into S-phase.

FIGS. 26A-26B. Cell-free transcription/translation of Zar1, Polr2c (DNA directed RNA polymerase II polypeptide C), Gnb2 (Guanine nucleotide binding protein, beta 2), Polr2g (DNA directed RNA polymerase II polypeptide G), and Lmo1 (LIM only 1) cDNAs. Autoradiograph of [³⁵S] Met-labeled proteins from cell-free in vitro transcription/translation and co-immunoprecipitation by anti-HA polyclonal antibody (FIG. 26A) or anti-MYC monoclonal antibody (FIG. 26B). The position of molecular mass standards in kDa is shown at the right. The HA-tagged POLR2C, GNB2, POLR2G, and LMO1 bind to the MYC-tagged ZAR1.

FIG. 27. Amino Acid sequence comparison of ZAR1 proteins from homo sapiens, Mus musculus, Xenopus laevis, Danio rerio and Fugu rubripes.

### DETAILED DESCRIPTION OF THE INVENTION

It is readily apparent to one skilled in the art that various embodiments and modifications can be made to the invention disclosed in this Application without departing from the scope and spirit of the invention.

As used herein, the use of the word “a” or “an” when used in conjunction with the term “comprising” in the claims and/or the specification may mean “one,” but it is also consistent with the meaning of “one or more,” “at least one,” and “one or more than one.”

As used herein, the term “animal” refers to a mammal, such as human, non-human primates, horse, cow, elephant, cat, dog, rat or mouse. In specific embodiments, the animal is a human.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. Thus, one of skill in the art understands that the term “antibody” refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. (See, e.g., Antibodies: A Laboratory Manual, Cold Spring Harbor Laboratory, 1988).

As used herein, the term “binding protein” refers to proteins that demonstrate binding affinity for a specific ligand. Binding proteins may be produced from separate and distinct genes. For a given ligand, the binding proteins that are produced from specific genes are distinct from the ligand binding domain of the receptor or its soluble receptor.

As used herein, the term “binding partner” or “interacting proteins” refer to a molecule capable of binding another molecule with specificity, as for example, an antigen and an antigen-specific antibody or an enzyme and its inhibitor. Binding partners may include, for example, biotin and avidin or streptavidin, IgG and protein A, receptor-ligand couples, protein-protein interaction, and complementary polynucleotide strands. The term “binding partner” may also refer to polypeptides, lipids, small molecules, or nucleic acids that bind to O1-180, O1-236 and/or O1-184 in cells. A change in the interaction between a protein and a binding partner can manifest itself as an increased or decreased probability that the interaction forms, or an increased or decreased concentration of O1-180, O1-236 and/or O1-184 in cells-binding partner complex.

As used herein, the term “O1-180 binding fragment”, “O1-184 binding fragment” and/or “O1-236 binding fragment” refers to the nucleic acid fragment and/or amino acid fragment of O1-180, O1-184 and/or O1-236 respectively that is capable of binding to the binding partner or interacting protein, for example polypeptides, lipids, small molecules, or nucleic acids.

As used herein, the terms “cell,” “cell line,” and “cell culture” may be used interchangeably. All of these terms also include their progeny, which are any and all subsequent generations. It is understood that all progeny may not be identical due to deliberate or inadvertent mutations. In the context of expressing a heterologous nucleic acid sequence, “host cell” refers to a prokaryotic or eukaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal. Host cell can be used as a recipient for vectors and may include any transformable organisms that are capable of replicating a vector and/or expressing a heterologous nucleic acid encoded by a vector.

As used herein, the term “conception” refers to the union of the male sperm and the ovum of the female; fertilization.

As used herein, the term “contraception” refers to the prevention or blocking of conception. A contraceptive device, thus, refers to any process, device, or method that prevents conception. Well known categories of contraceptives include, steroids, chemical barrier, physical barrier; combinations of chemical and physical barriers; use of immunocontraceptive methods by giving either antibodies to the reproductive antigen of interest or by developing a natural immune response to the administered reproductive antigen; abstinence and permanent surgical procedures. Contraceptives can be administered to either males or females.

As used herein, the term “complementary” is used to describe the relationship between nucleotide bases that are capable to hybridizing to one another. For example, with respect to DNA, adenosine is complementary to thymine and cytosine is complementary to guanine.

As used herein, the term “DNA” is defined as deoxyribonucleic acid.

As used herein, “cDNA” refers to DNA that is complementary to and derived from an mRNA template. The cDNA can be single-stranded or converted to double-stranded form using, for example, the Klenow fragment of DNA polymerase I.

As used herein, the term “DNA segment” refers to a DNA molecule that has been isolated free of total genomic DNA of a particular species. Included within the term “DNA segment” are DNA segments and smaller fragments of such segments, and also recombinant vectors, including, for example, plasmids, cosmids, phage, viruses, and the like.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. In the present invention, the term “therapeutic construct” may also be used to refer to the expression construct or transgene. One skilled in the art realizes that the present invention utilizes the expression construct or transgene as a therapy to treat infertility. Yet further, the present invention utilizes the expression construct or transgene as a “prophylactic construct” for contraception. Thus, the “prophylactic construct” is a contraceptive.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described infra.

As used herein, the term “gene” is used for simplicity to refer to a functional protein, polypeptide or peptide encoding unit. This functional term includes both genomic sequences, cDNA sequences and engineered segments that express, or may be adapted to express, proteins, polypeptides, domains, peptides, fusion proteins and mutant. Thus, one of skill in the art is aware that the term “native gene” or “endogenous gene” refers to a gene as found in nature with its own regulatory sequences and the term “chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences that are derived from the same source, but arranged in a manner different than that found in nature.

As used herein, the term “gonadal” or “gonadal tissue” or “gonads” refers to tissue that is related to the male and female sex organs. Gonadal tissue is not limited to the ovaries and/or testes; it may also include the embryonic tissue that develops into the ovaries and/or testes.

As used herein, the terms “identity” or “similarity”, as known in the art, are relationships between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Both identity and similarity can be readily calculated by known methods such as those described in: Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991. Methods commonly employed to determine identity or similarity between sequences include, but are not limited to those disclosed in Carillo, H., and Lipman, D., SIAM J Applied Math., 48:1073 (1988). Methods to determine identity and similarity are codified in publicly available computer programs. Preferred computer program methods to determine identity and similarity between two sequences include, but are not limited to, GCG program package, Devereux, J., et al., Nucleic Acids Research, 12(1), 387 (1984)), BLASTP, BLASTN, and FASTA Atschul, S. F. et al., J Molec. Biol., 215, 403 (1990)).

As used herein, the term “homologous” refers to the degree of sequence similarity between two polymers (i.e. polypeptide molecules or nucleic acid molecules). The homology percentage figures referred to herein reflect the maximal homology possible between the two polymers, i.e., the percent homology when the two polymers are so aligned as to have the greatest number of matched (homologous) positions.

As used herein, the term “percent homology” refers to the extent of amino acid sequence identity between polypeptides. The homology between any two polypeptides is a direct function of the total number of matching amino acids at a given position in either sequence, e.g., if half of the total number of amino acids in either of the sequences are the same then the two sequences are said to exhibit 50% homology.

The term “fragment”, “analog”, and “derivative” when referring to the polypeptide of the present invention (e.g., O1-180 (SEQ.ID.NO.2, SEQ.ID.NO.16, SEQ.ID.NO.29, SEQ.ID.NO.32, SEQ.ID.NO.34, SEQ.ID.NO.36 and SEQ.ID.NO.39), O1-184 (SEQ.ID.NO.4), O1-236 (SEQ.ID.NO.6, SEQ.ID.NO.9, and SEQ.ID.NO.42)), refers to a polypeptide which may retain essentially the same biological function or activity as such polypeptide. Thus, an analog includes a precursor protein that can be activated by cleavage of the precursor protein portion to produce an active mature polypeptide. The fragment, analog, or derivative of the polypeptide of the present invention (O1-180 (SEQ.ID.NO.2, SEQ.ID.NO.16, SEQ.ID.NO.29, SEQ.ID.NO.32, SEQ.ID.NO.34, SEQ.ID.NO.36 and SEQ.ID.NO.39), O1-184 (SEQ.ID.NO.4), O1-236 (SEQ.ID.NO.6, SEQ.ID.NO.9, and SEQ.ID.NO.42)), may be one in which one or more of the amino acids are substituted with a conserved or non-conserved amino acid residues and such amino acid residues may or may not be one encoded by the genetic code, or one in which one or more of the amino acid residues includes a substituent group, or one in which the polypeptide is fused with a compound such as polyethylene glycol to increase the half-life of the polypeptide, or one in which additional amino acids are fused to the polypeptide such as a signal peptide or a sequence such as polyhistidine tag which is employed for the purification of the polypeptide or the precursor protein. Such fragments, analogs, or derivatives are deemed to be within the scope of the present invention.

The term “functional equivalent” as used herein is defined as a polynucleotide that has been engineered to contain distinct sequences while at the same time retaining the capacity to perform the biologic function of interest of the wild-type or reference protein. Thus, as used herein, the term functional equivalent includes truncations, deletions, insertions or substitutions of O1-180 (SEQ.ID.NO.2, SEQ.ID.NO.16, SEQ.ID.NO.29, SEQ.ID.NO.32, SEQ.ID.NO.34, SEQ.ID.NO.36 and SEQ.ID.NO.39), O1-184 (SEQ.ID.NO.4), O1-236 (SEQ.ID.NO.6, SEQ.ID.NO.9, and SEQ.ID.NO.42)) which retains their function to play a role in in fertility and embryonic development. This also can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a protein. In another example, a polynucleotide may be (and encode) a functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention.

The term “hyperproliferative disease” is defined as a disease that results from a hyperproliferation of cells. Hyperproliferative disease is further defined as cancer. The hyperproliferation of cells results in unregulated growth, lack of differentiation, local tissue invasion, and metastasis. Exemplary hyperproliferative diseases include, but are not limited to cancer or autoimmune diseases. Other hyperproliferative diseases can include vascular occlusion, restenosis, atherosclerosis, or inflammatory bowel disease.

As used herein, the term “fertility” refers to the quality of being productive or able to conceive. Fertility relates to both male and female animals.

As used herein, the term “infertility” refers to the inability or diminished ability to conceive or produce offspring. Infertility can be present in either male or female. In the present invention, administration of a composition to enhance infertility or decrease fertility is reversible. Examples of infertility include, without limitation, azoospermia; genetic disorders associated with defective spermatogenesis (e.g., Klinefelter's syndrome and gonadal dysgenesis); oligospermia, varicocele, and other sperm disorders relating to low sperm counts, sperm motility, and sperm morphology; and ovulatory dysfunction (e.g., polycystic ovary syndrome (PCOS) or chronic anovulation).

As used herein, the terms “O1-180”, “Oo1”, “zygote arrest 1 (Zar1)”, “ZAR1” or “ZAR1” are interchangeable. Zar1 and ZAR1 denote the mouse and human DNA sequence, respectively. ZAR1 denotes the mouse and the human amino acid sequences.

As used herein, the terms “O1-236”, “nucleoplasmin 2 (Npm2)”, “NPM2” or “NPM2” are interchangeable. Npm2 and NPM2 denote the mouse and human DNA sequence, respectively. NPM2 denotes the mouse and the human amino acid sequences.

As used herein, the term “modulate” refers to the suppression, enhancement, or induction of a function. For example, “modulation” or “regulation” of gene expression refers to a change in the activity of a gene. Modulation of expression can include, but is not limited to, gene activation and gene repression. “Modulate” or “regulate” also refers to methods, conditions, or agents which increase or decrease the biological activity of a protein, enzyme, inhibitor, signal transducer, receptor, transcription activator, co-factor, and the like. This change in activity can be an increase or decrease of mRNA translation, DNA transcription, and/or mRNA or protein degradation, which may in turn correspond to an increase or decrease in biological activity. Such enhancement or inhibition may be contingent upon occurrence of a specific event, such as activation of a signal transduction pathway and/or may be manifest only in particular cell types.

As used herein, the term “modulated activity” refers to any activity, condition, disease or phenotype that is modulated by a biologically active form of a protein. Modulation may be affected by affecting the concentration of biologically active protein, e.g., by regulating expression or degradation, or by direct agonistic or antagonistic effect as, for example, through inhibition, activation, binding, or release of substrate, modification either chemically or structurally, or by direct or indirect interaction which may involve additional factors.

As used herein, the term “modulator” refers to any composition and/or compound that alters the expression of a specific activity, such as O1-236 activity or expression, O-180 activity or expression, and/or O1-184 activity or expression. The modulator is intended to comprise any composition or compound, e.g., antibody, small molecule, peptide, oligopeptide, polypeptide, or protein.

The term “small molecule” refers to a synthetic or naturally occurring chemical compound, for instance a peptide or oligonucleotide that may optionally be derivatized, natural product or any other low molecular weight (typically less than about 5 kDalton) organic, bioinorganic or inorganic compound, of either natural or synthetic origin. Such small molecules may be a therapeutically deliverable substance or may be further derivatized to facilitate delivery.

The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (i.e., that the coding sequence is under the transcriptional control of the promoter). Coding sequences can be operatively linked to regulatory sequences in sense or antisense orientation. As used herein, the term “peptide binding pair” refers to any pair of peptides having a known binding affinity for which the DNA sequence is known or can be deduced. The peptides of the peptide binding pair must exhibit preferential binding for each other over any other components of the modified cell.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic and/or prophylactic compositions is contemplated. Supplementary active ingredients also can be incorporated into the compositions.

As used herein, the terms “polynucleotide”, “nucleotide sequence”, “nucleic acid”, “nucleic acid molecule”, “nucleic acid sequence”, “oligonucleotide”, refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil, or containing carbohydrate, or lipids.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

As used herein, the term “purified protein or peptide”, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

As used herein, the term “RNA” is defined as ribonucleic acid.

As used herein, “messenger RNA (mRNA)” refers to the RNA that is without introns and can be translated into polypeptides by the cell.

As used herein, the term “RNA interference” or “RNAi” is an RNA molecule that is used to inhibit a particular gene of interest.

As used herein, the term “regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

As used herein, the term “sense” refers to sequences of nucleic acids that are in the same orientation as the coding mRNA nucleic acid sequence. A DNA sequence linked to a promoter in a “sense orientation” is linked such that an RNA molecule which contains sequences identical to an mRNA is transcribed. The produced RNA molecule, however, need not be transcribed into a functional protein.

As used herein, the term an “anti-sense” copy of a particular polynucleotide refers to a complementary sequence that is capable of hydrogen bonding to the polynucleotide and can therefor be capable of modulating expression of the polynucleotide. These are DNA, RNA or analogs thereof, including analogs having altered backbones, as described above. The polynucleotide to which the anti-sense copy binds may be in single-stranded form or in double-stranded form. A DNA sequence linked to a promoter in an “anti-sense orientation” may be linked to the promoter such that an RNA molecule complementary to the coding mRNA of the target gene is produced.

As used herein, the terms “sense” strand and an “anti-sense” strand when used in the same context refer to single-stranded polynucleotides that are complementary to each other. They may be opposing strands of a double-stranded polynucleotide, or one strand may be predicted from the other according to generally accepted base-pairing rules. Unless otherwise specified or implied, the assignment of one or the other strand as “sense” or “antisense” is arbitrary.

The term “effective amount” or “therapeutically effective amount” as used herein refers to an amount that results in an improvement or remediation of the symptoms of the disease or condition.

The term “treating” and “treatment” as used herein refers to administering to a subject a therapeutically effective amount of the pharmaceutical composition and/or modulator so that the subject has an improvement in the disease and/or condition. The improvement is any improvement or remediation of the symptoms. The improvement is an observable or measurable improvement. Thus, one of skill in the art realizes that a treatment may improve the disease and/or condition, but may not be a complete cure for the disease and/or condition.

As used herein, the term “under transcriptional control” or “operatively linked” is defined as the promoter that is in the correct location and orientation in relation to the nucleic acid to control RNA polymerase initiation and expression of the gene.

The present invention provides three novel proteins, O1-180 (SEQ.ID.NO.2, SEQ.ID.NO.16, SEQ.ID.NO.29, SEQ.ID.NO.32, SEQ.ID.NO.34, SEQ.ID.NO.36 and SEQ.ID.NO.39), O1-184 (SEQ.ID.NO.4), O1-236 (SEQ.ID.NO.6, SEQ.ID.NO.9, and SEQ.ID.NO.42), the polynucleotide sequences that encode them, and fragments and derivatives thereof. Expression of O1-180, O1-184, O1-236 is highly tissue-specific, being expressed in cells primarily of ovarian tissue. In one embodiment, the invention provides a method for detection of a cell proliferative or degenerative disorder of the ovary, which is associated with expression of O1-180, O1-184 or O1-236. In another embodiment, the invention provides a method for treating a cell proliferative or degenerative disorder associated with abnormal expression of O1-O1-180, O1-184, O1-236 by using an agent which suppresses or enhances their respective activities.

Based on the known activities of many other ovary specific proteins, it can be expected that O1-180, O1-184 and O1-236, as well as fragments and derivatives thereof, will also possess biological activities that will make them useful as diagnostic and therapeutic reagents.

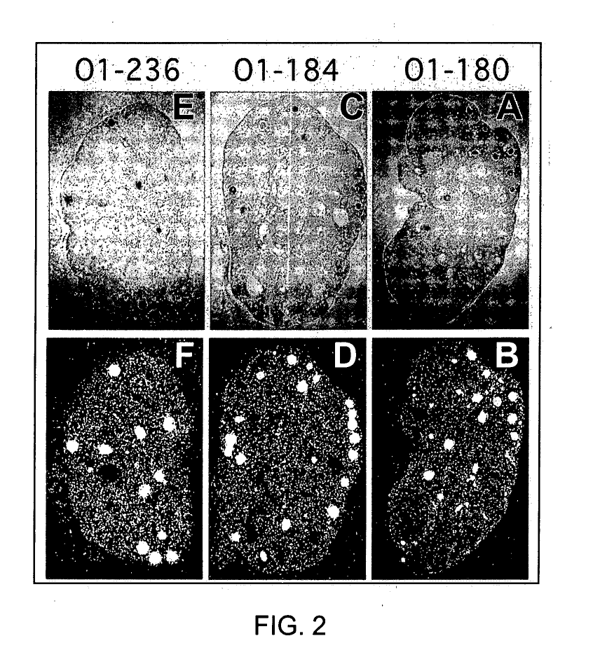

For example, GDF-9 is an oocyte-expressed gene product which has a similar pattern of expression as O1-180, O1-184, and O1-236. It has been shown that mice lacking GDF-9 are infertile at a very early stage of follicular development, at the one-layer primary follicle stage. These studies demonstrate that agents which block GDF-9 function would be useful as contraceptive agents in human females. Since O1-180, O1-184, and O1-236 have an expression pattern in the oocyte (FIG. 2) which is nearly identical to GDF-9, this suggests that mice and humans or any other mammal lacking any of all of these gene products may also be infertile. Thus, blocking the function of any or all of these gene products may result in a contraceptive action.

Another regulatory protein that has been found to have ovary-specific expression is inhibin, a specific and potent polypeptide inhibitor of the pituitary secretion of FSH. Inhibin has been isolated from ovarian follicular fluid. Because of its suppression of FSH, inhibin has been advanced as a potential contraceptive in both males and females. O1-180, O1-184 and O1-236 may possess similar biological activity since they are also ovarian specific peptides. Inhibin has also been shown to be useful as a marker for certain ovarian tumors (Lappohn et al., 1989). O1-180, O1-184, O1-236 may also be useful as markers for identifying primary and metastatic neoplasms of ovarian origin. Likewise, mice which lack inhibin develop granulosa cell tumors (Matzuk et al., 1992). Similarly, O1-180, O1-184 and O1-236 may be useful as indicators of developmental or reproductive anomalies in prenatal screening procedures.

Mullerian inhibiting substance (MIS or anti-Mullerian hormone) peptide, which is produced by the testis and is responsible for the regression of the Mullerian ducts in the male embryo, has been shown to inhibit the growth of human ovarian cancer in nude mice (Donahoe et al., 1981). O1-180, O1-184 and O1-236 may function similarly and may, therefore, be targets for anti-cancer agents, such as for the treatment of ovarian cancer.

O1-180, O1-184 and O1-236, and agonists and antagonists thereof can be used to identify agents which inhibit fertility (e.g., act as a contraceptive) in a mammal (e.g., human). Additionally, O1-180, O1-184 and O1-236 and agonists and antagonists thereof can be used to identify agents which enhance fertility (e.g., increase the success of in vivo or in vitro fertilization) in a mammal. Likewise, assays of these or related oocyte-expressed gene products can be used in diagnostic assays for detecting forms of infertility (e.g., in an assay to analyze activity of these gene products) or other diseases (e.g., germ cell tumors, polycystic ovary syndrome).

### I. PROTEINS

In an effort to identify other novel ovarian-expressed genes that may play key functions in ovarian physiology, fertilization and early cleavage events, the inventors used a subtractive hybridization approach. Several novel oocyte-expressed genes have been identified by the inventors which are important in regulating oogenesis, folliculogenesis, fertilization, and/or early embryogenesis. One of these oocyte-specific gene products, nucleoplasmin 2 (O1-236 or NPM2), is the mammalian ortholog of Xenopus laevis nucleoplasmin (xNPM2) (Burglin et al., 1987; Dingwall et al., 1987). The 207 amino acid open reading frame of NMP2 demonstrated high homology to the family of proteins called nucleoplasmins or nucleophosmins (nomenclature designation=species). NPM2 human gene, Npm2 mouse gene, and Xnpm2 Xenopus gene; NPM2=protein in all species). Human nucleoplasmin gene (NPM1 also called NO38; accession # M23613) maps to human chromosome 5q35, encodes a 294 amino acid protein, and has orthologs in mouse (Npm1, also called B23, accession # Q61937) and Xenopus laevis (Xnpm1 or N038 accession # X05496). Mouse nucleoplasmin/nucleophosmin homolog Npm3, which has been mapped to mouse chromosome 19, encodes a protein of 175 amino acids [accession # U64450, (MacArthur and Shackleford, 1997a)], and there is an apparent human NPM3 homolog gene (accession # AF081280). In contrast to Npm2, the genes Npm1 and Npm3 are ubiquitously expressed, and the structure of the mouse Npm2 gene is considerably divergent compared to the mouse Npm3 gene (MacArthur and Shackleford, 1997a).

In the present invention, O1-180 (SEQ.ID.NO.2, SEQ.ID.NO.16, SEQ.ID.NO.29, SEQ.ID.NO.32, SEQ.ID.NO.34, SEQ.ID.NO.36, and SEQ.ID.NO.39), O1-184 (SEQ.ID.NO.4) and O1-236 (SEQ.ID.NO6, SEQ.ID.NO.9, and SEQ.ID.NO.42) identified these proteins using subtractive hybridization. These identified proteins or agents which act on these pathways may also function as growth stimulatory factors and, therefore, be useful for the survival of various cell populations in vitro. In particular, if O1-180, O1-184 and/or O1-236 play a role in oocyte maturation, they may be useful targets for in vitro fertilization procedures, e.g., in enhancing the success rate.

In this patent, the terms “O1-180 gene product” “O1-184 gene product” and “O1-236 gene product” refer to proteins and polypeptides having amino acid sequences that are substantially identical to the native O1-180, O1-184 and/or O1-236 amino acid sequences (or RNA, if applicable) or that are biologically active, in that they are capable of performing functional activities similar to an endogenous O1-180, O1-184 and/or O1-236 and/or cross-reacting with anti-O1-180, O1-184 and/or O1-236 antibody raised against O1-180, O1-184 and/or O1-236.

The terms “O1-180 gene product” “O1-184 gene product” and “O1-236 gene product” also include analogs of the respective molecules that exhibit at least some biological activity in common with their native counterparts. Such analogs include, but are not limited to, truncated polypeptides and polypeptides having fewer amino acids than the native polypeptide.

In addition to the entire O1-180, O1-184 or O1-236 molecules, the present invention also relates to fragments of the polypeptides that may or may not retain the functions described below. Fragments, including the N-terminus of the molecule, may be generated by genetic engineering of translation stop sites within the coding region. Alternatively, treatment of the O1-180, O1-184 or O1-236 with proteolytic enzymes, known as proteases, can produce a variety of N-terminal, C-terminal and internal fragments. Fragments of proteins are seen to include any peptide that contains 6 contiguous amino acids or more that are identical to 6 contiguous amino acids of sequences of SEQ.ID.NO.2, SEQ.ID.NO.4, SEQ.ID.NO.6, SEQ.ID.NO.9, SEQ.ID.NO.16, SEQ.ID.NO.29, SEQ.ID.NO.32, SEQ.ID.NO.34, SEQ.ID.NO.36, SEQ.ID.NO.39, and SEQ.ID.NO.42. Fragments that contain 7, 8, 9, 10, 11, 12, 13, 14 15, 16, 17, 18, 19, 20, 21, 22, 23, 24, 25, 30, 35, 40, 45, 50, 55, 60, 65, 75, 80, 85, 90, 95, 100, 200 or more contiguous amino acids or more that are identical to a corresponding number of amino acids of any of the sequences of SEQ.ID.NO.2, SEQ.ID.NO.4, SEQ.ID.NO.6, SEQ.ID.NO.9, SEQ.ID.NO.16, SEQ.ID.NO.29, SEQ.ID.NO.32, SEQ.ID.NO.34, SEQ.ID.NO.36, SEQ.ID.NO.39, and SEQ.ID.NO.42 are also contemplated. Fragments may be used to generate antibodies. Particularly useful fragments will be those that make up domains of O1-180, O1-184 or O1-236. Domains are defined as portions of the proteins having a discrete tertiary structure and that is maintained in the absence of the remainder of the protein. Such structures can be found by techniques known to those skilled in the art. The protein is partially digested with a protease such as subtilisin, trypsin, chymotrypsin or the like and then subjected to polyacrylamide gel electrophoresis to separate the protein fragments. The fragments can then be transferred to a PVDF membrane and subjected to micro sequencing to determine the amino acid sequence of the N-terminal of the fragments.

The term substantially pure as used herein refers to O1-180, O1-184 and O1-236 which are substantially free of other proteins, lipids, carbohydrates or other materials with which they are naturally associated. One skilled in the art can purify O1-180, O1-184 and O1-236 using standard techniques for protein purification. The substantially pure polypeptide will yield a single major band on a non-reducing polyacrylamide gel. The purity of the O1-180, O1-184 and O1-236 polypeptides can also be determined by amino-terminal amino acid sequence analysis. O1-180, O1-184 and O1-236 polypeptides include functional fragments of the polypeptides, as long as their activities remain. Smaller peptides containing the biological activities of O1-180, O1-184 and O1-236 may also be used in the present invention.

A. Variants

Amino acid sequence variants of the O1-180, O1-236 and/or O1-184 polypeptides can be substitutional, insertional or deletion variants. Deletion variants lack one or more residues of the native protein which are not essential for function or immunogenic activity. Insertional mutants typically involve the addition of material at a non-terminal point in the polypeptide. This may include the insertion of an immunoreactive epitope or simply a single residue. Terminal additions, called fusion proteins, are discussed below.

The polypeptides of the invention include the disclosed sequences and conservative variations thereof. The term conservative variation as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic acid for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

B. Domain Switching

An interesting series of mutants can be created by substituting homologous regions of various proteins. This is known, in certain contexts, as “domain switching.”

Domain switching involves the generation of chimeric molecules using different but, in this case, related polypeptides. By comparing various O1-180, O1-236 and/or O1-184 proteins or polypeptides, one can make predictions as to the functionally significant regions of these molecules. It is possible, then, to switch related domains of these molecules in an effort to determine the criticality of these regions to O1-180, O1-236 and/or O1-184 function. These molecules may have additional value in that these “chimeras” can be distinguished from natural molecules, while possibly providing the same function.

C. Fusion Proteins

A specialized kind of insertional variant is the fusion protein. This molecule generally has all or a substantial portion of the native molecule, linked at the N- or C-terminus, to all or a portion of a second polypeptide. For example, a fusion protein of the present invention can includes the addition of a protein transduction domains, for example, but not limited to Antennepedia transduction domain (ANTP), HSV1 (VP22) and HIV-1(Tat). Fusion proteins containing protein transduction domains (PTDs) can traverse biological membranes efficiently, thus delivering the protein of interest (O1-180, O1-236 and/or O1-184 or variant thereof, such as an activator or inhibitor) into the cell. (Tremblay, 2001; Forman et al., 2003).

Yet further, inclusion of a cleavage site at or near the fusion junction will facilitate removal of the extraneous polypeptide after purification. Other useful fusions include linking of functional domains, such as active sites from enzymes, glycosylation domains, other cellular targeting signals or transmembrane regions.

D. Synthetic Peptides

The present invention also describes smaller O1-180, O1-236 and/or O1-184-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

E. Antigen Compositions

The present invention also provides for the use of O1-180, O1-236 and/or O1-184 proteins or polypeptides as antigens for the immunization of animals relating to the production of antibodies. Antibodies, which consist essentially of pooled monoclonal antibodies with different epitopic specificities, as well as distinct monoclonal antibodies, are provided. Monoclonal antibodies are made from antigen containing fragments of the protein by methods well known to those skilled in the art (Kohler et al., Nature, 256:495, 1975). The term antibody as used in this invention is meant to include intact molecules as well as fragments thereof, such as Fab and F(ab′)2, which are capable of binding an epitopic determinant on O1-180, O1-184 or O1-236.

It is envisioned that O1-180, O1-236 and/or O1-184 proteins, polypeptides or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyanin (KLH) or bovine serum albumin (BSA).

### 1. Antibody Production

In certain embodiments, the present invention provides antibodies that bind with high specificity to the O1-180, O1-236 and/or O1-184 polypeptides provided herein. Thus, antibodies that bind to the polypeptide of O1-180 (SEQ.ID.NO.2, SEQ.ID.NO.16, SEQ.ID.NO.29, SEQ.ID.NO.32, SEQ.ID.NO.34, SEQ.ID.NO.36 and SEQ.ID.NO.39), O1-184 (SEQ.ID.NO.4), O1-236 (SEQ.ID.NO.6, SEQ.ID.NO.9, and SEQ.ID.NO.42) are provided. In addition to antibodies generated against the full length proteins, antibodies may also be generated in response to smaller constructs comprising epitopic core regions, including wild-type and mutant epitopes.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred. However, humanized antibodies are also contemplated, as are chimeric antibodies from mouse, rat, or other species, bearing human constant and/or variable region domains, bispecific antibodies, recombinant and engineered antibodies and fragments thereof.

A polyclonal antibody is prepared by immunizing an animal with an immunogenic O1-180, O1-236 and/or O1-184 composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified O1-180, O1-236 and/or O1-184 protein, polypeptide, peptide or domain, be it a wild-type or mutant composition. The immunizing composition is administered in a manner effective to stimulate antibody producing cells.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods is also appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods, which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in E. coli.

### 2. Antibody Conjugates

The present invention further provides antibodies against O1-180, O1-236 and/or O1-184, generally of the monoclonal type, that are linked to one or more other agents to form an antibody conjugate. Any antibody of sufficient selectivity, specificity and affinity may be employed as the basis for an antibody conjugate. Such properties may be evaluated using conventional immunological screening methodology known to those of skill in the art.

Certain examples of antibody conjugates are those conjugates in which the antibody is linked to a detectable label. “Detectable labels” are compounds or elements that can be detected due to their specific functional properties, or chemical characteristics, the use of which allows the antibody to which they are attached to be detected, and further quantified if desired. Another such example is the formation of a conjugate comprising an antibody linked to a cytotoxic or anti-cellular agent, as may be termed “immunotoxins” (described in U.S. Pat. Nos. 5,686,072, 5,578,706, 4,792,447, 5,045,451, 4,664,911 and 5,767,072, each incorporated herein by reference).

Antibody conjugates are thus preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In the case of radioactive isotopes for therapeutic and/or diagnostic application, one might mention ²¹¹astatine, ¹⁴carbon, ⁵¹chromium, ³⁶chlorine, ⁵⁷cobalt, ⁵⁸cobalt, ⁶⁷copper, ¹⁵²Eu, ⁶⁷gallium, ³hydrogen, ¹²³iodine, ¹²⁵iodine, ¹³¹iodine, ¹¹¹indium, ⁵⁹iron, ³²phosphorus, ¹⁸⁶rhenium, ¹⁸⁸rhenium, ⁷⁵selenium, ³⁵sulphur, and ^{99m}technicium. ¹²⁵I is often being preferred for use in certain embodiments, and ^{99m}technicium and ¹¹¹indium are also often preferred due to their low energy and suitability for long range detection.

Minor modifications of the recombinant O1-180, O1-184 and O1-236 primary amino acid sequences may result in proteins which have substantially equivalent activity as compared to the respective O1-180, O1-184 and O1-236 polypeptides described herein. Such modifications may be deliberate, as by site-directed mutagenesis, or may be spontaneous. All of the polypeptides produced by these modifications are included herein as long as the biological activity of O1-180, O1-184 or O1-236 still exists. Further, deletion of one or more amino acids can also result in a modification of the structure of the resultant molecule without significantly altering its biological activity. This can lead to the development of a smaller active molecule which would have broader utility. For example, one could remove amino or carboxy terminal amino acids which may not be required for biological activity of O1-180, O1-184 or O1-236.

For the purpose of this invention, the term derivative shall mean any molecules which are within the skill of the ordinary practitioner to make and use, which are made by modifying the subject compound, and which do not destroy the activity of the derivatized compound. Compounds which meet the foregoing criteria which diminish, but do not destroy, the activity of the derivatized compound are considered to be within the scope of the term derivative. Thus, according to the invention, a derivative of a compound comprising amino acids in a sequence corresponding to the sequence of O1-180, O1-184 or O1-236, need not comprise a sequence of amino acids that corresponds exactly to the sequence of O1-180, O1-184 or O1-236, so long as it retains a measurable amount of the activity of the O1-180, O1-184 or O1-236.

Equally, the same considerations may be employed to create a protein, polypeptide or peptide with countervailing, e.g., antagonistic properties. This is relevant to the present invention in which O1-180, O1-184 or O1-236 mutants or analogues may be generated. For example, a O1-180, O1-184 or O1-236 mutant may be generated and tested for O1-180, O1-184 or O1-236 activity to identify those residues important for O1-180, O1-184 or O1-236 activity. O1-180, O1-184 or O1-236 mutants may also be synthesized to reflect a O1-180, O1-184 or O1-236 mutant that occurs in the human population and that is linked to the development of cancer. Also, O1-180, O1-184 or O1-236 mutants may be used as antagonists to inhibit or enhance fertility. Thus, O1-180, O1-184 or O1-236 mutants may be used as potential contraceptive compositions and/or fertility enhancement compositions.

### II. NUCLEIC ACIDS

The term “O1-180 gene” “O1-180 polynucleotide” or “O1-180 nucleic acid” refers to any DNA sequence that is substantially identical to a DNA sequence encoding an O1-180 gene product as defined above. Similar terms for O1-184 and/or O1-236 are within the scope of the present invention. The term also refers to RNA or antisense sequences compatible with such DNA sequences. An “O1-180, O1-184 or O1-236 gene or O1-180, O1-184 or O1-236 polynucleotide” may also comprise any combination of associated control sequences.

Thus, nucleic acid compositions encoding O1-180, O1-184 and/or O1-236 are herein provided and are also available to a skilled artisan at accessible databases, including the National Center for Biotechnology Information's GenBank database and/or commercially available databases, such as from Celera Genomics, Inc. (Rockville, Md.). Also included are splice variants that encode different forms of the protein, if applicable. The nucleic acid sequences may be naturally occurring or synthetic.

As used herein, the terms “O1-180, O1-184 and/or O1-236 nucleic acid sequence,” “O1-180, O1-184 and/or O1-236 polynucleotide,” and “O1-180, O1-184 and/or O1-236 gene” refer to nucleic acids provided herein, homologs thereof, and sequences having substantial similarity and function, respectively. A skilled artisan recognizes that the sequences are within the scope of the present invention if they encode a product which regulates at least one of the following functions oocyte maturation and furthermore knows how to obtain such sequences, as is standard in the art.

Specific polynucleotides of the present invention include sequences encoding the O1-180 (SEQ.ID.NO.1, SEQ.ID.NO.11, SEQ.ID.NO.13, SEQ.ID.NO.12, SEQ.ID.NO.28 (accession # AY191415), SEQ.ID.NO.30 (accession # AY191416), SEQ.ID.NO.31, SEQ.ID.NO.33, SEQ.ID.NO.35, SEQ.ID.NO.37, SEQ.ID.NO.38, SEQ.ID.NO.40 (accession number AY193889) and SEQ.ID.NO.41 (accession # AY193890)), O1-184 (SEQ.ID.NO.3) or O1-236 (SEQ.ID.NO.5, SEQ.ID.NO.7, SEQ.ID.NO.8, SEQ.ID.NO.10, SEQ.ID.NO.14, and SEQ.ID.NO.43) proteins and fragments and derivatives thereof. These polynucleotides include DNA, cDNA and RNA sequences which encode O1-180, O1-184 or O1-236. It is understood that all polynucleotides encoding all or a portion of O1-180, O1-184 and/or O1-236 are also included herein, as long as they encode a polypeptide with the activity of O1-180 (SEQ.ID.NO.1, SEQ.ID.NO.11, SEQ.ID.NO.13, SEQ.ID.NO.12, SEQ.ID.NO.28, SEQ.ID.NO.30, SEQ.ID.NO.31, SEQ.ID.NO.33, SEQ.ID.NO.35, SEQ.ID.NO.37, SEQ.ID.NO.38, SEQ.ID.NO.40 and SEQ.ID.NO.41), O1-184 (SEQ.ID.NO.3) or O1-236 (SEQ.ID.NO.5, SEQ.ID.NO.7, SEQ.ID.NO.8, SEQ.ID.NO.10, SEQ.ID.NO.14 and SEQ.ID.NO.43). Such polynucleotides include naturally occurring, synthetic, and intentionally manipulated polynucleotides. For example, polynucleotides of O1-180 (SEQ.ID.NO.1, SEQ.ID.NO.11, SEQ.ID.NO.13, SEQ.ID.NO.12, SEQ.ID.NO.28, SEQ.ID.NO.30, SEQ.ID.NO.31, SEQ.ID.NO.33, SEQ.ID.NO.35, SEQ.ID.NO.37, SEQ.ID.NO.38, SEQ.ID.NO.40 and SEQ.ID.NO.41), O1-184 (SEQ.ID.NO.3) or O1-236 (SEQ.ID.NO.5, SEQ.ID.NO.7, SEQ.ID.NO.8, SEQ.ID.NO.10, SEQ.ID.NO.14, SEQ.ID.NO,43) may be subjected to site-directed mutagenesis. The polynucleotide sequences for O1-180, O1-184 and O1-236 also includes antisense sequences. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequences of O1-180, O1-184 and O1-236 polypeptides encoded by the nucleotide sequences are functionally unchanged.

The term “substantially identical” and/or “homologous”, when used to define either a O1-180, O1-184 and/or O1-236 amino acid sequence or O1-180, O1-184 and/or O1-236 polynucleotide sequence, means that a particular subject sequence, for example, a mutant sequence, varies from the sequence of natural O1-180, O1-184 and/or O1-236, respectively, by one or more substitutions, deletions, or additions, the net effect of which is to retain at least some biological activity of the O1-180, O1-184 and/or O1-236 protein, respectively. Alternatively, DNA analog sequences are “substantially identical” and/or “homologous” to specific DNA sequences disclosed herein if: (a) the DNA analog sequence is derived from coding regions of the natural O1-180, O1-184 and/or O1-236 gene, respectively; or (b) the DNA analog sequence is capable of hybridization of DNA sequences of (a) under moderately stringent conditions and which encode biologically active O1-180, O1-184 and/or O1-236, respectively; or (c) DNA sequences which are degenerative as a result of the genetic code to the DNA analog sequences defined in (a) or (b). Substantially identical analog proteins will be greater than about 40%, about 45%, about 50%, about 55%, about 60%, about 65% about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein similar to the corresponding sequence of the native protein. Sequences having lesser degrees of similarity but comparable biological activity are considered to be equivalents. In determining polynucleotide sequences, all subject polynucleotide sequences capable of encoding substantially similar amino acid sequences are considered to be substantially similar to a reference polynucleotide sequence, regardless of differences in codon sequence.

A. Complentary Nucleic Acids

The present invention also encompasses a nucleic acid that is complementary to a O1-180, O1-184 and/or O1-236 nucleic acid. In particular embodiments the invention encompasses a nucleic acid or a nucleic acid segment complementary to the sequence set forth in SEQ ID NO: O1-180 (SEQ.ID.NO.1, SEQ.ID.NO.11, SEQ.ID.NO.13, SEQ.ID.NO.12, SEQ.ID.NO.28, SEQ.ID.NO.30, SEQ.ID.NO.31, SEQ.ID.NO.33, SEQ.ID.NO.35, SEQ.ID.NO.37, SEQ.ID.NO.38, SEQ.ID.NO.40 and SEQ.ID.NO.41), O1-184 (SEQ.ID.NO.3) or O1-236 (SEQ.ID.NO.5, SEQ.ID.NO.7, SEQ.ID.NO.8, SEQ.ID.NO.10, SEQ.ID.NO.14, SEQ.ID.NO,43). A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

B. Hybridization of Nucleic Acids

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s)” or “moderately stringent conditions”.

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium or moderate stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suite a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

DNA sequences of the invention can be obtained by several methods. For example, the DNA can be isolated using hybridization or amplification techniques which are well known in the art. These include, but are not limited to: 1) hybridization of genomic or cDNA libraries with probes to detect homologous nucleotide sequences, 2) antibody screening of expression libraries to detect cloned DNA fragments with shared structural features, or 3) use of oligonucleotides related to these sequences and the technique of the polymerase chain reaction.

Preferably, the O1-180, O1-184 and O1-236 polynucleotides of the invention are derived from a mammalian organism, and most preferably from a mouse, rat, elephant, pig, cow or human. Screening procedures which rely on nucleic acid hybridization make it possible to isolate any gene sequence from any organism, provided the appropriate probe is available. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA done by the hybridization of the target DNA to that single probe in the mixture which is its complete complement (Wallace et al., 1981).

The development of specific DNA sequences encoding O1-180, O1-184 and O1-236 can also be obtained by: 1) isolation of double-stranded DNA sequences from the genomic DNA; 2) chemical manufacture of a DNA sequence to provide the necessary codons for the polypeptides of interest; and 3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA.

Of the three above-noted methods for developing specific DNA sequences for use in recombinant procedures, the isolation of genomic DNA isolates is the least common. This is especially true when it is desirable to obtain the microbial expression of mammalian polypeptides due to the presence of introns.

The synthesis of DNA sequences is frequently the method of choice when the entire sequence of amino acid residues of the desired polypeptide product is known. When the entire sequence of amino acid residues of the desired polypeptides is not known, the direct synthesis of DNA sequences is not possible and the method of choice is the synthesis of cDNA sequences. Among the standard procedures for isolating cDNA sequences of interest is the formation of plasmid- or phage-carrying cDNA libraries, which are derived from reverse transcription of mRNA which is abundant in donor cells that have a high level of genetic expression. When used in combination with polymerase chain reaction technology, even rare expression products can be cloned. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay et al., 1983).

A cDNA expression library, such as lambda gt11, can be screened indirectly for O1-180, O1-184 and/or O1-236 peptides having at least one epitope, using antibodies specific for O1-180, O1-184 and/or O1-236. Such antibodies can be either polyclonally or monoclonally derived and used to detect expression product indicative of the presence of O1-180, O1-184 and/or O1-236 cDNA.

### III. EXPRESSION VECTORS

DNA sequences encoding O1-180, O1-184 or O1-236 can be expressed in vitro by DNA transfer into a suitable host cell. Host cells are cells in which a vector can be propagated and its DNA expressed. The term also includes any progeny of the subject host cell. It is understood that all progeny may not be identical to the parental cell since there may be mutations that occur during replication. However, such progeny are included when the term host cell is used. Methods of stable transfer, meaning that the foreign DNA is continuously maintained in the host, are known in the art.

In the present invention, the O1-180, O1-184 and/or O1-236 polynucleotide sequences may be inserted into a recombinant expression vector. The term recombinant expression vectors refers to a plasmid, virus or other vehicle known in the art that has been manipulated by insertion or incorporation of the O1-180, O1-184 or O1-236 genetic sequences. Such expression vectors contain a promoter sequence which facilitates the efficient transcription of the inserted genetic sequence of the host. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg et al., 1987), the pMSXND expression vector for expression in mammalian cells (Lee and Nathans, 1988) and baculovirus-derived vectors for expression in insect cells. The DNA segment can be present in the vector operably linked to regulatory elements, for example, a promoter (e.g., T7, metallothionein 1, or polyhedrin promoters). Polynucleotide sequences encoding O1-180, O1-184 or O1-236 can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

A. Selectable Markers

In certain embodiments of the invention, the expression cassette and/or constructs of the present invention contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as herpes simplex virus thymidine kinase (tk) are employed. Immunologic markers also can be employed. The selectable marker employed is not believed to be important, so long as it is capable of being expressed simultaneously with the nucleic acid encoding a gene product. Further examples of selectable markers are well known to one of skill in the art and include reporters such as EGFP, βgal or chloramphenicol acetyltransferase (CAT).

B. Control Regions

1. Promoters

The particular promoter employed to control the expression of a polynucleotide sequence of interest is not believed to be important, so long as it is capable of directing the expression of the polynucleotide in the targeted cell. Thus, where a human cell is targeted, it is preferable to position the polynucleotide sequence coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it is desirable to prohibit or reduce expression of one or more of the transgenes. Examples of transgenes that are toxic to the producer cell line are pro-apoptotic and cytokine genes. Several inducible promoter systems are available for production of viral vectors where the transgene product is toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the patient, the gene expression would be constitutively on.

Viral promoters with varying strengths of activity can be utilized depending on the level of expression desired. In mammalian cells, the CMV immediate early promoter is often used to provide strong transcriptional activation. Modified versions of the CMV promoter that are less potent have also been used when reduced levels of expression of the transgene are desired. When expression of a transgene in cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

Similarly tissue specific promoters are used to effect transcription in specific tissues or cells so as to reduce potential toxicity or undesirable effects to non-targeted tissues. For example, promoters such as an oocyte-specific promoter: Zp3 promoter (Lira et al., 1990), a spermatocyte-specific promoter: PGK2 promoter (Zhang et al., 1999); and a spermatid-specific promoter: Protamine promoter (Peschon et al., 1987).

In certain indications, it is desirable to activate transcription at specific times after administration of the vector. This is done with such promoters as those that are hormone or cytokine regulatable. Cytokine and inflammatory protein responsive promoters that can be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It is envisioned that any of the above promoters alone or in combination with another can be useful according to the present invention depending on the action desired. In addition, this list of promoters should not be construed to be exhaustive or limiting, those of skill in the art will know of other promoters that are used in conjunction with the promoters and methods disclosed herein.

2. Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Any promoter/enhancer combination (as per the Eukaryotic Promoter Data Base EPDB) can be used to drive expression of the gene. Eukaryotic cells can support cytoplasmic transcription from certain bacterial promoters if the appropriate bacterial polymerase is provided, either as part of the delivery complex or as an additional genetic expression construct.

3. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence is employed such as human or bovine growth hormone and SV40 polyadenylation signals. Also contemplated as an element of the expression cassette is a terminator. These elements can serve to enhance message levels and to minimize read through from the cassette into other sequences.

4. Integration Sequences

In instances wherein it is beneficial that the expression vector replicate in a cell, the vector may integrate into the genome of the cell by way of integration sequences, i.e., retrovirus long terminal repeat sequences (LTRs), the adeno-associated virus ITR sequences, which are present in the vector, or alternatively, the vector may itself comprise an origin of DNA replication and other sequence which facilitate replication of the vector in the cell while the vector maintains an episomal form. For example, the expression vector may optionally comprise an Epstein-Barr virus (EBV) origin of DNA replication and sequences which encode the EBV EBNA-1 protein in order that episomal replication of the vector is facilitated in a cell into which the vector is introduced. For example, DNA constructs having the EBV origin and the nuclear antigen EBNA-1 coding are capable of replication to high copy number in mammalian cells and are commercially available from, for example, Invitrogen (San Diego, Calif.).

It is important to note that in the present invention it is not necessary for the expression vector to be integrated into the genome of the cell for proper protein expression. Rather, the expression vector may also be present in a desired cell in the form of an episomal molecule. For example, there are certain cell types in which it is not necessary that the expression vector replicate in order to express the desired protein. These cells are those which do not normally replicate and yet are fully capable of gene expression. An expression vector is introduced into non-dividing cells and express the protein encoded thereby in the absence of replication of the expression vector.

C. Methods of Gene Transfer

In order to mediate the effect of the transgene expression in a cell, it will be necessary to transfer the expression constructs of the present invention into a cell. Such transfer may employ viral or non-viral methods of gene transfer. This section provides a discussion of methods and compositions of gene transfer. Still further, one of skill in the art is aware that isolation and purification of microbial expressed polypeptide, or fragments thereof, provided by the invention, may be carried out by conventional means inducing preparative chromatography and immunological separations involving monoclonal or polyclonal antibodies.

1. Non-Viral Transfer

Several non-viral methods for the transfer of expression construct into cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

In a specific embodiment of the present invention, the expression construct is complexed to a cationic polymer. Cationic polymers, which are water-soluble complexes, are well known in the art and have been utilized as a delivery system for DNA plasmids. This strategy employs the use of a soluble system, which will convey the DNA into the cells via a receptor-mediated endocytosis (Wu & Wu 1988). One skilled in the art realizes that the complexing nucleic acids with a cationic polymer will help neutralize the negative charge of the nucleic acid allowing increased endocytic uptake.

In a particular embodiment of the invention, the expression construct is entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the β-lactamase gene, Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection. Also included are various commercial approaches involving “lipofection” technology.

In certain embodiments of the invention, the liposome is complexed with a hemagglutinating virus (HVJ). This has been shown to facilitate fusion with the cell membrane and promote cell entry of liposome-encapsulated DNA (Kaneda et al., 1989). In other embodiments, the liposome is complexed or employed in conjunction with nuclear nonhistone chromosomal proteins (HMG-1) (Kato et al., 1991). In yet further embodiments, the liposome is complexed or employed in conjunction with both HVJ and HMG-1. In that such expression constructs have been successfully employed in transfer and expression of nucleic acid in vitro and in vivo, then they are applicable for the present invention.

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also is specifically delivered into a cell type such as prostate, epithelial or tumor cells, by any number of receptor-ligand systems with or without liposomes. For example, the human prostate-specific antigen (Watt et al., 1986) is used as the receptor for mediated delivery of a nucleic acid in prostate tissue.

In another embodiment of the invention, the expression construct may simply consist of naked recombinant DNA or plasmids. Transfer of the construct is performed by any of the methods mentioned above which physically or chemically permeabilize the cell membrane. This is applicable particularly for transfer in vitro, however, it is applied for in vivo use as well. Dubensky et al., (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM also is transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

2. Viral Vector-Mediated Transfer

In certain embodiments, transgene is incorporated into a viral particle to mediate gene transfer to a cell. Typically, the virus simply will be exposed to the appropriate host cell under physiologic conditions, permitting uptake of the virus. The present methods are advantageously employed using a variety of viral vectors, as discussed below.

### a. Adenovirus

Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

### b. Retrovirus

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing a human cDNA, together with the retroviral LTR and Ψ sequences is introduced into this cell line (by calcium phosphate precipitation for example), the Ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

### c. Adeno-Associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al, 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1995; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996).

AAV-mediated efficient gene transfer and expression in the lung has led to clinical trials for the treatment of cystic fibrosis (Carter and Flotte, 1995; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

### d. Other Viral Vectors

Other viral vectors are employed as expression constructs in the present invention. Vectors derived from viruses such as vaccinia virus (Ridgeway, 1988; Baichwal and Sugden, 1986; Coupar et al., 1988) canary pox virus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

### IV. DIAGNOSTIC USES

The term cell-degenerative disorder denotes the loss of any type of cell in the ovary, either directly or indirectly. For example, in the absence of GDF-9, there is a block in the growth of the granulosa cells leading to eventual degeneration (i.e., death) of the oocytes (Dong et al., 1996). This death of the oocyte appears to lead to differentiation of the granulosa cells. In addition, in the absence of GDF-9, no normal thecal cell layer is formed around the follicles. Thus, in the absence of one oocyte-specific protein, GDF-9, there are defects in three different cell lineages, oocytes, granulosa cells, and thecal cells. In a similar way, death or differentiation of these various cell lineages could be affected by absence or misexpression of O1-180, O1-184, or O1-236. Furthermore, absence or misexpression of O1-180, O1-184, or O1-236 could result in defects in the oocyte/egg leading to the inability of the egg to be fertilized by spermatozoa. Alternatively, embryos may not develop or halt development during the early stage of embryogenesis or show defects in fertilization secondary to absence of these oocyte derived factors.

Therefore, O1-180, O1-184 or O1-236 compositions may be employed as a diagnostic or prognostic indicator of infertility in general. More specifically, point mutations, deletions, insertions or regulatory perturbations can be identified. The present invention contemplates further the diagnosis of infertility detecting changes in the levels of O1-180, O1-184 or O1-236 expression.

One embodiment of the instant invention comprises a method for detecting variation in the expression of O1-180, O1-184 or O1-236. This may comprise determining the level of O1-180, O1-184 or O1-236 expressed, or determining specific alterations in the expressed product. In specific embodiments, alterations are detected in the expression of O1-180, O1-184 or O1-236.

The biological sample can be tissue or fluid. Various embodiments include cells from the testes and ovaries. Other embodiments include fluid samples such as vaginal fluid or seminal fluid.

Nucleic acids used are isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA (cDNA). In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintography of radiolabel or fluorescent label or even via a system using electrical or thermal impulse signals (Affymax Technology; Bellus, 1994).

Following detection, one may compare the results seen in a given patient with a statistically significant reference group of normal patients and patients that have been diagnosed with infertility.

It is contemplated that other mutations in the O1-180, O1-184 or O1-236 polynucleotide sequences may be identified in accordance with the present invention by detecting a nucleotide change in particular nucleic acids (U.S. Pat. No. 4,988,617, incorporated herein by reference). A variety of different assays are contemplated in this regard, including but not limited to, fluorescent in situ hybridization (FISH; U.S. Pat. No. 5,633,365 and U.S. Pat. No. 5,665,549, each incorporated herein by reference), direct DNA sequencing, PFGE analysis, Southern or Northern blotting, single-stranded conformation analysis (SSCA), RNAse protection assay, allele-specific oligonucleotide (ASO) (e.g., U.S. Pat. No. 5,639,611), dot blot analysis, denaturing gradient gel electrophoresis (e.g., U.S. Pat. No. 5,190,856 incorporated herein by reference), RFLP (e.g., U.S. Pat. No. 5,324,631 incorporated herein by reference) and PCR™-SSCP. Methods for detecting and quantitating gene sequences, such as mutated genes and oncogenes, in for example biological fluids are described in U.S. Pat. No. 5,496,699, incorporated herein by reference.

Yet further, it is contemplated by that chip-based DNA technologies such as those described by Hacia et al. (1996) and Shoemaker et al. (1996) can be used for diagnosis of infertility. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al., (1994); Fodor et al., (1991).

Antibodies can be used in characterizing the O1-180, O1-184 or O1-236 content through techniques such as ELISAs and Western blot analysis. This may provide a prenatal screen or in counseling for those individuals seeking to have children.

The steps of various other useful immunodetection methods have been described in the scientific literature, such as, e.g., Nakamura et al., (1987). Immunoassays, in their most simple and direct sense, are binding assays. Certain preferred immunoassays are the various types of radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

The antibodies of the invention can be bound to many different carriers and used to detect the presence of an antigen comprising the polypeptide of the invention. Samples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the antibody, or will be able to ascertain such, using routine experimentation.

Another technique which may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, puridoxal, and fluorescein, which can react with specific anti-hapten antibodies.

In using the monoclonal antibodies of the invention for the in vivo detection of antigen, the detectably labeled antibody is given a dose which is diagnostically effective. The term diagnostically effective means that the amount of detectably labeled monoclonal antibody is administered in sufficient quantity to enable detection of the site having the antigen composing a polypeptide of the invention for which the monoclonal antibodies are specific. The concentration of detectably labeled monoclonal antibody which is administered should be sufficient such that the binding to those cells having the polypeptide is detectable compared to the background. Further, it is desirable that the detectably labeled monoclonal antibody be rapidly cleared from the circulatory system in order to give the best target-to-background signal ratio. As a rule, the dosage of detectably labeled monoclonal antibody for in vivo diagnosis will vary depending on such factors as age, sex, and extent of disease of the individual. Such dosages may vary, for example, depending on whether multiple injections are given, antigenic burden, and other factors known to those of skill in the art.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that deleterious radiation with respect to the host is minimized. Ideally, a radioisotope used for in vivo imaging will lack a particle emission, but produce a large number of photons in the 140-250 keV range, which may readily be detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are ¹¹¹In, ⁹⁷Ru, ⁶⁷Ga, ⁶⁸ Ga, ⁷² As, ⁸⁹ Zr and ²⁰¹Ti.

The monoclonal antibodies of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include ¹⁵⁷Gd, ⁵⁵Mn, ¹⁶²Dy, ⁵⁵Cr and ⁵⁶Fe.

The term cell-proliferative disorder or hyperproliferative disorder denotes malignant as well as non-malignant cell populations which often appear to differ from the surrounding tissue both morphologically and genotypically. The O1-180, O1-184 and O1-236 polynucleotides that are antisense molecules are useful in treating malignancies of the various organ systems, particularly, for example, the ovaries. Essentially, any disorder which is etiologically linked to altered expression of O1-180, O1-184 or O1-236 could be considered susceptible to treatment with a O1-180, O1-184 or O1-236 suppressing reagent, respectively.

The invention provides a method for detecting a cell proliferative disorder of the ovary which comprises contacting an anti-O1-180, O1-184 or O1-236 antibody with a cell suspected of having an O1-180, O1-184 or O1-236 associated disorder and detecting binding to the antibody. The antibody reactive with O1-180, O1-184 or O1-236 is labeled with a compound which allows detection of binding to O1-180, O1-184 or O1-236, respectively. For purposes of the invention, an antibody specific for an O1-180, O1-184 or O1-236 polypeptide may be used to detect the level of O1-180, O1-184 or O1-236, respectively, in biological fluids and tissues. Any specimen containing a detectable amount of antigen can be used. A preferred sample of this invention is tissue of ovarian origin, specifically tissue containing oocytes or ovarian follicular fluid. The level of O1-180, O1-184 or O1-236 in the suspect cell can be compared with the level in a normal cell to determine whether the subject has an O1-180, O1-184 or O1-236-associated cell proliferative disorder. Preferably the subject is human. The antibodies of the invention can be used in any subject in which, it is desirable to administer in vitro or in vivo immunodiagnosis or immunotherapy. The antibodies of the invention are suited for use, for example, in immuno assays in which they can be utilized in liquid phase or bound to a solid phase carrier. In addition, the antibodies in these immunoassays can be detectably labeled in various ways. Examples of types of immunoassays which can utilize antibodies of the invention are competitive and non-competitive immunoassays in either a direct or indirect format. Examples of such immunoassays are the radioimmunoassay (RIA) and the sandwich (ELISA) assay. Detection of the antigens using the antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

### V. THERAPEUTIC USES

Due to the expression of O1-180, O1-184 and O1-236 in the reproductive tract, there are a variety of applications using the polypeptides, polynucleotides and antibodies of the invention, related to contraception, fertility and pregnancy. O1-180, O1-184 and O1-236 could play a role in regulation of the menstrual cycle and, therefore, could be useful in various contraceptive regimens.

It is also contemplated that O1-180, O1-184, or O1-236 polynucleotide sequences, polypeptide sequences, antibodies, fragments thereof or mutants thereof may be used to inhibit or enhance early embryogenesis by distrubing the maternal genome. One of skill in the art is aware that disruptions of the maternal genome that cause phenotypes in embryonic development are termed maternal effect mutations. Two such examples have been characterized in mice using knockout technology. In each example, the gene product is normally accumulated in growing oocytes and persists in the early developing embryo and the phenotype affects offspring of knockout females, regardless of their genotype or gender. The first identified gene encodes MATER (maternal antigen that embryos require), which is necessary for development beyond the two-cell stage and has been implicated in establishing embryonic genome transcription patterns (Tong et al., 2000). The second identified gene encodes DNMT1o, an oocyte-specific DNA methyltransferase critical for maintaining imprinting patterns established in the embryonic genome and the viability of the developing mouse during the last third of gestation (Howell et al., 2001). Presumably many other oocyte-derived factors mediate the complexities of early embryogenesis, thus, it is contemplated that the O1-180 and O1-236 are maternal effect genes since they function in processes of early embryogenesis.

In further embodiments, it is contemplated that O1-236 may play a role in in chromatin remodeling during early embryoonic development. For example, studies have predicted the presence of a mammalian nuclear protein that is necessary for oocyte remodeling of sperm DNA, and is released into the ooplasm at germinal vesicle breakdown (Maeda et al., 1998). Yet further, it is known that oocytes can efficiently remodel not only sperm nuclei during fertilization, but also somatic cell nuclei. Thus, the inventors have contemplated the role of NPM2 in nuclear transfer cloning (Zuccotti et al., 2000). It envisioned that NPM2 (encoded by O1-236) is a critical factor in mammalian oocytes for chromatin remodeling during early embryonic development. Thus, supplementing enucleated oocytes with NPM2 may facilitate cloning by nuclear transfer technologies.

The monoclonal antibodies of the invention can be used in vitro and in vivo to monitor the course of amelioration of an O1-180, O1-184 or O1-236-associated disease in a subject. Thus, for example, by measuring the increase or decrease in the number of cells expressing antigen comprising a polypeptide of the invention or changes in the concentration of such antigen present in various body fluids, it would be possible to determine whether a particular therapeutic regimen aimed at ameliorating the O1-180, O1-184 or O1-236-associated disease is effective. The term ameliorate denotes a lessening of the detrimental effect of the O1-180, O1-184 or O1-236-associated disease in the subject receiving therapy.

The present invention identifies nucleotide sequences that can be expressed in an altered manner as compared to expression in a normal cell, therefore, it is possible to design appropriate therapeutic or diagnostic techniques directed to this sequence. Thus, where a cell-proliferative disorder is associated with the expression of O1-180, O1-184 or O1-236, nucleic acid sequences that interfere with the expression of O1-180, O1-184 or O1-236, respectively, at the translational level can be used. This approach utilizes, for example, antisense nucleic acids or ribozymes to block translation of a specific O1-180, O1-184 or O1-236 mRNA, either by masking that mRNA with an antisense nucleic acid or by cleaving it with a ribozyme.

Antisense nucleic acids are DNA or RNA molecules that are complementary to at least a portion of a specific mRNA molecule (Weintraub, 1990). In the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target O1-180, O1-184 or O1-236-producing cell. The use of antisense methods to inhibit the in vitro translation of genes is well known in the art (Marcus-Sakura, 1988).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, 1988). A major advantage of this approach is that, because they are sequence-specific, only mRNAs with particular sequences are inactivated.

There are two basic types of ribozymes namely, tetrahymena-type (Hasselhoff, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-based recognition sequences are preferable to shorter recognition sequences.

It is also contemplated in the present invention that double-stranded RNA is used as an interference molecule, e.g., RNA interference (RNAi). RNA interference is used to “knock down” or inhibit a particular gene of interest by simply injecting, bathing or feeding to the organism of interest the double-stranded RNA molecule. This technique selectively “knock downs” gene function without requiring transfection or recombinant techniques (Giet, 2001; Hammond, 2001; Stein P, et al., 2002; Svoboda P, et al., 2001; Svoboda P, et al., 2000). Thus, in certain embodiments, double-stranded O1-180, O1-184 or O1-236 RNA is synthesized or produced using standard molecular techniques described herein.

The present invention also provides gene therapy for the treatment of cell proliferative or degenerative disorders which are mediated by O1-180, O1-184 or O1-236 proteins. Such therapy would achieve its therapeutic effect by introduction of the respective O1-180, O1-184 or O1-236 cDNAs or O1-180, O1-184, or O1-236 antisense polynucleotide into cells having the proliferative or degenerative disorder. Delivery of O1-180, O1-184, or O1-236 cDNAs or antisense O1-180, O1-184 or O1-236 polynucleotides can be achieved using a recombinant expression vector such as a chimeric virus or a colloidal dispersion system. Especially preferred for therapeutic delivery of cDNAs or antisense sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting an O1-180, O1-184 or O1-236 sequence of interest into the viral vector, along with another gene which encodes the ligand for a receptor on a specific target cell, for example, the vector is now target specific. Retroviral vectors can be made target specific by inserting, for example, a polynucleotide encoding a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing an O1-180, O1-184 or O1-236 cDNA or O1-180, O1-184, or O1-236 antisense polynucleotides.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packing mechanism to recognize an RNA transcript for encapsidation. Helper cell lines which have deletions of the packaging signal include, but are not limited to ψ2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for O1-180, O1-184 or O1-236 cDNAs or O1-180, O1-184, or O1-236 antisense polynucleotides is a colloidal dispersion system. Colloidal dispersion systems include macromolecule complexes, nanocapsules complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley et al., 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high exigency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Manning et al., 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

### VI. SCREENING FOR MODULATORS

As used herein, the term “candidate substance” refers to any molecule that may potentially modulate O1-180, O1-184 or O1-236 activity, expression or function. Candidate compounds may include fragments or parts of naturally-occurring compounds or may be found as active combinations of known compounds which are otherwise inactive. The candidate substance can be a polynucleotide, a polypeptide, a small molecule, etc. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

In specific embodiments, a small molecule library that is created by chemical genetics may be screened to identify a candidate substance that may be a modulator of the present invention (Schreiber et al., 2001a; Schreiber et al., 2001b). Chemical genetics is the technology that uses small molecules to modulate the functions of proteins rapidly and conditionally. The basic approach requires identification of compounds that regulate pathways and bind to proteins with high specificity. Small molecules are prepared using diversity-oriented synthesis, and the split-pool strategy to allow spatial segregation on individual polymer beads. Each bead contains compounds to generate a stock solution that can be used for many biological assays.

The goal of rational drug design is to produce structural analogs of biologically active target compounds. By creating such analogs, it is possible to fashion drugs which are more active or stable than the natural molecules, which have different susceptibility to alteration or which may affect the function of various other molecules. In one approach, one would generate a three-dimensional structure for a molecule like O1-180, O1-184 or O1-236 polypeptide, and then design a molecule for its ability to interact with O1-180, O1-184 or O1-236 polypeptide. This could be accomplished by X-ray crystallography, computer modeling or by a combination of both approaches. The same approach may be applied to identifying interacting molecules of O1-180, O1-184 or O1-236 polypeptides and/or polynucleotides.

It also is possible to use antibodies to ascertain the structure of a target compound or activator. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

It will, of course, be understood that all the screening methods of the present invention are useful in themselves notwithstanding the fact that effective candidates may not be found. The invention provides methods for screening for such candidates, not solely methods of finding them.

Thus, the present invention contemplates the use of O1-180, O1-184 or O1-236 and active fragments, and nucleic acids coding therefore, in the screening of compounds for activity in either stimulating O1-180, O1-184 or O1-236, overcoming the lack of O1-180, O1-184 or O1-236 or blocking or inhibiting the effect of an O1-180, O1-184 or O1-236 molecule. These assays may make use of a variety of different formats and may depend on the kind of “activity” for which the screen is being conducted.

In one embodiment, the invention is to be applied for the screening of compounds that bind to the O1-180, O1-184 or O1-236 polypeptide or fragment thereof. The polypeptide or fragment may be either free in solution, fixed to a support, expressed in or on the surface of a cell. Either the polypeptide or the compound may be labeled, thereby permitting the determination of binding.

In another embodiment, the assay may measure the inhibition of binding of O1-180, O1-184 or O1-236 to a natural or artificial substrate or binding partner. Competitive binding assays can be performed in which one of the agents (O1-180, O1-184 or O1-236, binding partner or compound) is labeled. Usually, the polypeptide will be the labeled species. One may measure the amount of free label versus bound label to determine binding or inhibition of binding.

Another technique for high throughput screening of compounds is described in WO 84/03564. Large numbers of small peptide test compounds are synthesized on a solid substrate, such as plastic pins or some other surface. The peptide test compounds are reacted with O1-180, O1-184 or O1-236 and washed. Bound polypeptide is detected by various methods.

Purified O1-180, O1-184 or O1-236 can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the O1-180, O1-184 or O1-236 active region to a solid phase.

Various cell lines containing wild-type or natural or engineered mutations in O1-180, O1-184 or O1-236 gene can be used to study various functional attributes of O1-180, O1-184 or O1-236 and how a candidate compound affects these attributes. Methods for engineering mutations are described elsewhere in this document, as are naturally-occurring mutations in O1-180, O1-184 or O1-236 that lead to, contribute to and/or otherwise cause infertility. In such assays, the compound would be formulated appropriately, given its biochemical nature, and contacted with a target cell. Depending on the assay, cell culture may be required. The cell may then be examined by virtue of a number of different physiologic assays. Alternatively, molecular analysis may be performed in which the function of O1-180, O1-184 or O1-236, or related pathways, may be explored.

In a specific embodiment, yeast two-hybrid analysis is performed by standard means in the art with the polypeptides of the present invention, i.e., O1-180, O1-184 or O1-236. Two hybrid screen is used to elucidate or characterize the function of a protein by identifying other proteins with which it interacts. The protein of unknown function, herein referred to as the “bait” is produced as a chimeric protein additionally containing the DNA binding domain of GAL4. Plasmids containing nucleotide sequences which express this chimeric protein are transformed into yeast cells, which also contain a representative plasmid from a library containing the GAL4 activation domain fused to different nucleotide sequences encoding different potential target proteins. If the bait protein physically interacts with a target protein, the GAL4 activation domain and GAL4 DNA binding domain are tethered and are thereby able to act conjunctively to promote transcription of a reporter gene. If no interaction occurs between the bait protein and the potential target protein in a particular cell, the GAL4 components remain separate and unable to promote reporter gene transcription on their own. One skilled in the art is aware that different reporter genes can be utilized, including β-galactosidase, HIS3, ADE2, or URA3. Furthermore, multiple reporter sequences, each under the control of a different inducible promoter, can be utilized within the same cell to indicate interaction of the GAL4 components (and thus a specific bait and target protein). A skilled artisan is aware that use of multiple reporter sequences decreases the chances of obtaining false positive candidates. Also, alternative DNA-binding domain/activation domain components may be used, such as LexA. One skilled in the art is aware that any activation domain may be paired with any DNA binding domain so long as they are able to generate transactivation of a reporter gene. Furthermore, a skilled artisan is aware that either of the two components may be of prokaryotic origin, as long as the other component is present and they jointly allow transactivation of the reporter gene, as with the LexA system.

Two hybrid experimental reagents and design are well known to those skilled in the art (see The Yeast Two-Hybrid System by P. L. Bartel and S. Fields (eds.) (Oxford University Press, 1997), including the most updated improvements of the system (Fashena et al., 2000). A skilled artisan is aware of commercially available vectors, such as the Matchmaker™ Systems from Clontech (Palo Alto, Calif.) or the HybriZAP® 2.1 Two Hybrid System (Stratagene; La Jolla, Calif.), or vectors available through the research community (Yang et al., 1995; James et al., 1996). In alternative embodiments, organisms other than yeast are used for two hybrid analysis, such as mammals (Mammalian Two Hybrid Assay Kit from Stratagene (La Jolla, Calif.)) or E. coli (Hu et al., 2000).

In an alternative embodiment, a two hybrid system is utilized wherein protein-protein interactions are detected in a cytoplasmic-based assay. In this embodiment, proteins are expressed in the cytoplasm, which allows posttranslational modifications to occur and permits transcriptional activators and inhibitors to be used as bait in the screen. An example of such a system is the CytoTrap® Two-Hybrid System from Stratagene (La Jolla, Calif.), in which a target protein becomes anchored to a cell membrane of a yeast which contains a temperature sensitive mutation in the cdc25 gene, the yeast homologue for hSos (a guanyl nucleotide exchange factor). Upon binding of a bait protein to the target, hSos is localized to the membrane, which allows activation of RAS by promoting GDP/GTP exchange. RAS then activates a signaling cascade which allows growth at 37° C. of a mutant yeast cdc25H. Vectors (such as pMyr and pSos) and other experimental details are available for this system to a skilled artisan through Stratagene (La Jolla, Calif.). (See also, for example, U.S. Pat. No. 5,776,689, herein incorporated by reference).

Thus, in accordance with an embodiment of the present invention, there is a method of screening for a peptide which interacts with O1-180, O1-184 or O1-236 comprising introducing into a cell a first nucleic acid comprising a DNA segment encoding a test peptide, wherein the test peptide is fused to a DNA binding domain, and a second nucleic acid comprising a DNA segment encoding at least part of O1-180, O1-184 or O1-236, respectively, wherein at least part of O1-180, O1-184 or O1-236 respectively, is fused to a DNA activation domain. Subsequently, there is an assay for interaction between the test peptide and the O1-180, O1-184 or O1-236 polypeptide or fragment thereof by assaying for interaction between the DNA binding domain and the DNA activation domain. For example, the assay for interaction between the DNA binding and activation domains may be activation of expression of β-galactosidase.

An alternative method is screening of lambda.gt11, lambda.LZAP (Stratagene) or equivalent cDNA expression libraries with recombinant O1-180, O1-184 or O1-236. Recombinant O1-180, O1-184 or O1-236 or fragments thereof are fused to small peptide tags such as FLAG, HSV or GST. The peptide tags can possess convenient phosphorylation sites for a kinase such as heart muscle creatine kinase or they can be biotinylated. Recombinant O1-180, O1-184 or O1-236 can be phosphorylated with ³²[P] or used unlabeled and detected with streptavidin or antibodies against the tags lambdagt11cDNA expression libraries are made from cells of interest and are incubated with the recombinant O1-180, O1-184 or O1-236, washed and cDNA clones which interact with O1-180, O1-184 or O1-236 isolated. Such methods are routinely used by skilled artisans. See, e.g., Sambrook (supra).

Another method is the screening of a mammalian expression library in which the cDNAs are cloned into a vector between a mammalian promoter and polyadenylation site and transiently transfected in cells. Forty-eight hours later the binding protein is detected by incubation of fixed and washed cells with a labeled O1-180, O1-184 or O1-236. In this manner, pools of cDNAs containing the cDNA encoding the binding protein of interest can be selected and the cDNA of interest can be isolated by further subdivision of each pool followed by cycles of transient transfection, binding and autoradiography. Alternatively, the cDNA of interest can be isolated by transfecting the entire cDNA library into mammalian cells and panning the cells on a dish containing the O1-180, O1-184 or O1-236 bound to the plate. Cells which attach after washing are lysed and the plasmid DNA isolated, amplified in bacteria, and the cycle of transfection and panning repeated until a single cDNA clone is obtained. See Seed et al., 1987 and Aruffo et al., 1987 which are herein incorporated by reference. If the binding protein is secreted, its cDNA can be obtained by a similar pooling strategy once a binding or neutralizing assay has been established for assaying supernatants from transiently transfected cells. General methods for screening supernatants are disclosed in Wong et al., (1985).

Another alternative method is the isolation of proteins interacting with the O1-180, O1-184 or O1-236 directly from cells. Fusion proteins of O1-180, O1-184 or O1-236 with GST or small peptide tags are made and immobilized on beads. Biosynthetically labeled or unlabeled protein extracts from the cells of interest are prepared, incubated with the beads and washed with buffer. Proteins interacting with the O1-180, O1-184 or O1-236 are eluted specifically from the beads and analyzed by SDS-PAGE. Binding partner primary amino acid sequence data are obtained by microsequencing. Optionally, the cells can be treated with agents that induce a functional response such as tyrosine phosphorylation of cellular proteins. An example of such an agent would be a growth factor or cytokine such as interleukin-2.

Another alternative method is immunoaffinity purification. Recombinant O1-180, O1-184 or O1-236 is incubated with labeled or unlabeled cell extracts and immunoprecipitated with anti-O1-180, O1-184 or O1-236 antibodies. The immunoprecipitate is recovered with protein A-Sepharose and analyzed by SDS-PAGE. Unlabelled proteins are labeled by biotinylation and detected on SDS gels with streptavidin. Binding partner proteins are analyzed by microsequencing. Further, standard biochemical purification steps known to those skilled in the art may be used prior to microsequencing.

Yet another alternative method is screening of peptide libraries for binding partners. Recombinant tagged or labeled O1-180, O1-184 or O1-236 is used to select peptides from a peptide or phosphopeptide library which interact with the O1-180, O1-184 or O1-236. Sequencing of the peptides leads to identification of consensus peptide sequences which might be found in interacting proteins.

The present invention also encompasses the use of various animal models. Thus, any identity seen between human and other animal O1-180, O1-184 or O1-236 provides an excellent opportunity to examine the function of O1-180, O1-184 or O1-236 in a whole animal system where it is normally expressed. By developing or isolating mutant cells lines that fail to express normal O1-180, O1-184 or O1-236, one can generate models in mice that enable one to study the mechanism of O1-180, O1-184 or O1-236 and its role in oogenesis and embryonic development.

Treatment of animals with test compounds will involve the administration of the compound, in an appropriate form, to the animal. Administration will be by any route that could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, increased fertility, decreased fertility or contraception.

In one embodiment of the invention, transgenic animals are produced which contain a functional transgene encoding a functional O1-180, O1-184 or O1-236 polypeptide or variants thereof. Transgenic animals expressing O1-180, O1-184 or O1-236 transgenes, recombinant cell lines derived from such animals and transgenic embryos may be useful in methods for screening for and identifying agents that induce or repress function of O1-180, O1-184 or O1-236. Transgenic animals of the present invention also can be used as models for studying disease states.

In one embodiment of the invention, an O1-180, O1-184 or O1-236 transgene is introduced into a non-human host to produce a transgenic animal expressing an O1-180, O1-184 or O1-236. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al., 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety). Expression of the transgene may be regulatable by incorporating sequences such as cytokine or hormone response elements. This is done with such promoters as those that are hormone or cytokine regulatable. Cytokine and inflammatory protein responsive promoters that can be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It may be desirable to replace the endogenous O1-180, O1-184 or O1-236 by homologous recombination between the transgene and the endogenous gene; or the endogenous gene may be eliminated by deletion as in the preparation of “knock-out” animals. Typcially, targeting vectors that contain a portion of the gene of interest and a selection marker are generated and transfected into embryonic stem (ES) cells. These targeting vectors are electroporated into the hprt-negative ES cell line and selected in HAT and FIAU. ES cells with the correct mutation are injected into blastocysts to generate chimeras and eventually heterozygotes and homozygotes for the mutant O1-180, O1-184 and O1-236 genes. Thus, the absence of O1-180, O1-184 or O1-236 in “knock-out” mice permits the study of the effects that loss of O1-180, O1-184 or O1-236 protein has on a cell in vivo.

As noted above, transgenic animals and cell lines derived from such animals may find use in certain testing experiments. In this regard, transgenic animals and cell lines capable of expressing wild-type or mutant O1-180, O1-184 or O1-236 may be exposed to test substances. These test substances can be screened for the ability to enhance wild-type O1-180, O1-184 or O1-236 expression and or function or impair the expression or function of mutant O1-180, O1-184 or O1-236.

### VII. FORMULATIONS AND ROUTES FOR ADMINISTRATION TO PATIENTS

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Creation of a cDNA Subtractive Hybridization Library

Ovaries from Gdf9 knockout mice are histologically very different from wild-type ovaries due to the early block in folliculogenesis. In particular, one layer primary follicles are relatively enriched in Gdf9 knockout ovaries and abnormal follicular nests are formed after oocyte loss. The inventors took advantage of these differences in ovary composition and related them to alterations in gene expression patterns to clone novel ovary-expressed transcripts which are upregulated in the Gdf9 knockout ovaries.

Ovaries from either Gdf9 knockout mice (C57BL/6/129SvEv hybrid) or wild-type mice were collected and polyA+ mRNA was made from each pool. Using a modified version of the CLONTECH PCR-Select Subtraction kit, the inventors generated a pBluescript SK+plasmid-based cDNA library which was expected to be enriched for sequences upregulated in the Gdf9 knockout ovaries.

Ligations into the NotI site of pBluescript SK+ were performed with a low molar ratio of EagI-digested cDNA fragment inserts to vector to prevent multiple inserts into the vector. Transformations were performed, and >1000 independent bacterial clones were picked and stored in glycerol at −80° C. The remainder of the ligation mix was stored at −80° C. for future transformations.

### Example 2

### Initial Sequence Analysis of pOvary1 (pO1) Library Inserts

The inventors performed sequence analysis of 331 inserts from the pO1 subtractive hybridization of cDNA library. An Applied Biosystems 373 DNA Sequencer was used to sequence these clones. BLAST searches were performed using the National Center for Biotechnology Information databases. Novel sequences were analyzed for open reading frames and compared to previously identified novel sequences using DNASTAR analysis programs. A summary of the data is presented in Table 1. As shown, the majority of the clones were known genes or matched mouse or human ESTs. 9.4% of the clones failed to match any known sequence in the database.

TABLE 1
Summary of database searches of pO1 cDNA clones

| pO1 cDNA Matches | Number identified | Percentage |
|---|---|---|
| Known Genes | 180 | 54.4% |
| Mouse/Human EST | 120 | 36.2% |
| RARE ESTs (1 EST match) | (8) | (2.4%) |
| ESTs from 2-cell library | (3) | (0.9%) |
| No match | 31 | 9.4% |
| Total | 331 | 100% |

### Example 3

### Northern Blot Analysis

Northern blot analysis was performed using standard techniques well known and used in the art. Briefly, total RNA from tissues was obtained by the RNA STAT-60 method (Leedo Medical Laboratories, Inc.). RNA was isolated from the following tissues: ovaries, brain, lung, heart, stomach, spleen, liver, small intestine, kidney, testes, uterus, colon, prostate, placenta, pancreas, and muscle. Agarose gel electrophoresis of RNA, transfer to nylon membranes, and subsequent hybridization were performed by standard methods (Sambrook et al., 1989).

### Example 4

### In Situ Hybridization

In situ hybridization of ovaries was performed as described previously (Albrecht et al., 1997; Elvin et al., 1999) using partial Npm2, Npm2, or Npm3, Zar1, or O1-184 cDNA fragments. The “sense”probe revealed no hybridization (data not shown). Briefly, the cDNA fragments in pBluescript SK+ or T-vector (Promega, Madison, Wis.) served as templates for generating sense and antisense strands with [³⁵S]-dUTP using the Riboprobe T7/SP6 combination system (Promega). Sections were exposed to photographic emulsion for 4-7 days at 4° C. After the slides were developed and fixed, they were counterstained with hematoxylin.

### Example 5

### Oocyte Collection and Embryo Culture

To collect non-SN (surrounded nucleolus) configuration oocytes, ovaries of 10 day old mice were digested with collagenase as described (Eppig, 1978).

For GV-stage oocytes, adult females were injected intraperitoneally with 5 IU PMSG (pregnant mare serum gonadotropin) and oocytes were recovered 46 hours later by large follicle puncture.

For metaphase II oocytes or in vivo fertilized embryos, females were treated with 5 IU PMSG followed by 5 IU hCG (human chorionic gonadotropin) to induce superovulation as described (Hogan et al., 1994). For fertilization, females were mated overnight with stud males and vaginal plugs checked the following morning. Mature metaphase II oocytes and 1-cell embryos were recovered from oviducts 18-24 hours post-hCG treatment in M2 media (Sigma, St. Louis, Mo.) with hyaluronidase. Collections of embryos 45, 55, and 72 hours post-hCG were accomplished by flushing oviducts with M2 media. For 24 hour culture experiments, eggs and embryos were kept under 5% CO₂ in M16 media (Sigma) until the transition to blastocyst stage when they were transferred to M15. For one experiment, colcemid was added to the media to arrest cells in mitosis (Sigma #D1925; 160 ng/mL).

For in vitro fertilization, sexually mature mice were injected with 5 IU of PMSG, cumulus-enclosed oocyte complexes were isolated 46 hours later, and cultured for 17 hours in minimal essential medium with 5% serum. Mature MII-stage eggs were mixed with capacitated sperm from wild-type (C57BL/6J×SJL/J) F1 mice as described (Eppig, 1999). The development of zygotes and two-cell-stage embryos were assessed at 6 and 24 h after fertilization, respectively.

### Example 6

### Expression Analysis and cDNA Screening of Ovarian-Expressed Genes

Northern blot analysis was performed on all cDNAs which failed to match sequences in any database. Additionally, sequences matching ESTs derived predominantly from mouse 2-cell embryo cDNA libraries (e.g., Zar1, O1-184, and Npm2) were analyzed. The rationale for analyzing this last group of ESTs was that mRNAs expressed at high levels in oocytes may persist until the 2-cell stage and may play a role in early embryonic development including fertilization of the egg or fusion of the male and female pronuclei.

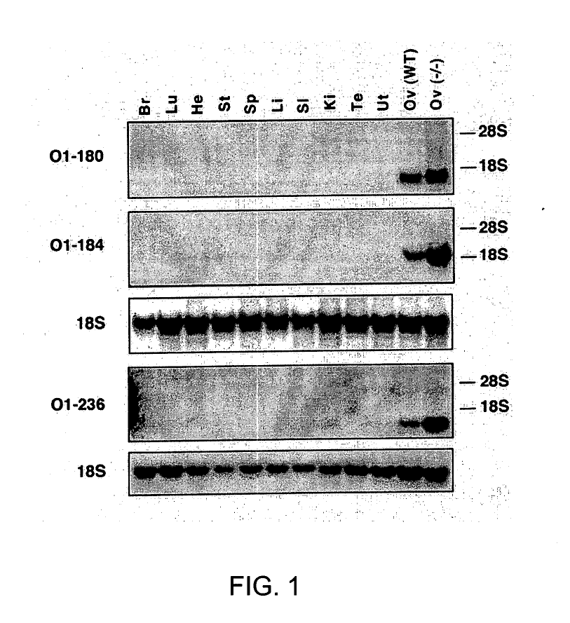

The results of the initial screen of novel ovarian genes is presented in Table 2. Northern blot analysis of 23 clones demonstrated that 8 of these clones were upregulated in the Gdf9 knockout ovary indicating that the subtractive hybridization protocol used was adequate. Northern blot analysis using total RNA isolated from either adult C57BL/6/129SvEv hybrid mice (the ovarian RNA) or Swiss WEBSTER mice (all other tissues) also demonstrated that four of these clones including 2 clones which matched ESTs sequenced from 2-cell libraries were only expressed in the ovary (FIG. 1). The O1-236 fragment probe (749 bp) detected a transcript of approximately 1.0 kb (FIG. 1). Several clones have so far been analyzed for their ovarian localization by in situ hybridization analysis (FIG. 2). Clones O1-180 (herein after referred to as Zar1), O1-184, and O1-236 (herein after referred to as Npm2) were oocyte-specific and expressed in oocytes of primary (one-layer) preantral follicles through ovulation (FIG. 2).

TABLE 2
Analysis of ovarian cDNAs with no known function

| | | | | Further studies |
|---|---|---|---|---|
| | | | | (in situ |
| | Adult | Upregulated in | | hybridization; |
| PO1 | mRNA | Gdf9 knockout | Database | chromosomal |
| cDNA | Expression | ovary | match | mapping) |
| 24 | Multiple | No | — | No |
| 27 | Multiple | Yes | — | Oocyte-specific |
| | | | | by in situ |
| 37 | Multiple | Yes | — | No |
| 70 | Multiple | No | — | No |
| 91 | | | 1 EST (2-cell) | |
| 97 | Multiple | No | ? | No |
| 101 | Multiple | No1 | — | No |
| 114 | Multiple | No | — | No |
| 110 | Multiple | Yes | — | No |
| 126 | Multiple | Yes | — | No |
| 180 | Ovary- | Yes | — | Oocyte-specific |
| (Zar1) | specific | | | by in situ |
| 184 | Ovary- | Yes | >1 EST (All 2- | Oocyte-specific |
| | specific | | cell) | by in situ |
| 186 | Ovary- | Yes | — | Granulosa cell- |
| | specific | | | specific by |
| | | | | in situ |
| 223 | Multiple | No | — | No |
| 224 | Multiple | No | — | No |
| 236 | Ovary- | Yes | 6 EST (2 c-cell | Oocyte-specific |
| (Npm2) | specific | | and others) | by in situ |
| 255 | Multiple | No | “zinc-finger” | |
| | | | domains | |
| 279 | Multiple | No | — | No |
| 317 | Multiple | No | — | No |
| 330 | Multiple | No | — | No |
| 331 | Multiple | No | — | No |
| 332 | Multiple | No | — | No |
| 334 | Multiple | No | — | No |
| 371 | Multiple | No | — | No |

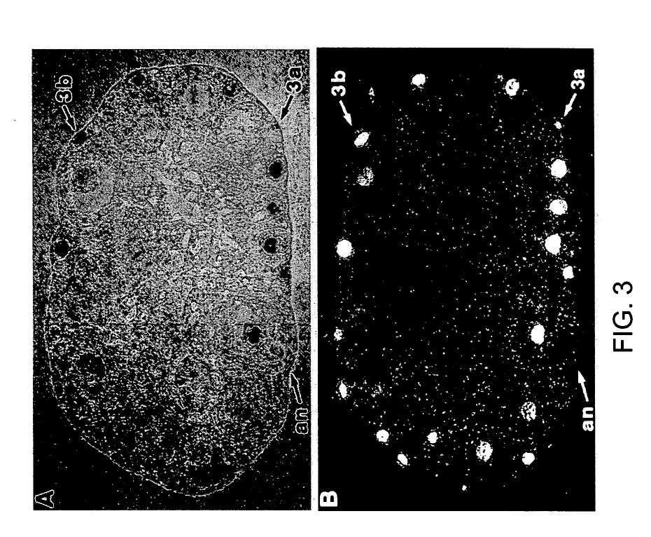

The O1-236 gene product was oocyte-specific (FIG. 3). O1-236 was not expressed in oocytes of primordial (type 2) or small type 3a follicles (Pedersen et al., 1968), but was first detected in oocytes of intermediate-size type 3a follicles and all type 3b follicles (i.e., follicles with >20 granulosa cells surrounding the oocyte in largest cross-section). Expression of the O1-236 mRNA persisted through the antral follicle stage. Interestingly, the oocyte-specific expression pattern of the O1-236 gene product paralleled the expression of other oocyte-specific genes which the inventors have studied including Gdf9 (McGrath et al., 1995) and bone morphogenetic protein (Dube et al., 1998).

### Example 7

### Cloning of Mouse Npm2

Wild-type ovary and Gdf9 knockout ZAP Express ovary cDNA libraries were synthesized and were screened to isolate full-length cDNAs for the above-mentioned three clones. Each full-length cDNA was again subjected to database searches and analyzed for an open reading frame, initiation ATG, and protein homology. The full-length cDNAs approximate the mRNA sizes determined from Northern blot analysis. Database searches using the predicted amino acid sequence permitted the identification of important domains (e.g., signal peptide sequences, transmembrane domains, zinc fingers, etc.) which were useful to define the possible function and cellular localization of the novel protein.

The O1-236 partial cDNA fragment identified in Example 1 was used to screen Matzuk laboratory ZAP Express (Stratagene) ovarian cDNA libraries generated from either wild-type or Gdf9 deficient ovaries (Dube et al., 1998). In brief, approximately 300,000 clones of either wild-type or Gdf9 knockout mouse ovary cDNA libraries were hybridized to [alpha-³²P] dCTP random-primed probes in Church's solution at 63° C. Filters were washed with 0.1× Church's solution and exposed overnight at −80° C.

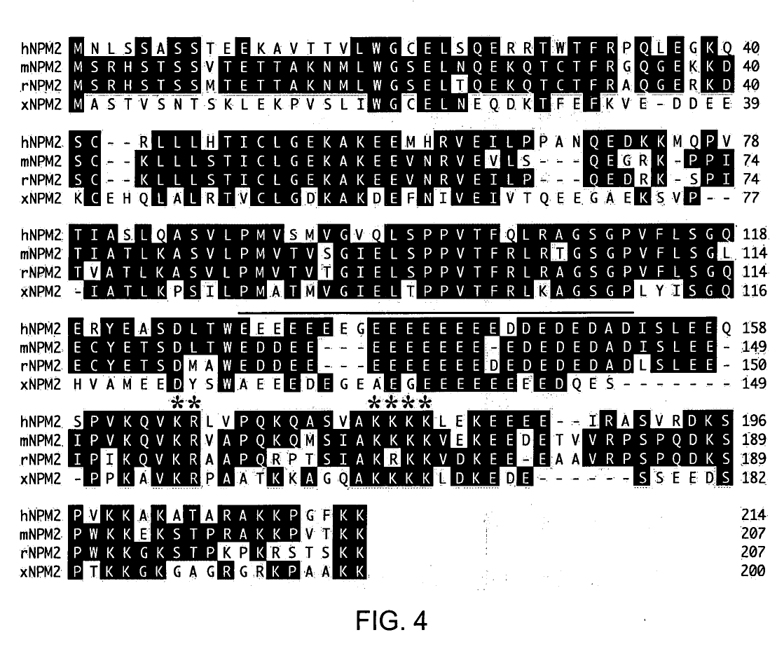

Upon primary screening of the mouse ovarian cDNA libraries, the O1-236 cDNA fragment detected 22 positive phage clones out of 300,000 screened. Two of these clones (236-1 and 236-3), which approximated the mRNA size and which were derived from the two independent libraries, were analyzed further by restriction endonuclease digestion and DNA sequence analysis. These independent clones formed a 984 bp overlapping contig (excluding the polyA sequences) and encoded a 207 amino acid open reading frame. Including the polyA tail, this sequence approximated the 1.0 kb mRNA seen by Northern blot analysis, which suggested that nearly all of the 5′ UTR sequence had been isolated. When the nucleotide sequence was subjected to public database search, no significant matches were derived. However, a database search with the 207 amino acid open reading frame demonstrated high homology with several nucleoplasmin homologs from several species. Interestingly, O1-236 showed highest homology with Xenopus laevis nucleoplasmin. At the amino acid level, O1-236 was 48% identical to Xenopus laevis nucleoplasmin (FIG. 4). Based on this homology and the expression patterns of both gene products in oocytes, the inventors termed the gene Npm2 since it was the mammalian ortholog of Xenopus laevis nucleoplasmin [called Xnpm2 in (MacArthur et al., 1997)]. Thus, herein after O1-236 is referred to as Npm2.

### Example 8

### Cloning of Human NPM2

Using the Npm2 cDNA sequence to search the EST database, two human cDNA clones containing sequences homologous to the mouse Npm2 were found. Sequence analysis of these two ESTs was performed. The two independent clones formed a 923 bp overlapping contig which encoded a 214 amino acid open reading frame. At the amino acid level, human NPM2 was 48% and 67% identical to Xnpm2 and mouse NPM2, respectably (FIG. 4).

Still further, FIG. 4 shows that the 207 amino acid of the mouse NPM2 shares 39.5% identity with Xenopus NPM2. Subsequently, human and rat NPM2 proteins were 61.4% and 81.6% identical with mouse NPM2.

When the frog and mammalian NPM2 sequences were compared, several interesting features were realized. Nucleoplasmin had a bipartite nuclear localization signal consisting of KR-(X)10-KKKK (Dingwall et al., 1987). Deletion of either of these basic amino acid clusters in nucleoplasmin prevented translocation to the nucleus (Robbins et al., 1991). When the mouse and human NPM2 sequences were analyzed, this bipartite sequence was 100% conserved between the two proteins (FIG. 4). Thus, mammalian NPM2 was predicated to translocate to the nucleus where it would primarily function.

Also, conserved between NPM2 and nucleoplasmin was a long stretch of negatively charged residues. Amino acids 125-144 of NPM2 and amino acids 128-146 of nucleoplasmin are mostly glutamic acid and aspartic acid residues, with 19 out of the 20 residues for NPM2 and 16 out of the 19 residues for nucleoplasmin either Asp or Glu. This region of Xenopus laevis nucleoplasmin has been implicated to bind the positively charged protamines and histones. Thus, a similar function for this acidic region of NPM2 was predicted.

The last obvious feature of the NPM2 and nucleoplasmin sequences was the high number of serine and threonine residues. The NPM2 sequence contained 19 serine and 17 threonines (i.e., 17.2% of the residues) and nucleoplasmin had 12 serine and 11 threonine residues (i.e., 11.5% of the residues). Several putative phosphorylation sequences that were conserved between the two proteins are shown in FIG. 4. Phosphorylation of nucleoplasmin is believed to increase its translocation to the nucleus and also its activity (Sealy et al., 1986, Cotten et al., 1986, Vancurova et al., 1995, Leno et al., 1996). Similarly, phosphorylation may also alter NPM2 activity. It is envisioned that phosphorylation may act to regulate when NPM2 acts, making it inactive until the critical time (i.e., histone addition to male and female pronuclei or during transcriptional arrest).

A specific putative phosphorylation site is, for example, casein kinase II. Casein kinase II specifically interacts with nucleoplasmin and phosphorylates it, and an inhibitor of casein kinase II blocks nuclear transport of Xenopus laevis nucleoplasmin (Vancurova et al., 1995). Interestingly, two of the predicted casein kinase II phosphorylation sites are conserved between frog nucleoplasmin 2 (Ser125 and Ser177), mouse NPM2 (Thr123 and Ser184), and human NPM2 (Thr127 and Ser191). Although other phosphorylation sites are likely important, a casein kinase II-NPM2 interaction in vivo may be predicted in mammals.

Since both mouse and human NPM2 and Xenopus laevis nucleoplasm are oocyte (and egg)-specific at the mRNA level and share highest identity, it was concluded that mammalian NPM2 and frog nucleoplasmin were orthologs.

### Example 9

### Structure of the Npm2 Gene

One of the full length Npm2 cDNAs (clone 236-1) was used to screen a mouse 129/SvEv genomic library (Stratagene) to identify the mouse Npm2 gene. 500,000 phage were screened and 12 positive were identified. Two of these overlapping phage clones, 236-13 and 236-14 (˜37 kb of total genomic sequence), were used to determine the structure of the mouse Npm2 gene. The mouse Npm2 was encoded by 9 exons and spanned ˜6.6 kb (SEQ ID NO: 7). Two moderate size introns (introns 4 and 5) contributed the majority of the gene size. The initiation ATG codon resided in exon 2 and the termination codon in exon 9. The splice donor and acceptor sites (SEQ ID NO: 7) matched well with the consensus sequences found in rodents, and all of the intron-exon boundaries conformed to the “GT-AG” rule (Senaphthy et al., 1990). A consensus polyadenylation signal sequence was found upstream of the polyA tracts which were present in the two isolated cDNAs (SEQ ID NO: 5).

### Example 10

### Chromosomal Mapping of the Mouse Npm2 Gene

Chromosomal mapping of genes in the mouse identifies candidate genes associated with spontaneous or induced mouse mutations. To further aid in the functional analysis of the isolated novel ovary-specific cDNAs, these mouse genes were mapped using the Research Genetics Radiation Hybrid Panel. Table 3 shows the genes that were mapped using this technique. Also, identification of the syntenic region on the human chromosome may identify one or more of these novel ovarian genes as candidate genes for known human diseases which map to these regions.

TABLE 3
Analysis of partial or full-length cDNAs

| PO1 cDNA | ORF | Database Homolog |
|---|---|---|
| O1-180 | 361 aa | No |
| O1-184 | 426 | No |
| O1-236 | 207 | Yes; Xenopus laevis |
| | | nucleoplasmin homolog |
| | | (81% similar) |

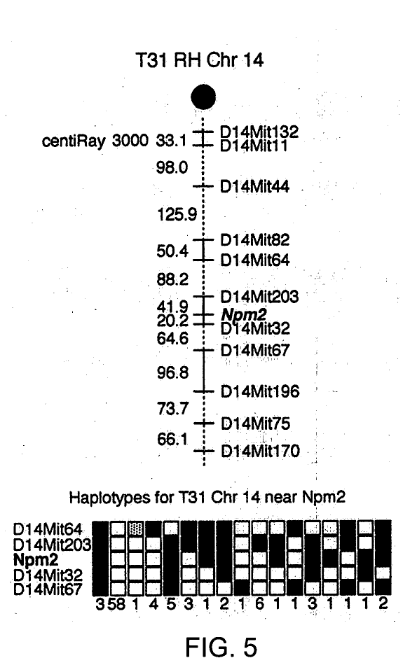

To map the mouse Npm2 gene, the inventors used the Research Genetics radiation hybrid panel, The Jackson Laboratory Backcross DNA Panel Mapping Resource, and The Jackson Laboratory Mouse Radiation Hybrid Database. Forward (SEQ.ID.NO.17: GCAAAGAAGCCAGTGACCAAGAAATGA) and reverse (SEQ.ID.NO.18: CCTGATCATGCAAATTTTATTGTGGCC) primers within the last exon were used to PCR amplify a 229 bp fragment from mouse, but not hamster. Using these primers, the mouse Npm2 gene was mapped to the middle of chromosome 14 (FIG. 5). Npm2 showed linkage to D14Mit32 with a LOD of 11.2 and also had a LOD of 7.8 to D14Mit203. This region was syntenic with human chromosome 8p21.

### Example 11

### Ovarian-Specific Expression of Mouse Npm2

In situ hybridization was performed as described previously (Albrecht et al., 1997; Elvin et al., 1999) and Example 4.

Briefly, ovaries were dissected from C57Bl6/129SvEv mice and fixed overnight in 4% paraformaldehyde in PBS before processing, embedded in paraffin and sectioned at 5 um. The fragment Npm2 was used as the template for generating sense and antisense strands with [α³²P]-dUTP using the Riboprobe T7/SP6 combination system (Promega). Hybridization was carried out at 50-55° C. with 5×10⁶ cpm for each riboprobe per slide for 16 hours in 50% deionized formamide/0.3 M NaCl/20 mM Tris-HCl (pH 8.0)/5 mM EDTA/10 mM NaPO₄ (pH8.0)/10% dextran sulphate/1× Denhardts/0.5 mg/ml yeast RNA. High stringency washes were carried out in 2×SSC/50% formamide and 0.1×SSC at 65° C. Dehydrated sections were dipped in NTB-2 emulsion (Eastman Kodak, Rochester, N.Y.) and exposed for 4-7 days at 40° C. After the slides were developed and fixed, they were stained with hematoxylin and mounted for photography.

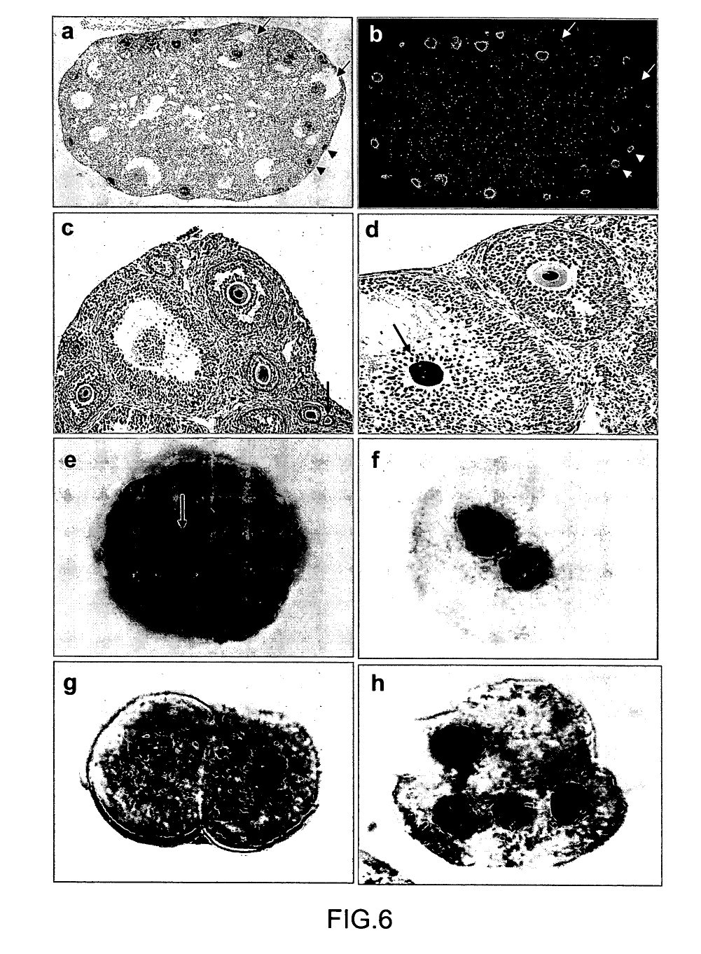

The Npm2 gene product was oocyte-specific (FIGS. 6A and 6B). The probe demonstrated specific expression in all growing oocytes. Oocyte-specific expression was first seen in the early one layer primary follicle (type 3a), with higher expression in the one layer type 3b follicle and all subsequent stages including antral (an) follicles. The “sense” probe did not detect a signal for this oocyte-specific gene.

### Example 12

### Subcellular Localization of NPM2

The subcellular localization of NPM2 protein was determined by immunohistostaining of mouse ovaries with anti-NPM2 antibodies.

The cDNA encoding the full-length mouse NPM2 protein was amplified by PCR to introduce a BamH1 site before the start codon and a XhoI site before the stop codon. This PCR fragment was cloned into pET-23b(+)(Novagen) to produce a His-tagged NPM2 protein and sequenced to confirm the absence of mutations. The recombinant NPM2 protein was purified as described in the pET System Manual (Novagen). Two goats were immunized with the purified His-tagged NPM2 to produce specific and high affinity antibodies.

Ovaries were fixed in 4% paraformaldehyde in PBS for 2 h, processed, embedded in paraffin, and sectioned at 5 um thickness. Goat anti-NPM2 polyclonal antiserum was diluted 1:2000 in Common Antibody Dilute (BioGenex). The pre-immune goat serum from the same goat was used as a control. All sections were blocked for 10 min in Universal Blocking Reagent (BioGenex), and incubated with the primary antibody for 1 h at room temperature. NPM2 detection was accomplished using anti-goat biotinylated secondary antibody, streptavidin-conjugated alkaline phosphatase label and New Fuschin substrate (BioGenex Laboratories, Inc., San Ramon, Calif.).

One to eight-cell embryos and blastocysts were fixed in 4% paraformaldehyde in PBS for 2 h in 96-well round bottom plate, washed with 0.85% saline, and embedded in a few drops of 1.5% agarose. The agarose-containing embryos were dehydrated, embedded in paraffin, and analyzed as described above.

Consistent with the expression pattern of Npm2 mRNA, NPM2 protein was expressed in oocytes from type 3 to antral follicle stages. In randomly cycling mice, the anti-NPM2 antibody strongly and specifically stained the nucleus (FIG. 6C). The oocyte nucleus is also called the germinal vesicle (GV). The preovulatory surge of luteinizing hormone (LH) accelerates the maturation of GV oocytes and promotes GV breakdown (GVB). When mice were injected with PMSG and hCG to induce superovulation, the NPM2 protein redistributed in the oocytes of antral follicles after germinal vesicle breakdown. In preovulatory GVB oocytes, the NPM2 was evenly distributed in the cytoplasm of the oocyte (FIG. 6D). Since xNPM2 has been implied to play a role in sperm DNA decondensation and pronuclei formation after fertilization, this redistribution suggested that the cytoplasmic NPM2 was now properly positioned to interact with the sperm nucleus at the time of fertilization. To examine the NPM2 expression after fertilization, early embryos were fixed, sectioned and stained with anti-NPM2 antibodies. In zygotes, NPM2 began to translocate back to the nucleus. FIG. 6E shows an intermediate stage in which one pronucleus was formed but other was not yet complete and some NPM2 was still present in the cytoplasm. At a later point (FIG. 6F), all of the NPM2 was present in the pronuclei. In two-cell (FIG. 6G) and eight-cell (FIG. 6H) embryos, the antibody continued to detect the NPM2 protein exclusively in the nucleus. NPM2 continued to be detected at significantly reduced levels in blastocysts (embryonic day 3.5), but in embryonic day 6.5 embryos, NPM2 expression was undetectable.

### Example 13

### Generation of Npm2 Knockout Mice

To study the role of NPM2 in mammalian oocyte development and early embryo development, the inventors disrupted the mouse Npm2 locus using ES cell technology.

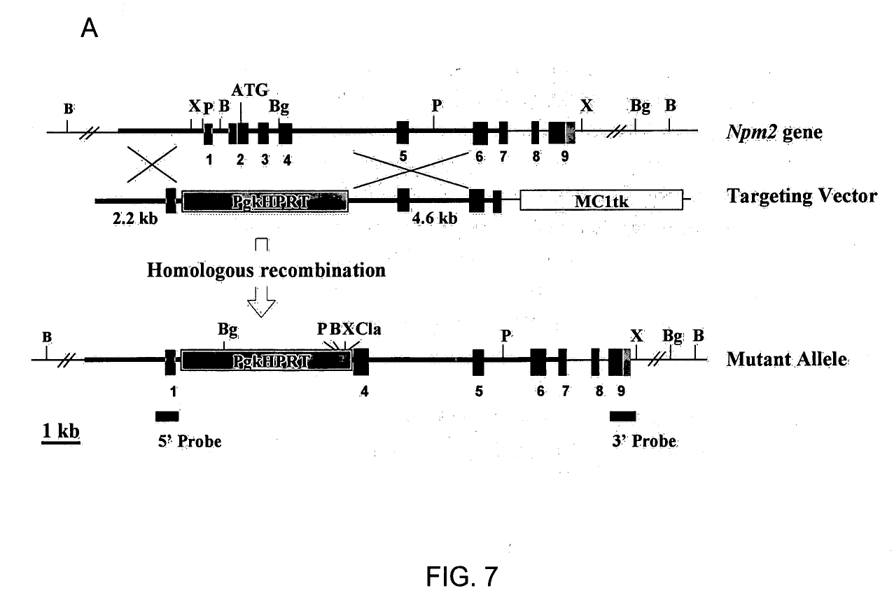

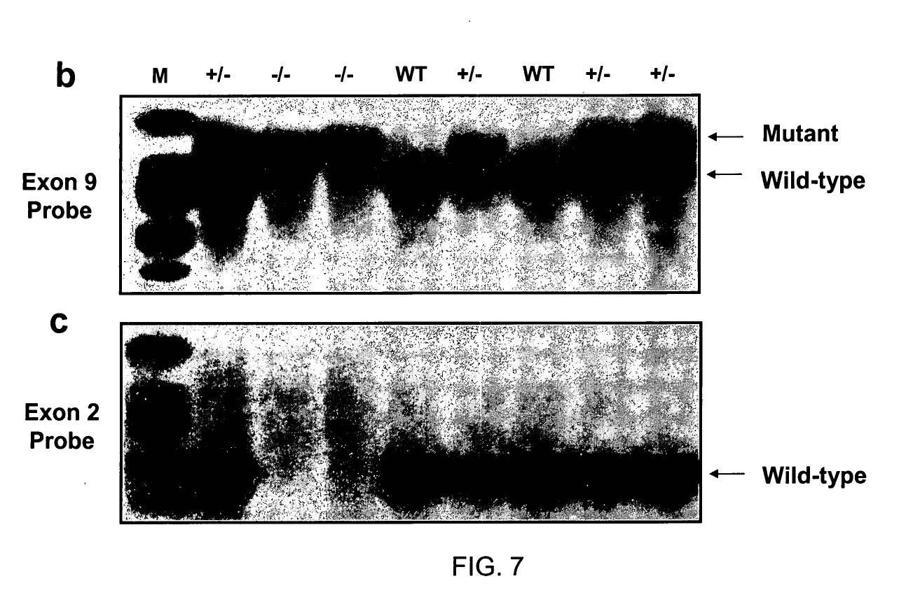

The targeting vector was constructed to delete exon 2 which contains the translation initiation codon and also exon 3 and the exon 4 splice junction (FIG. 7A). Outside of exon 2, only one other ATG was present in the remaining sequence (exon 6), and this ATG was positioned downstream of the acidic domain and between the bipartite nuclear localization consensus sequence. The deletion targeting vector contains from left to right, 2.2 kb of 5′ Npm2 homology, a PGK-hprt expression cassette, 4.6 kb of 3′Npm2 homology and an MC1-tk (thymidine kinase) expression cassette. The linearized Npm2 targeting vector was electroporated into AB2.1 ES cells. ES cell clones were selected in M15 medium containing HAT (hypoxanthine, aminopterine and thymidine and FIAU [1-(2′-deoxy-2′-fluoro-B-D-arabinofuranosyl)-5′-iodouracil]. Culturing of ES cells and collection and injection of blastocysts (Matzuk et al., 1992).

For genomic Southern blot analysis, BglII-digested DNA was transferred to GeneScreen Plus nylon membrane and probed with an external 190 bp PCR synthesized fragment corresponding to exon 9 sequence (3′ probe). An internal 200 bp PCR synthesized fragment (49 bp exon 1 plus 150 bp 5′ upstream sequence) was also used to distinguish the wild-type and Npm2 null (Npm2^{tm1Zuk}, herein called Npm2^{-/-}) alleles when DNA was digested with BamH1. Genotype analysis of 230 F2 offspring from these intercrosses (FIG. 7B; Table 4) was consistent with a normal Mendelian ratio of 1:2:1, and a similar number of male and female homozygotes (Npm2^{-/-}) were produced. Therefore, Npm2 homozygous mutant male and female mice were viable and appeared to have normal sexual differentiation demonstrating that Npm2 was not required prior to birth.

TABLE 4
Heterozygous mating

| | −/− | +/− | Wild type | Total |
|---|---|---|---|---|
| Male | 27 | 71 | 19 | 117 |
| Female | 27 | 53 | 33 | 113 |
| Total | 54 | 124 | 52 | 230 |
| % | 23 | 54 | 23 | 100 |

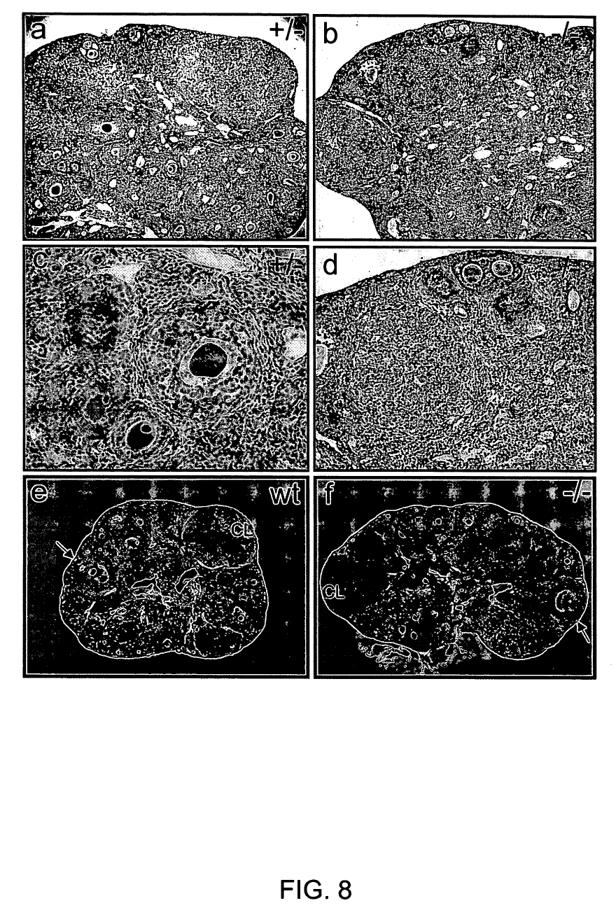

To confirm that the mice genotyped as Npm2 homozygotes lacked Npm2, a cDNA probe that hybridized to exon 2 of the wild-type Npm2 gene was used for Southern blot analysis. As shown (FIG. 7C), this probe failed to detect any signal in DNA derived from homozygous (Npm2^{-/-}) mice in which exon 2 had been deleted. Furthermore, Npm2 immunohistochemical analysis was performed on Npm2 homozygotes and controls. Whereas the expression of NPM2 protein was noted in the ovaries from the heterozygous controls (FIGS. 8A and 8C), no protein was detected in oocytes in the homozygote ovaries (FIGS. 8B and 8D).

This confirmed that the Npm2^{tm1Zuk} mutation was a null allele and that Npm2 homozygotes were completely lacking NPM2 protein.

### Example 14

### Loss of NPM2 Results in Female Infertility and Subfertility

To study the function of NPM2 in reproductive function, adult homozygous hybrid (C57Bl/6/129SvEv) male or female mice were intercrossed with control hybrid mice (C57Bl/6/129SvEv) mice. Consistent with the female-specific expression of Npm2 mRNA and protein, Npm2^{-/-} male mice were fertile and had no gross or histological defects in the testes. Similarly, intercrosses of 9 female and male Npm2^{+/-} males over 6 months resulted in a normal number of litters (n=54; 1.00±0.06 litters/month), with 8.98±0.31 offspring/litter. In contrast, only 11 of 14 Npm2^{-/-} females became pregnant over this period, yielding 40 litters (0.48±0.11 litters/month) with 2.65±0.24 offspring/litter. Thus, deficiency of NPM2 leads to subfertility and infertility in females, but not males.

### Example 15

### Early Cleavage Defect in Npm2-Null Fertilized Eggs

To determine the causes of the fertility defects in the Npm2^{-/-} female mice, ovaries were first examined morphologically and histologically. There was no significant difference between Npm2^{-/-} and control ovaries at the gross or histological levels (FIGS. 8E and 8F). Normal folliculogenesis including the formation of corpora lutea were observed in the Npm2^{-/-} ovaries suggesting that ovulation occurred in these mice.

To confirm that ovulation was occurring and to further study the cause of the infertility and subfertility of the Npm2^{-/-} mice, pharmacological superovulation of wild-type, heterozygous, and homozygous mice was performed and the eggs were collected from the oviducts and cultured in vitro as described in Example 5.

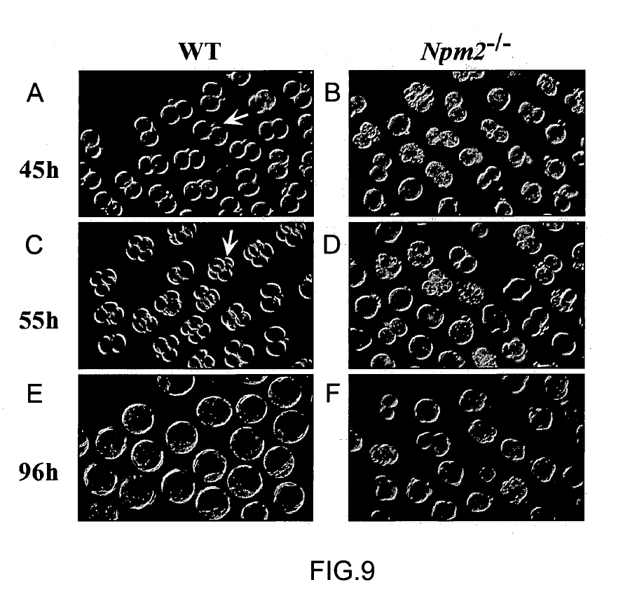

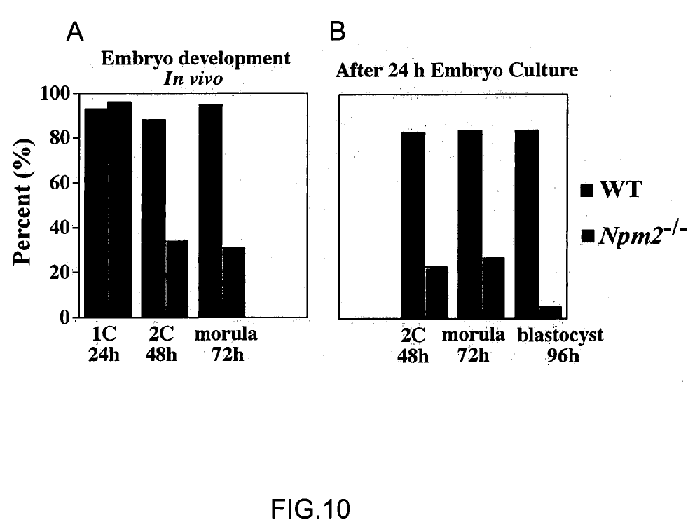

In vitro maturation and fertilization of Npm2 null eggs were apparently normal, but there was reduced cleavage to the 2-cell stage (Table 5). In vivo fertilized Npm2 null eggs were recovered 24 hours after hCG treatment. However, mostly asynchronously fragmenting and dying embryos were found 45-55 hours post-hCG (FIG. 10A, FIGS. 9A-9D), and few Npm2 null embryos were cultured to the blastocyst stage (FIG. 10B, FIGS. 9G-9H). Thus, the defect in Npm2 null mice appeared to result in a reduced viability of embryos.

TABLE 5
Early developmental potential of eggs matured and fertilized in vitro

| | Number | | | | |
|---|---|---|---|---|---|
| | of | Oocytes | Eggs | Eggs | 2-cell stage |
| Genotype | females | recovered | matured | fertilized | embryos (%)* |
| Wild type | 4 | 93 | 69 | 48 | 46 (96%) |
| Npm2^{-/-} | 4 | 75 | 62 | 49 | 9 (18%)** |

*Percentage of eggs fertilized proceeding to 2-cell stage embryos.
**P < 0.0005 (χ² test)

### Example 16

### DNA Damage

Next, TUNEL (TdT-mediated dUTP nick end labeling) assays were performed to determine DNA damage. TUNEL assays rely on a terminal deoxynucleotidyl transferase (TdT) to label free ends of DNA with fluorescent dUTP conjugates.

Briefly, oocytes and early embryos were collected from oviducts, fixed, permeabilized, and incubated with TdT and labeled nucleotides. These were then washed and imaged by deconvolution microscopy. For BrDU incorporation assays, fully-grown oocytes were in vitro matured and fertilized as described above. Approximately 8 h after fertilization, zygotes that had formed pronuclei were transferred to medium supplemented with 50 μM BrDU for overnight culture (Ferreira et al., 1997). Incorporation was assessed by immunofluorescence using a mouse monoclonal antibody against BrDU (Roche, #1170376).

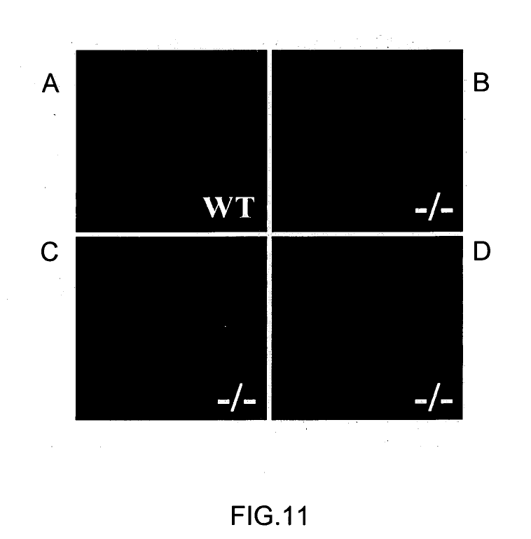

Nuclei of these embryos were TUNEL positive, although there was no evidence that DNA damage caused embryo loss, and 1-cell embryos collected from null females 20 hrs post-hCG exhibited TUNEL staining only within polar bodies (FIGS. 11A-11D). All developmental defects occurred when eggs were fertilized with wild-type spermatozoa, indicating that maternal NPM2 was crucial in early embryogenesis.

### Example 17

### Transcription-Requiring Complex (TRC) Quantification

TRC proteins were extracted as described by Conover et al., 1991. Briefly, two-cell embryos estimated to be in early S-phase were collected from oviducts and cultured for two hours in M16 media supplemented with amino acids, including ³⁵S-methionine. The addition of 1 μg/mL actinomycin D (Sigma #A1410) served as a negative control. Insoluble proteins remained in the zona after extraction with 2% Triton X-100, 0.3 M KCl, and 50 mM Tris-HCl pH 7.4. These proteins were electrophoresed, and the gel was then fixed in isopropanol and glacial acetic acid, soaked in Amplify (Amersham Pharmacia Biotech), and exposed to X-OMAT film or phosphorimaged overnight.

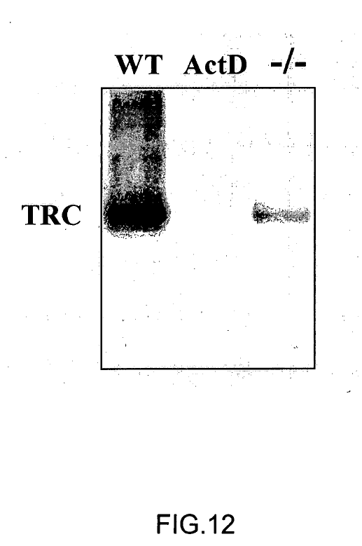

Two-cell embryos lacking NPM2 synthesized the Transcription-Requiring Complex (TRC) of proteins, which indicated some zygotic gene transcription and translation (Latham et al., 1992), albeit at reduced levels (30%) as compared to wild-type 2-cell embryos (FIG. 12). It has been suggested that NPM2 may function in the translational activation of specific maternal maternal RNAs in early embryos as has been proposed for xNPM2 (Meric et al., 1997). Because a few Npm2 null embryos developed to birth, potential compensatory mechanisms exist. Transcriptional activation of paternal Npm2 was not involved as Npm2^{-/-} males sire pups when mated with Npm2^{-/-} females.

### Example 18

### Analysis of WT and Mutant Oocytes and Embryos

Immunofluorescence of formaldehyde-fixed unfertilized eggs and early embryos was undertaken as described previously (Yan et al., 2002) to analyze WT and mutant oocytes and embryos.

Briefly, oocytes were collected and fixed in 2-4% formaldehyde or 70% ethanol, blocked in PBS with 10% fetal calf serum, permeabilized with Triton X-100, and treated with primary and secondary antibodies. After washing, DNA was counterstained with DAPI or To-pro-3 and images were taken using confocal or deconvolution microscopy. The following primary antibodies were used: goat NPM2 antisera (1:500); rabbit anti-acetyl-Histone H3 (Upstate Biotechnology 06-599; 1:200); goat anti-fibrillarin (Santa Cruz Biotechnology sc-11335; 1:100); mouse monoclonal anti-tubulin antibody (Sigma T-6793; 1:300); goat anti-lamin B (Santa Cruz sc-6217; 1:300); mouse anti-hypoacetylated histone H3 (Upstate Biotechnology 06-755; 1:500); rabbit anti-histone H3 phosphorylated at Ser10 (Upstate Biotechnology 06-570; 1:500); and mouse monoclonal anti-histone H1 (Santa Cruz sc-8030; 1/200). The following secondary antibodies were used: AlexaFluor S94 rabbit anti-goat (Molecular probes A-11080; 1:500); AlexaFluor568 goat anti-rabbit (Molecular Probes A-11011; 1:500); and AlexaFluor488 goat anti-mouse (Molecular Probes A-11001; 1:500).

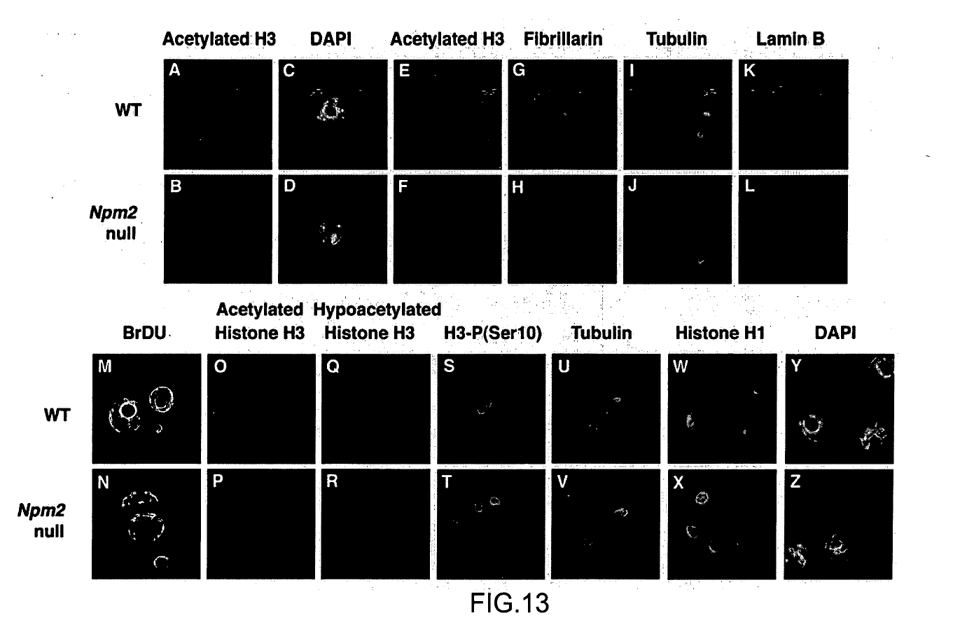

Oocytes from PMSG-treated wild-type females exhibited an organization of heterochromatin surrounding the prominent nucleolus, termed the SN (surrounded nucleolus) configuration (FIG. 13C). The SN configuration was characteristic of advanced oocyte development, as SN oocytes were larger and were found in gonadotropin-dependent follicles. The condensation of chromatin correlated with transcriptional silencing, competence to resume meiosis, the appearance of M-phase characteristics, and post-fertilization embryo developmental potential (Bouniol-Baly et al., 1999; Mattson et al., 1990; Wickramasinghe et al., 1991; Zuccotti et al., 1998). In contrast to wild-type oocytes, the DNA in Npm2 null oocytes was amorphous and diffused with no condensation around the nucleolus (FIG. 13D). The loss of nucleolar clearing was also illustrated by immunofluorescence to detect acetylated histone H3 in these oocytes (FIGS. 13E-13F), as well as the less mature non-SN oocytes isolated from 10 day old untreated mice (FIGS. 13A-13B). Immunofluorescence to localize the nucleolar protein fibrillarin demonstrated dispersed nucleolar-like bodies in Npm2 null oocytes compared to the single organized nucleolus observed in controls (FIGS. 13G-13H). These anomalies were observed in hundreds of oocytes examined from more than 30 Npm2^{-/-} females. Thus, NPM2 was essential for organization of oocyte nuclear and nucleolar domains and the compaction of oocyte chromatin during the final stages of oocyte development.

Meiosis progresses essentially normally in the absence of NPM2, with no obvious defects in metaphase II arrest, spindle formation, chromosomal segregation, or extrusion of polar bodies (FIGS. 13I-13J). Sperm DNA decondensation occurs normally without NPM2; fertilization was followed by the formation of both maternal and paternal pronuclei surrounded by nuclear envelopes (FIGS. 13K-13L), and there was no persistent protamine 2B detectable in male pronuclei (data not shown). DNA replication in the first S phase also proceeded without NPM2 (FIGS. 13M-13N). However, as in Npm2^{-/-} oocytes, normal nucleoli were absent in Npm2 null 1-cell embryos, and immunofluorescence to detect acetylated histone H3 in zygotes showed no nucleolar clearing compared to controls (FIGS. 13O-13P). Hypoacetylated histone H3, which was normally associated with compact chromatin rimming the pronuclei nucleoli, was undetectable (FIGS. 13Q-13R). Treatment with colcemid to inhibit spindle formation arrested both wild-type (n=34) and Npm2 null (n=28) 1-cell embryos in metaphase with condensed chromosomes staining for phosphorylated histone H3; this indicated that the first mitosis initiated in essentially all cases (FIGS. 13S-13T). Without colcemid treatment, spindle forms in wild-type zygotes 13-15 hours after pronuclear formation (FIG. 13U), and all zygotes complete mitosis by 19 hours. In contrast, Npm2 null zygotes were observed with metaphase spindle from 13-19 hours following pronuclear formation (FIG. 13V) and immediately preceding fragmentation, suggesting abnormal exit from the first mitosis. A few multi-cellular embryos lacking NPM2 were recovered, and their nuclei contain somatic linker histone H1 (FIGS. 13W-13X); however, their nuclei remained relatively amorphous until the blastocyst stage (FIGS. 13Y-13Z). Thus, mammalian NPM2 was crucial for histone deacetylation and heterochromatin formation surrounding nucleoli in oocytes and early zygotes.

### Example 19

### RNAse Protection Assay

An RNAse protection assay was performed to quantify 18S and 28S rRNAs in wild-type (WT) and Npm2 null GV stage oocytes, metaphase II oocytes, and 1-cell embryos. ³²P-labeled antisense probes for 18S and 28S rRNAs were prepared using the Ambion MAXIscript kit templates (Austin, Tex.). Total RNA from 30 oocytes or embryos was prepared for probe hybridization using the Ambion Direct Protect Lysate kit and then incubated with probe, treated with nuclease cocktail, and electrophoresed as recommended by the manufacturer and as described in (Tong et al., 1995). Protected fragments were detected by autoradiography and quantified by phosphorimaging (Johnston et al., 1990).

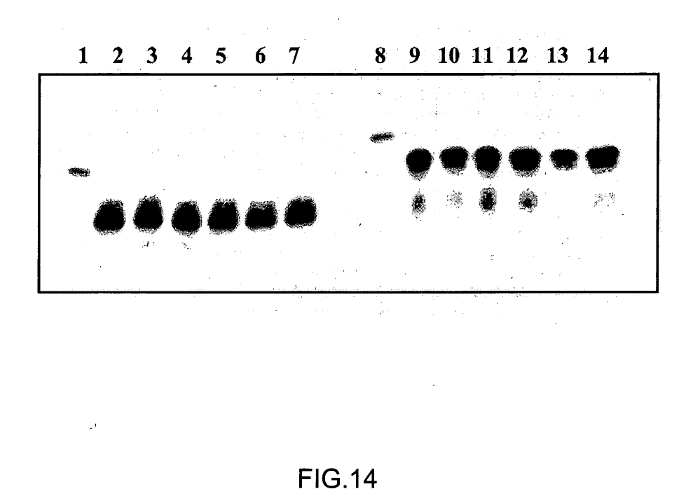

As shown in FIG. 14, there are no major differences apparent in ribosomal RNA content in the absence of NPM2.

### Example 20

### Total Protein Synthesis

Absolute rates of protein synthesis were quantified as described by Schultz et al., 1978. Briefly, GV-stage oocytes, metaphase II oocytes, and 1-cell embryos were collected and incubated for 2 hours in M16 media supplemented with amino acids, including 250 μCi of ³⁵S-methionine/mL, and either 0.3 mg/mL or 3.0 mg/mL of non-radioactive methionine. After the incubation, twenty oocytes or embryos were removed from each group; extensively washed in fresh M16 media, and lysed by freezing and thawing. Total protein was precipitated by the addition of 20 μL of 1 ug/uL BSA and 20 μL of 10% trichloroacetic acid (TCA). Pellets were washed with 5% TCA, dissolved in 1M NaOH for 1 hour at 37° C., acidified with HCl, and assayed by scintillation counting.

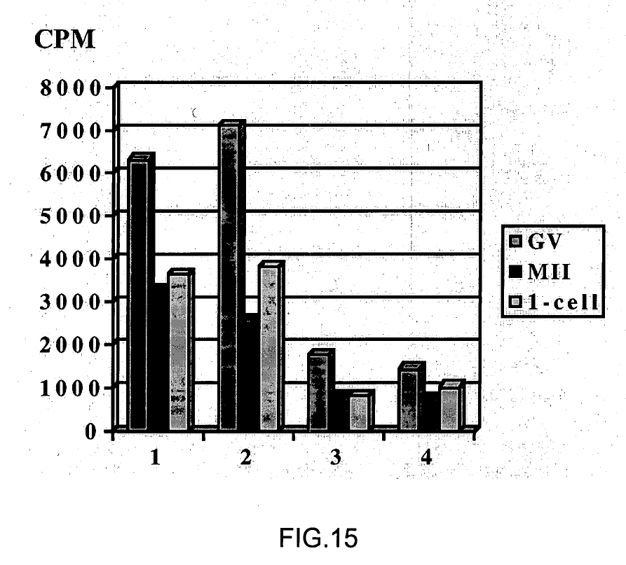

As shown in FIG. 15, WT and Npm2 null oocytes and embryos displayed comparable counts or levels of protein synthesis.

### Example 21

### In Situ Hybridization to Detect Npm1 and Npm3

Despite the low homology in the primary amino acid sequences of NPM2 and other “ubiquitous” nucleoplasmins (Chan et al., 1989; MacArthur et al. 1997: Schmidt-Zachmann et al., 1988), these more widely expressed nucleoplasmins are karyophilic, negatively charged proteins that may share functional redundancy with NPM2 in oocytes and developing embryos.

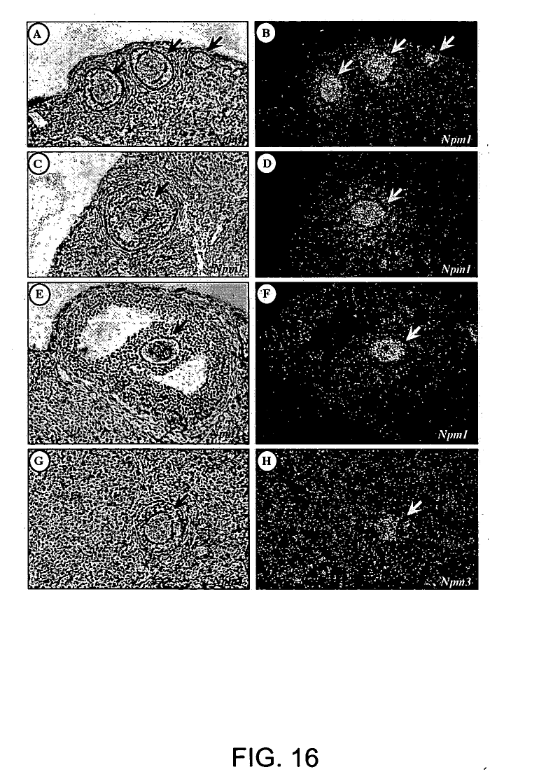

In situ hybridization to measure Npm1 and Nmp3 mRNA in mouse oocytes was performed as described in Example 4. Npm1 mRNA was detected with an ³⁵ S-UTP labeled antisense riboprobe corresponding to nucleotides 131-551 of NM_{—}008722. FIGS. 16A-16F shows that Npm1 mRNA was highly expressed in oocytes of small follicles (A-B), secondary follicles (C-D) and large antral follicles (E-F) (arrows). Sections are shown in brightfield (A, C, and E) and darkfield (B, D, and F) to demonstrate the histology and highlight the hybridization signal, respectively. Npm3 mRNA was studied using a probe corresponding to 41-657 of NM_{—}008723. FIG. 16G-FIG. 16H show that Npm3 mRNA was detected in all stages of oocytes in the adult ovary, although at levels more comparable to the expression observed in the surrounding somatic cells (G-H). Thus, in situ hybridization revealed that both nucleophosmin 1 (Npm1; B23) and nucleoplasmin 3 (Npm3) mRNAs were expressed in mouse oocytes.

### Example 22

### Cloning of Zar1

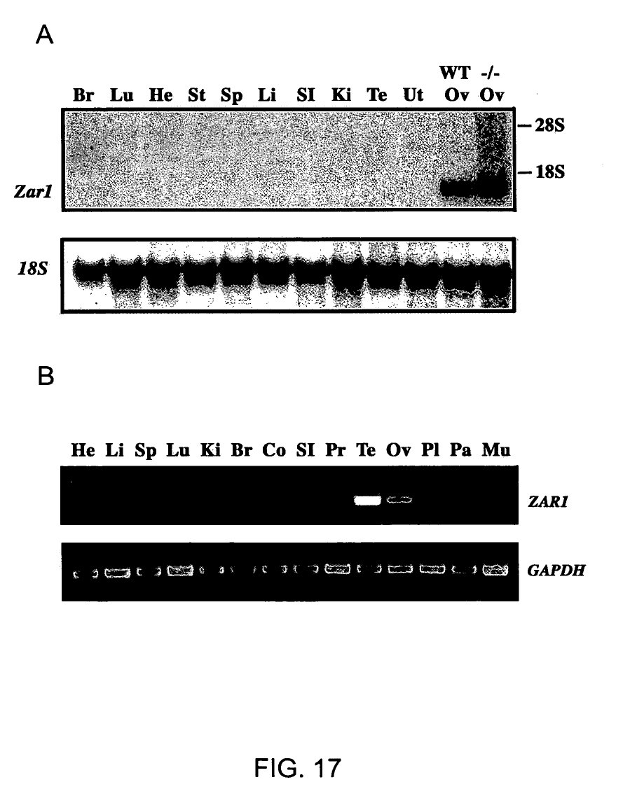

Partial cDNAs from the library of Example 1 were subcloned and sequenced and all sequences were grouped into contigs and analyzed by BLAST searches. Novel sequences were analyzed further by Northern blot analysis. A partial 325 nucleotide cDNA designated ovary 1-clone 180 [O1-180, herein after referred to as zygote arrest 1 (Zar1)] identified a 1.4 kb transcript only in the ovary (FIG. 17A).

Next, a ZAP-express mouse ovary cDNA library was screened to isolate the full-length Zar1 cDNA. Excluding the polyA tail, the full-length Zar1 cDNA was about 1.4 kb, and encoded an open reading frame from nucleotides 28 to 1110. The Zar1 cDNA was homologous to several ESTs in the database, including ESTs in a mouse sixteen-cell embryo cDNA library (AU044294) and a mouse unfertilized egg cDNA library (AU023153). The polypeptide predicted from the Zar1 cDNA ORF consisted of 361 amino acids (FIG. 11), with a molecular mass of 40 kDa. Searching the public protein database failed to identify any known protein homologues. A bipartite nuclear localization signal was found at positions 333 to 350 (SEQ.ID.NO.19: Lys-Arg-Pro-His-Arg-Gln-Asp-Leu-Cys-Gly-Arg-Cys-Lys-Asp-Lys-Arg-Leu-Ser), which strongly suggested that Zar1 migrates to the oocyte or embryo nucleus.

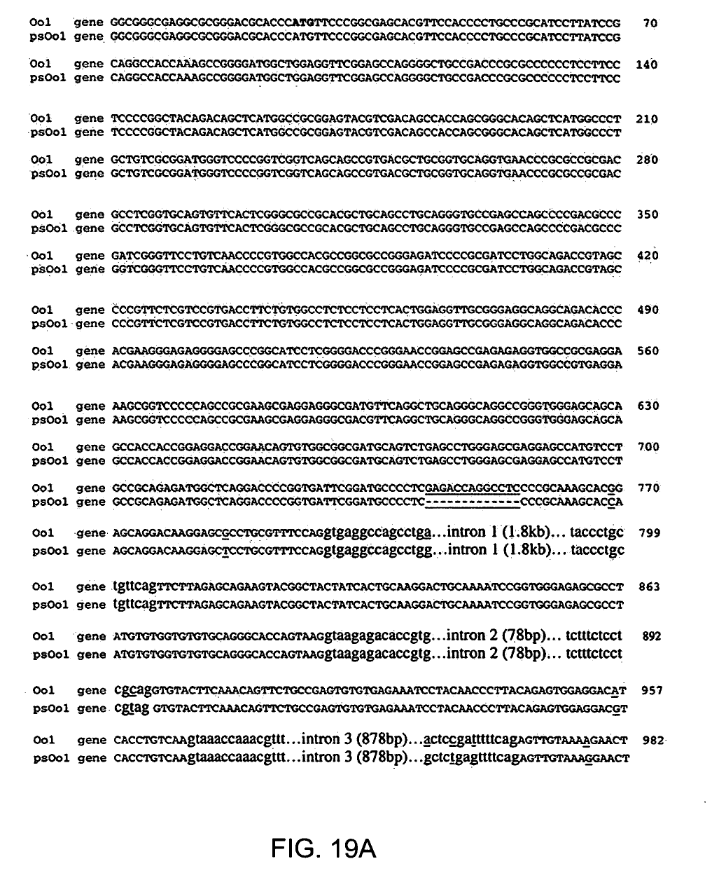

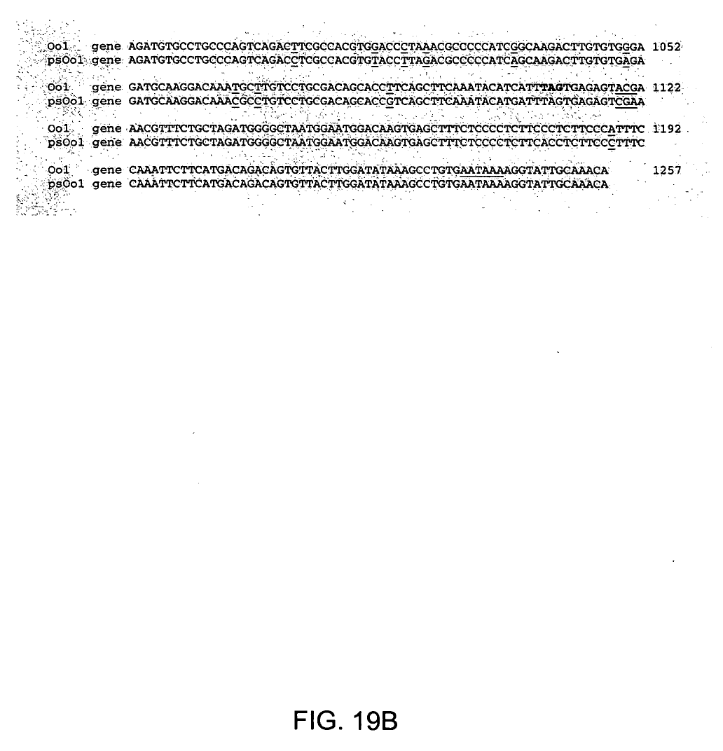

To clone the mouse Zar1 gene, both mouse 129/S6SvEv genomic λ Fix II phage and 129X1/SvJ BAC libraries were screened with the full-length Zar1 cDNA or PCR primers (SEQ.ID.NO.20 5′-CTAGAAAAGGGGACTGTAGTCACT-3′, and SEQ ID NO: 21 5′-TGCATCTCCCACACAAGTCTTGCC-3) and the recovered clones were characterized by Southern blot analysis and sequencing. The mouse Zar1 gene (SEQ ID NO:11) spanned 4.0 kb, and exon 1 encoded the majority of the protein. Both the Zar1 gene and a related pseudogene (Zar1-ps1) contained four exons. The related Zar1-ps1 (SEQ ID NO:12) gene contained a 13-nt gap in exon 1 (FIG. 19), which was predicted to result in a frameshift and early protein termination in exon 2. Whereas RT-PCR with Zar1-specific primers confirmed that it was ovary-specific, the related gene-specific primers failed to detect a transcript in all tissues examined. This established the related gene as a pseudogene (Zar1-ps1).

### Example 23

### Chromosomal Mapping of the Zar-1

The whole genome-radiation hybrid panel T31 (McCarthy et al., 1997) were purchased from Research Genetics (Huntsville, Ala.) and used according to the manufacturer's instruction. The panel was constructed by fusing irradiated mouse embryo primary cells (129aa) with hamster cells. Because the sequence of the hamster homologues for Zar-1 is unknown, the inventors designed the reverse primers from the 3′-untranslated region of the murine sequence to minimize the risk of coamplification of the hamster homologues (Makalowski and Boguski, 1998). Zar-1 gene specific primers were (SEQ.ID.NO.20) 5′-CTAGAAAAGGGGACTGTAGTCACT-3′ and (SEQ.ID.NO.21) 5′-TGCATCTCCCACACAAGTCTTGCC-3′; Zar-1-ps-1 gene specific primers were (SEQ.ID.NO.22) 5′-CTAGAAAAGGGGACTATAGGCACC-3′ and (SEQ.ID.NO.23) 5′-TGCATCTCTCACACAAGTGTTGCT-3′. Specificity of the two sets of primers was tested with A23 hamster DNA and 129 mouse DNA. The PCR reactions were performed in 15 μl final volume, containing 1 μl of each panel DNA, 1.25u of Taq platinum DNA polymerase (Gibco, Rockville, Md.), companion reagents (0.25 mM dNTPs, 1.5 mM MgCl₂, 1×PCR buffer), and 0.4 μM of each primer. An initial denaturation step of 4 min at 94° C. was followed by amplification for 30 cycles (40 s at 94° C., 30 s at 60° C., and 30 s at 72° C.) and final elongation at 72° C. for 7 min.

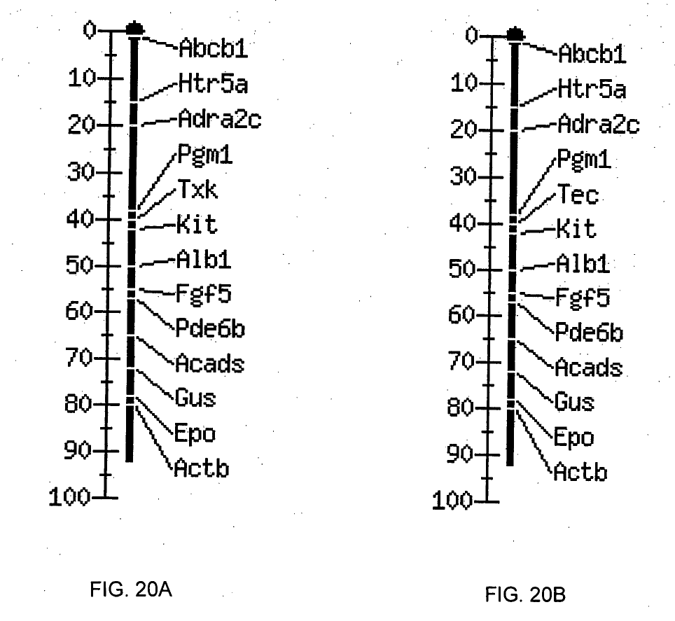

The data for each gene were submitted for analysis at the Jackson Laboratory Mouse Radiation Hybrid Mapper Server. Both genes were placed in the same region on mouse chromosome 5. The Zar-1 locus was at 40cM, between two markers D5Buc48 and Txk, while the Zar-1-ps1 gene lies at 41cM, between Tec and D5Mit356, just distal to the coding locus (FIG. 20).

### Example 24

### Isolation of Human ZAR1

To identify the human ortholog of the mouse Zar1 gene, a full-length mouse ovary cDNA was used for BLAST searches and to screen a human genomic library. A human genomic sequence was identified from both the non-redundant database and a human genomic library. The entire human gene spanned 4.1 kb and also contained four exons; its four exons shared 50%, 86%, 84%, and 78% nucleotide homology with mouse Zar1 exons 1 to 4, respectively. The ZAR1 gene was located on human chromosome 4p12, which is syntenic to the Zar1 locus on mouse chromosome 5. No pseudogene was found in the human genome.

### Example 25

### Expression of Human ZAR1

RT-PCR analysis of human ZAR1 was performed using standard techniques well known and used in the art. The following primers were used to amplify cDNA derived from multiple human tissues (SEQ.ID.NO.24) 5′-GGAGGTGTGGACGAAGAAGG-3′ and (SEQ.ID.NO.25) 5′-AAGCTGAAGGTGCTGTCGCAGG-3′. GAPDH was used as a control using the primers (SEQ.ID.NO.26) 5′-TGAAGGTCGGAGTCAACGGATTTGGT-3′, and (SEQ.ID.NO.27) 5′-CATGTGGGCCATGAGGTCCACCAC-3′.

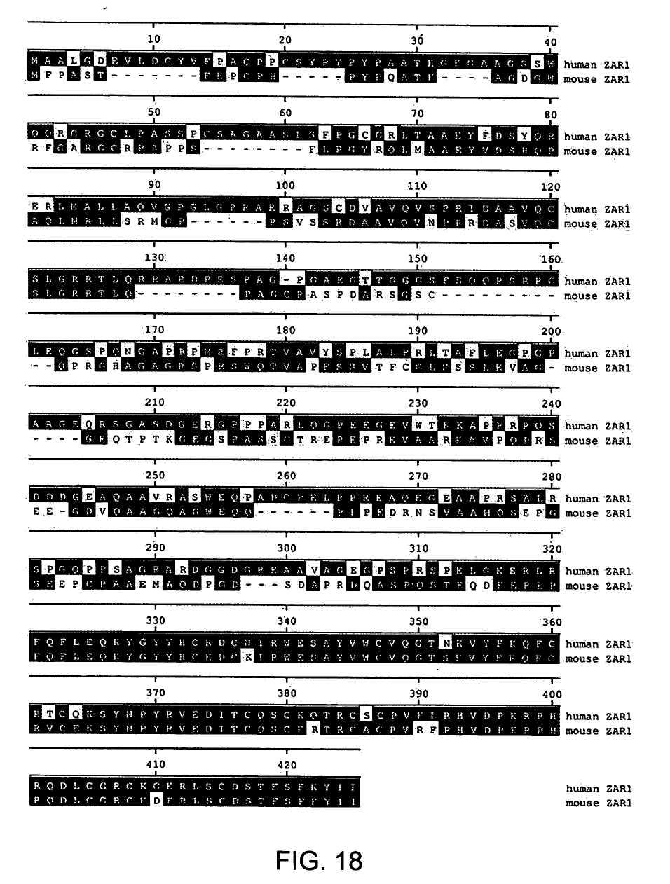

As shown in FIG. 17B, Human ZAR1 exons 1 to 4 were transcribed exclusively in the ovary and testis based on multiple-tissue RT-PCR analysis. The human ZAR1 mRNA is predicted to be at least 1.3 kb and encode a larger protein of 424 amino acids. Human and mouse ZAR1 proteins share 58% amino acid identity (FIG. 18) although the carboxyl-terminus of the ZAR1 proteins, encoded by exons 2-4, were highly conserved and showed 91% similarity between mouse and human. This suggests that the ZAR1 carboxyl-terminus region may be functionally more important.

### Example 26

### Protein Expression of ZAR1

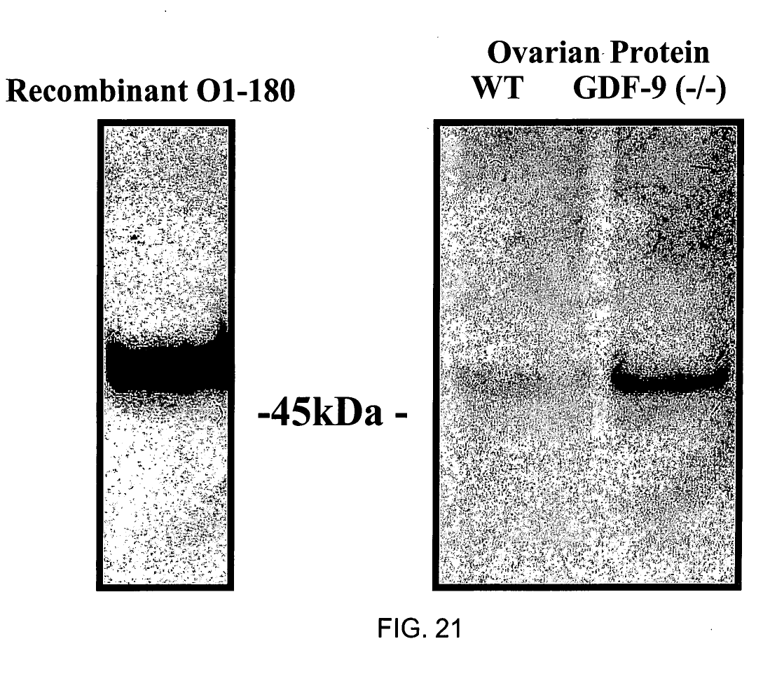

Western blot analysis was performed using standard techniques well known and used in the art. Briefly, ovarian protein was isolated from wildtype and Gdf9^{-/-} mice. Antibodies to ZAR1 were used to compare the size of the recombinant ZAR1 protein to a native ZAR1 protein. FIG. 21 revealed that the recombinant ZAR1 protein is similar in size to the native ZAR1 protein from isolated ovaries from Gdf9^{-/-} mice.

### Example 27

### Localization of Zar1 in Mouse Ovaries

In situ hybridization was performed with the Zar1 specific probe. [α-³⁵S]UTP-labeled antisense and sense probes were generated by the Riboprobe T7/T3 combination system (Promega, Madison, Wis.). Hybridization was carried out according to methods described by Albrecht et al., 1997 and Elvin et al., 1999A.

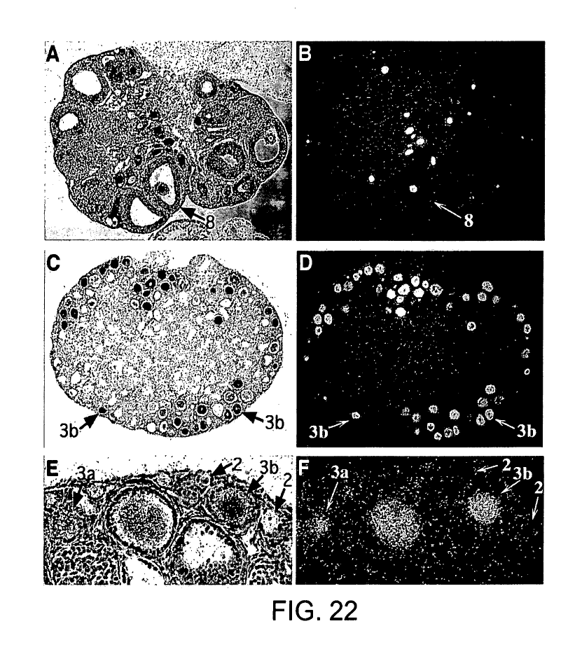

In situ hybridization showed high level expression of Zar1 localized to the oocytes within these ovaries. The expression of Zar1 within oocytes was evident at the one-layer (primary) follicle stage through the antral follicle stage, but no expression was observed at the primordial follicle stage. Because the number of follicles was increased in Gdf9 knockout ovaries due to the arrest of follicle development at the primary follicle stage, more Zar1 positive oocytes were detected in each section (FIG. 22).

### Example 28

### Generation of Zar1 Knockout Mice

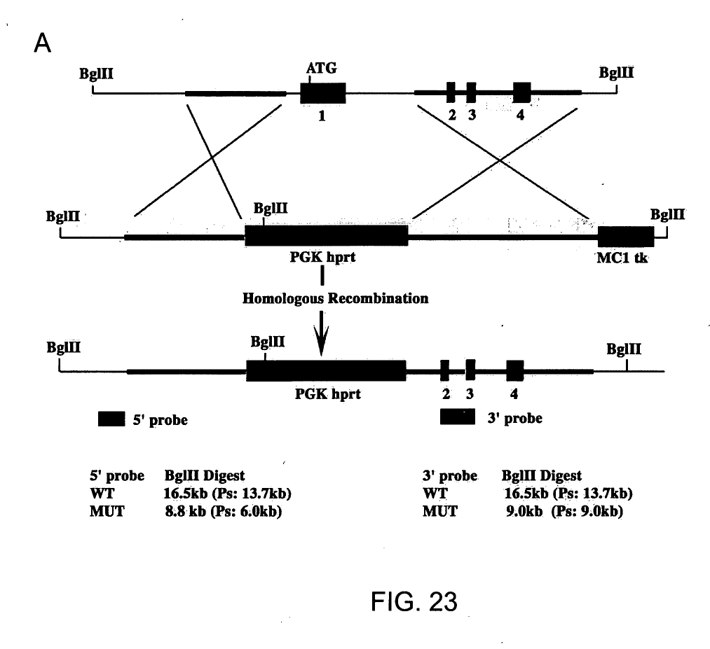

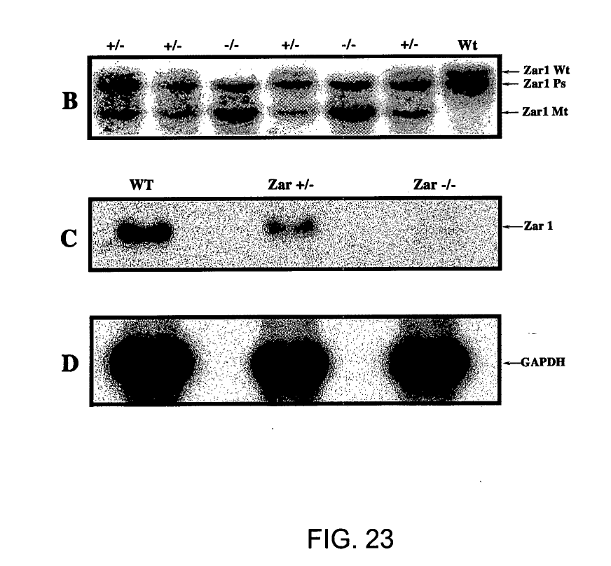

Mouse Zar1 genomic sequences were used to generate a targeting vector to mutate the Zar1 gene in ES cells. The targeting vector contained 1.5 kb of genomic DNA upstream of Zar1 exon 1, a selectable marker (the PGKhprt expression cassette), 5.5 kb of Zar1 sequence downstream of exon 1, and a negative selectable marker (the MC1tk expression cassette) (FIG. 23A). The linearized vector was electroporated into the hprt-negative AB2.2 ES cell line, clones were selected in HAT (hypoxantine, aminopteridine and thimidine) and FIAU [1-(2′-deoxy-2′fluoro-β-D-arabinofuranosyl)-5-iodouracil], and DNA from the clones analyzed by Southern blot. Targeted ES cell clones were injected into blastocysts (Matzuk et al., 1992).

Two of these cell lines were used to produce chimeric male mice that were fertile and transmitted the Zar1 mutant allele (Zar1^{tm1Zuk}, herein called Zar1⁻) to F1 progeny. Intercrossing of the F1 heterozygotes (FIG. 23B) yielded 232 F2 progeny [52 wild-type (22.5%), 119 heterozygous (Zar1^{+/-}) (51.5%), and 60 homozygous mutant (Zar1^{-/-}) mice (26.0%] from 32 litters analyzed. Thus, the mutated allele was transmitted with the expected Mendelian frequency of 1:2:1. The male (117):female (114) ratio was approximately 1:1.

Northern blot analysis with the full length Zar1 cDNA showed a significant reduction of the Zar1 mRNA in Zar1^{+/-} ovaries and failed to detect the 1.4 kb Zar1 mRNA in the ovaries of Zar^{-/-} mice (FIG. 23C), confirming that the Zar1^{tm1Zuk} allele was a null allele.

### Example 29

### Fertility of Zar1^{+/-} and Zar1^{-/-} Mice

Zar1^{+/-} and Zar1^{-/-} male and female mice showed no gross or histological abnormalities from birth through adult stages. The fertility of Zar1^{+/-} and Zar1^{-/-} mice was tested by mating over a 6 month period. Zar1^{-/-} males showed normal fertility (7.4±0.4 pups/litter). Since Zar1 was also expressed in the testis, this indicated that Zar1 was not essential for male fertility at a gross level.

Mating of 14 female Zar1^{+/-} mice with male Zar1^{+/-} mice resulted in 80 litters (0.95 litters/month/mouse) with an average litter size of 7.9±0.3 pups, which did not differ significantly from previous litter sizes of wild-type mice (8.4±0.2 pups/litter)(Kumar et al., 2000). Hence, Zar1^{+/-} females displayed normal fertility. In contrast, breeding of 20 Zar1^{-/-} female mice with control males failed to yield any offspring over 6 months. Thus, ZAR1 plays an essential role in female fertility.

### Example 30

### Subcellular Localization of ZAR1

To further define the function of ZAR1, immunostaining and indirect immunofluorescent labeling with ZAR1 antisera were used to evaluate protein expression and subcellular localization in oocytes and zygotes.

Briefly, immunohistochemistry was performed using ZAR1 antibodies. To prepare the ZAR1 antibodies, a partial mouse Zar1 cDNA [nucleotides 151-1056] was subcloned into pET23b vector, and fused recombinant ZAR1 protein (T7-tag at N-terminal and His-tag at C-terminal) was injected into goats to produce polyclonal antibodies (CoCalico Biologicals, Inc., Reamstown, Pa.). Next, immunostaining was performed using the primary antibody (diluted at 1:1,000) (Yan et al., 2002). Incubation with secondary antibody and visualization of positive cells were performed using the New Fuchsin kit (BioGenex, San Ramon, Calif.). Preimmune serum was used in control sections.

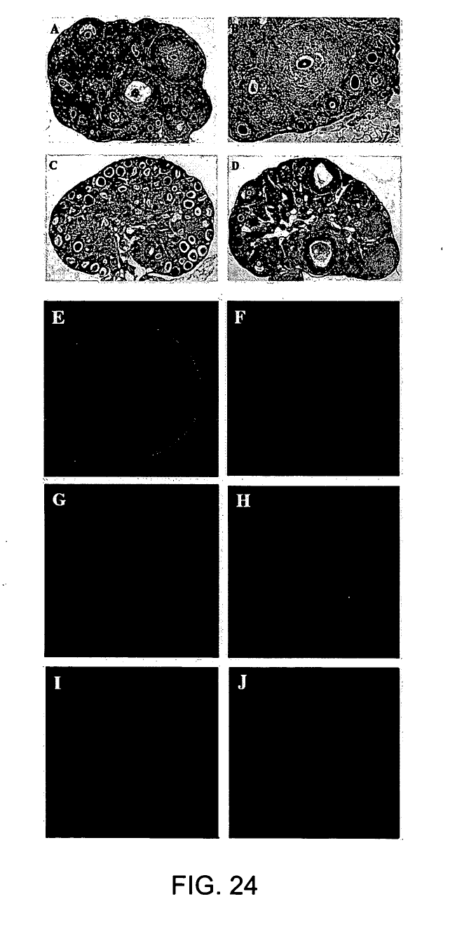

The ZAR1 protein localized predominantly to the cytoplasm of oocytes in both wild-type (FIGS. 24A-24B) and Gdf9^{-/-} ovaries (FIG. 17C). Consistent with the in situ hybridization analysis, ZAR1 protein was present from the primary through antral follicle stages (FIG. 24B).

Immunofluorescence analysis of oocytes and embryos was performed (Yan et al., 2002) to evaluate protein expression. Reaction with the ZAR1 goat antisera (diluted 1/1000 in block solution) was carried out for 1 h, followed by exposure to 3 μg/ml of FITC-conjugated anti-rabbit IgG (Jackson Immuno Research Laboratories, West Grove Pa.) for 45 min. DNA was labeled with propidium iodide (10 μg/ml, for 10 min). Negative control samples were evaluated with pre-immune serum.

Moreover, ZAR1 protein was distributed diffusely throughout the cytoplasm of fully-grown oocytes isolated from Zar1^{+/-} mice (FIG. 24E), and consistent with the above Northern blot, ovaries (FIG. 24D) and oocytes (FIG. 24F) from Zar1^{-/-} females exhibited no protein. ZAR1 was also detected, after the resumption of meiosis and progression to metaphase-I (FIG. 24G) and metaphase-II (FIG. 24H).

Next, fertilized zygotes, which failed to undergo the first mitotic division by 24 h post-fertilization, were evaluated to determine chromatin and microtubule configurations. The zygotes were fixed, permeabilized, and blocked as indicated. All subsequent steps, including rinses, were carried out at 37° C. in block solution. Microtubules were labeled with anti-β-tubulin (3.8 μg/ml, for 1 h) and a FITC-conjugated anti-mouse secondary antibody (1.3 μg/ml, for 45 min), while DNA was labeled with propidium iodide (10 μg/ml, for 10 min). Fluorescence was detected using a TCS-NT laser scanning confocal microscope equipped with an air-cooled argon ion laser system (Leica Microsystems).

ZAR1 persisted in the cytoplasm of early 1-cell zygotes post-fertilization (FIG. 19I) but was dramatically reduced in 2-cell embryos (FIG. 24J). Thus, ZAR1 functions at any stage of oogenesis from the primary follicle stage through the formation of 2-cell embryos. The rapid disappearance of ZAR1 at the 2-cell stage, however, suggested a critical role in the oocyte-to-embryo transition.

### Example 31

### In Vitro Oocyte Maturation and Fertilization

Sexually mature, heterozygous control and Zar1^{-/-} female mice were injected with 5 IU of PMSG to stimulate preovulatory follicle development. Cumulus-enclosed oocyte complexes were isolated 48 h later and cultured for 17 h in Minimal Essential Medium with 5% serum. The surrounding somatic cells were subsequently removed, and the oocytes were examined to determine the progression of meiosis. Mature MII-stage eggs were fertilized in vitro with sperm from wild type (C57BL/6J×SJL/J) F1 mice 19. Development of zygotes and 2-cell stage embryos was assessed at 6 and 24 h post-fertilization, respectively. Blastocyst formation was evaluated on day 5.

### Example 32

### Zar1 Embryonic Development

To confirm that ovulation was occurring and to further study the cause of the infertility and subfertility of the Zar1^{-/-} and Zar1^{+/-} mice, pharmacological superovulation of Zar1^{-/-}, and Zar1^{+/-} mice was performed and the eggs were collected from the oviducts and cultured in vitro.

Briefly, Twenty-five-day-old Zar1^{+/-} and Zar1^{-/-} female mice were injected with PMSG (i.p., 5.0 IU/mouse), and given hCG (i.p., 5 IU/mouse) 48 h later. Mice were then caged overnight with (C57BL/6J×129S6/SvEv)F1 stud males. The following morning, eggs and/or embryos were recovered in M2 medium, counted, and cultured in vitro up to 4 days in M16 medium (Sigma, St. Louis, Mo.). Alternatively, adult mutant females were mated to stud males, uteri and oviducts flushed on day 3.5, and embryos collected and cultured in M16 medium.

Superovulation with exogenous gonadotropins demonstrated similar numbers of oocytes from Zar1^{-/-} (34.31±4.12; n=14) and Zar1^{+/-} (31.63±4.78; n=8) females. Yet further, the majority of Zar1^{+/-} and Zar1^{-/-} oocytes resumed meiosis and progressed to metaphase-II during a 17-h culture.

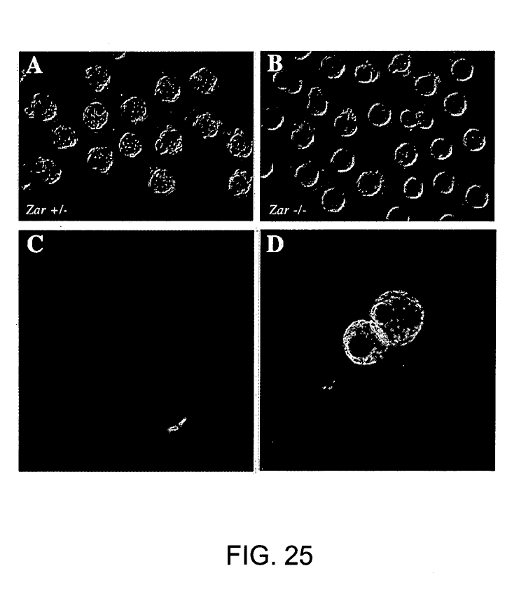

Next, metaphase-II oocytes were fertilized in vitro or after mating with stud males, embryos were recovered from the reproductive tract and cultured for up to 4 days or from adult females on day 3.5 (Table 7). Most oocytes from Zar1^{-/-} females formed two distinct pronuclei within 8 h post-insemination similar to the controls. However, while the first cleavage occurs in 89.3% of in vivo fertilized embryos from Zar1^{+/-} females and 86.5% of in vitro fertilized embryos from Zar1^{+/-} females, “apparent” 2-cell embryos (some of which appeared fragmented) were observed in 20.8% of in vivo fertilized embryos and 19.1% of in vitro fertilized embryos from Zar1^{-/-} females. Most 2-cell embryos from Zar1^{+/-} mice progressed to the morula and blastocyst stages by the fourth day of culture (Table 7); however, embryos from Zar1^{-/-} mice either remained at the one- or 2-cell stage or degenerated. Whereas 100% of the embryos isolated from the uteri of adult Zar1^{+/-} females developed to blastocysts by day 3.5, only fragmented, 1-cell, and a single 2-cell embryo could be observed in the Zar1^{-/-} females (FIGS. 25A-25B and Table 6). Therefore, an arrest of early embryo cleavage at the zygote stage accounts for the infertility of Zar1^{-/-} females.

TABLE 6
Evaluation of in vitro and in vivo oocyte maturation and embryo development.

| | | | Total Oocytes/ | | | | |
|---|---|---|---|---|---|---|---|
| Age | Genotype | N | embryos | % MII-stage | % 2 pronuclei | % 2-Cell | % Blastocyst |
| 3-4 wk | +/− | 6 | 156 | 74.4 ± 5.5 | 92.9 ± 2.1 | 86.5 ± 1.4 | 82.1 ± 1.5 |
| 3-4 wk | −/− | 5 | 137 | 62.9 ± 4.3 | 82.4 ± 7.5 | 19.1 ± 9.1 | 0.0 |
| Adult | +/− | 6 | 41 | — | — | 0.0 | 100 |
| Adult | −/− | 9 | 19 | — | — | 5.3 | 0.0 |

### Example 33

### DNA Synthesis

In further determining the cause of the infertility of the Zar1^{-/-} mice, DNA synthesis was performed. Briefly, fully-grown oocytes from Zar1^{+/-} and Zar1^{-/-} mice were in vitro matured and fertilized, as previously described. Approximately 8 h post-fertilization, the zygotes that had formed a male and female pronucleus (PN) were transferred to media supplemented with 50 μM bromo-deoxyuridine (BrdU) for overnight culture. At 24 h post-fertilization, embryos were fixed and processed to assess BrdU incorporation (Ferreira et al., 1997). Fluorescence was detected using a confocal microscope.

Analysis of the timing of the embryonic block showed that the arrested zygotes from Zar1^{-/-} females progressed through G1 and successfully entered S-phase. The chromatin of both maternal and paternal pronuclei was completely decondensed (FIG. 25C). BrdU was readily incorporated into both pronuclei of embryos from Zar1^{-/-} females (FIG. 25D), indicating active DNA synthesis in S-phase. The microtubule network showed an interphase configuration with no assembled spindle apparatus. In vitro fertilized oocytes were treated with colcemid, a reagent that depolymerized microtubules to arrest cells at M-phase. As expected, all of the zygotes derived from Zar1^{+/-} females arrested at M-phase after colcemid treatment. Only a few zygotes from Zar1^{-/-} females were similarly arrested; this corresponded to the number of 2-cell embryos normally observed in this group. Hence, the small percent of fertilized oocytes from Zar1^{-/-} females that progressed to the 2-cell stage entered M-phase, yet the majority arrested earlier, presumably at the S/G2 transition or the G2 stage of the first meiotic division. Thus, the maternal and paternal genomes remain separated in discrete pronuclei, and the two haploid genomes failed to unite, indicating that the completion of fertilization has not occurred.

### Example 34

### Yeast Two-Hybrid Screening

Yeast Two hybrid screen was used to elucidate or characterize the function of a protein by identifying other proteins with which it interacts.

The full-length open reading frame of mouse Zar1 was subcloned into the pGBKT7 vector for expression as a GAL4 DNA binding fusion protein. Ovarian and oocyte cDNA libraries were subcloned into the pGADT7 vector to be expressed as transactivation domain fusion proteins. In this yeast two-hybrid system, interactions between ZAR1 and proteins encoded by library cDNAs are expected to reconstitute transactivating complexes, which bind to GAL4 DNA and promote transcription of selectable markers. To identify ZAR1-interacting proteins, ovary cDNA transformants were screened by mating. Colonies grew on Leu-/Trp-/Ade-/His-/X-alpha-Gal selection plates and certain isolated plasmids with inserts were sequenced. Four of these sequences corresponded to Polr2c (DNA directed RNA polymerase II polypeptide C), Gnb2 (Guanine nucleotide binding protein, beta 2), Polr2g (DNA directed RNA polymerase II polypeptide G), and Lmo1 (LIM only 1).

### Example 35

### Cell-Free Transcription/Translation of Zar1

Cell-free in vitro transcription/translation of Zar1 was performed to confirm in vitro protein interaction. Briefly, the pGBKT7 (MYC-Tagged) and pGADT7 (HA-Tagged) vectors were used as templates for in vitro transcription/translation using [³⁵S] Met and the TNT T7 Coupled Reticulocyte Lysate System (Promega, Madison, Wis.). In vitro translated proteins were combined at room temperature for 1 h, and reciprocal co-immunoprecipitation experiments were performed using mouse anti-MYC monoclonal or rabbit anti-HA polyclonal antibodies (Clontech).

Cell-free in vitro transcription/translation of Zar1, Polr2c (DNA directed RNA polymerase II polypeptide C), Gnb2 (Guanine nucleotide binding protein, beta 2), Polr2g (DNA directed RNA polymerase II polypeptide G), and Lmo1 (LIM only 1) cDNAs was performed. The cDNAs for Polr2c, Gnb2 and Lmo11 were inserted into the pGADT7 (HA-Tagged) vector and Zar1 cDNA was inserted into the pGBKT7 (MYC-Tagged) vector. The in vitro translated proteins were then co-immunoprecipitated and analyzed on a SDS-PAGE.

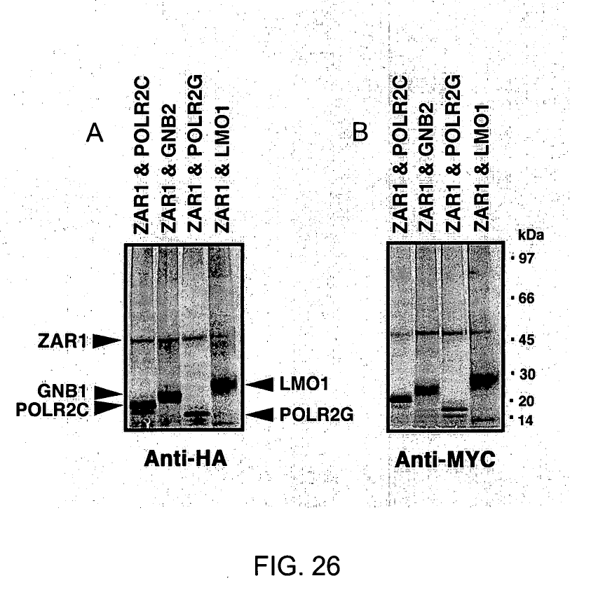

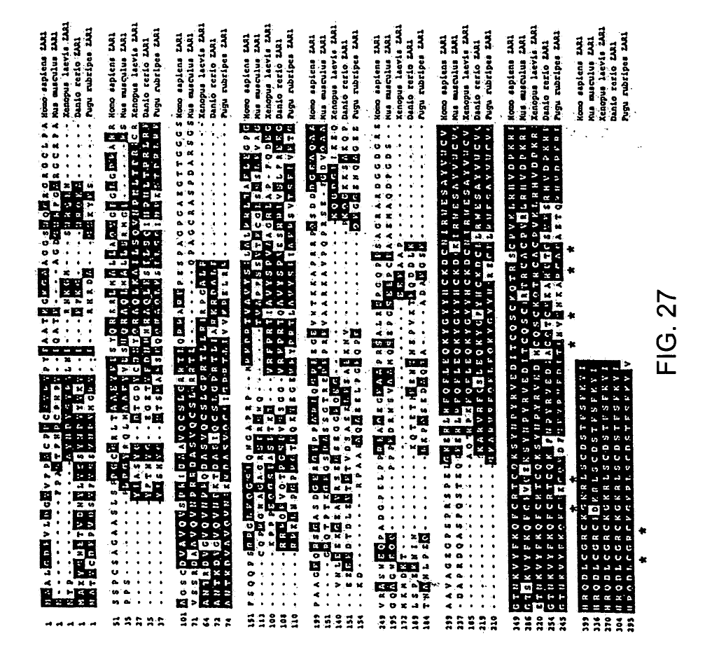

FIGS. 26A and 27B demonstrates that POLR2C, GNB2, POLR2G, and LMO1 bind to the ZAR1.

### Example 36

### ZAR1 Interactions in CHO Cells

To confirm that ZAR1 binds to POLR2C, GNB2, POLR2G, and LMO1, co-immunoprecipitation studies are performed using extracts of transiently transfected Chinese hamster ovary (CHO) cells.

Briefly, CHO-K1 cells (American Type Culture Collection, Manassas, Va.) are cultured in Dulbecco's modified Eagle's medium/Ham's F-12 (DMEM/F-12) containing 10% fetal bovine serum (FBS) and are grown to 90-95% confluence in 6 cm dishes. To express tagged proteins, mouse cDNAs encoding the open reading frames of ZAR1, POLR2C, GNB2, POLR2G, and LMO1 are inserted into pCMV-Tag4A/FLAG-C and pCMV-Tag5A/MYC-C vectors (Stratagene, La Jolla, Calif.) and are transiently transfected using LipofectAMINE 2000 (Invitrogen Life Technologies). Twenty-four hours after transfection, cells are harvested, lysed in lysis buffer [50 mM Tris HCl, pH 7.4, 150 mM NaCl, 1 mM EDTA, 1% Triton X-100 and protease inhibitor cocktail (Sigma, Saint Louis, Mo.)] and are analyzed by immunoprecipitation and SDS-PAGE.

The MYC-tagged constructs are detected with anti-MYC antibodies and FLAG-tagged constructs are detected with anti-FLAG antibodies.

### Example 37

### Conformation of Binding of the Binding Partners

To confirm that ZAR1 binds to POLR2C, GNB2, POLR2G, and LMO1, ovarian protein is isolated from mice. Next, immunoprecipitation experiments are performed using ZAR1 antibodies. Western blot analysis is performed using antibodies to POLR2C, GNB2, POLR2G, and LMO1.

### Example 38

### Generation of Knockout Mice Lacking Novel Ovary-Expressed Genes

Using the gene sequence obtained above, the inventors generate a targeting vector to mutate the O1-184 gene in embryonic stem (ES) cells. This targeting vector is electroporated into the hprt-negative AB2.1 ES cell line and selected in HAT and FIAU. Clones are processed for Southern blot analysis and screened using 5′ and 3′ external probes. ES cells with the correct mutation are injected into blastocysts to generate chimeras and eventually heterozygotes and homozygotes for the mutant O1-184 gene.

Since expression of O1-184 was limited to the ovary, the inventors anticipate that these O1-184-knockout mice are viable, but that females lacking this gene product can have fertility alterations (i.e., be infertile, subfertile, or superfertile). Mutant mice are analyzed for morphological, histological and biochemical information relating to intraovarian proteins required for folliculogenesis, oogenesis, or fertilization using techniques well within the ability of the person of ordinary skill in the art. It is envisioned that the absence of this protein can result in female mice having increased or decreased fertility. These studies will lead a search for human reproductive conditions with similar idiopathic phenotypes.

### Example 39

### Generation of O1-184 Transgenic Animals

The O1-184 gene is flanked by genomic sequences and is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. These animals are generated to overexpress O1-184 or express a mutant form of the polypeptide.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### REFERENCES

The following references, to the extent that they provide exemplary procedural or other details supplementary to those set forth herein, are specifically incorporated herein by reference:
Albrecht, U., et al., (1997). In Molecular and Cellular Methods in Developmental Toxicology, G. P. Daston, ed. (Boca Raton, Fla., CRC Press), pp. 23-48.
Bouniol-Baly et al., (1999) Biol Reprod 60, 580-7.
Burglin et al., (1987) Genes Dev 1, 97-107.
Capecchi, (1994) Scientific American 270, 52-59.
Carabatsos M., et al., (1998). Dev. Biol. 203, 373-384.
Chan et al., (1989) Biochemistry 28, 1033-9.
Chan, W. Y., et al., (1989). Biochemistry 28, 1033-9.
Channing, C. P., (1970). Recent Prog. Horm. Res. 26, 589-622.
Christians et al., (2000) Nature 407, 693-4.
Cotten, M., et al., (1986). Biochemistry 25, 5063-5069.
Crevel, G., et al., (1997). J Struct Biol 118, 9-22.
Dilworth et al., (1987) Cell 51, 1009-1018.
Dimitrov, S., and Wolffe, A. (1996). EMBO Journal 15, 5897-5906.
Dingwall et al., (1987) EMBO J. 6, 69-74.
Dong, J., et al., (1996). Nature 383, 531-535.
Dube, J. L., et al., (1998). Molecular Endocrinology 12, 1809-1817.
Earnshaw, W., et al., (1980). Cell 21, 373-383.
El-Fouly, M. A., et al., 1970. Endocrinology 87, 288-293.
Elvin, J. A., and Matzuk, M. M. (1998). Reviews of Reproduction 3, 183-195.
Elvin, J. A., et al., (1999). Mol Endocrinol 13, 1018-34.
Elvin, J. A., et al., (2000). Mol Cell Endocrinol 159, 1-5.
Elvin, J. A., et al., 1999B. Mol. Endocrinol. 13, 1035-1048.
Elvin, J. A., et al., 2000. Proc. Natl. Acad. Sci. USA., 97: 10288-10293.
Gillespie et al., (2000) Nucleic Acids Res 28, 472-80.
Gurtu et al., (2002) Genetics 160, 271-7.
Hogan, B., et al., (1994). Manipulating the mouse embryo—a laboratory manual (Plainview, Cold Spring Harbor Laboratory Press).
Howell et al., (2001) Cell 104, 829-38.
Hunt et al., (2002) Science 296, 2181-3.
Ito, T., Tyler, et al., (1996). J Biol Chem 271, 25041-8.
Iwata, K., et al., (1999). Int J Biol Macromol 26, 95-101.
Krohne, G., and Franke, W. (1980a). Proc Natl Acad Sci 77, 1034-1038.
Kumar, T. (1994) Human Rep 9, 578-585.
Kumar, T. R., et al., (1997). Nature Genetics 15, 201-204.
Laskey, R., et al., (1993). Philos Trans R Soc Lond B Biol Sci 339, 263-269.
Latham et al., (1992) Dev Biol 149, 457-62.
Leno, G., et al., (1996). J Biol Chem 271, 7253-7256.
Ma et al., (2001) Biol Reprod 64, 1713-21.
MacArthur et al., (1997) Genomics 42, 137-140 (1997).
Maeda et al., (1998). Zygote 6, 39-45.
Mahmoudi, M., and Lin, V. K. (1989). Biotechniques 7, 331-332.
Mattson et al., (1990) Mol. Reprod. Dev. 25, 374-383.
Matzuk et al., (2002) Science 296, 2178-2180 (2002).
Matzuk, et al., (1992). Nature 360, 313-319.
Matzuk, M. M., et al., (1995). Nature 374, 356-360.
Matzuk, M. M., et al., (1996). Recent Prog Horm Res 51, 123-54.
McGrath, S. A., et al., (1995). Molecular Endocrinology 9, 131-136.
McLay, D., and Clarke, H. (1997). Dev Biol 186, 73-84.
Meric et al., (1997) J Biol Chem 272, 12840-12846.
Mills, A., et al., (1980). J Mol Biol 139, 561-568.
Nishimori, K., and Matzuk, M. M. (1996). Reviews of Reproduction 1, 203-212.
Ohsumi et al., (1991) Dev Biol 148, 295-305.
Pedersen, T., and Peters, H. (1968). Journal of Reproduction and Fertility 17, 555-557.
Perreault, (1992) Mutat Res 296, 43-55.
Philpott et al., (1991) Cell 65, 569-578
Philpott, A., and Leno, G. (1992). Cell 69, 759-767.
Robbins et al., (1991) Cell 64, 615-623.
Schmidt-Zachmann et al., (1988) Chromosoma 96, 417-26.
Sealy, L., et al., Biochemistry 25, 3064-3072.
Senaphthy, P., et al., Methods Enzymol 183, 252-278.
Service, R. (1996). Science 272, 1258.
Tong et al., (2000) Nat Genet 26, 267-8.
Usui, (1976) Ultrastruct Res 57, 276-88.
Vancurova et al., (1995). J Cell Sci 108, 779-787.
Vanderhyden et al., (1993) Endocrinology 133, 423-426.
Wickramasinghe et al., (1991) Dev Biol 143, 162-72.
Wu et al., (2003) Nature Genetics 33, 187-191.
Zuccotti et al., (1998) Biol Reprod 58, 700-4.
Zuccotti et al., J Endocrinol Invest 23:623-9.

What is claimed is:

1. An isolated polynucleotide sequence comprising a nucleic acid sequence selected from the group consisting of SEQ.ID.NO.11, SEQ.ID.NO.13, SEQ.ID.NO.12, SEQ.ID.NO.28, SEQ.ID.NO.30, SEQ.ID.NO.31, SEQ.ID.NO.33, SEQ.ID.NO.35, SEQ.ID.NO.37, SEQ.ID.NO.38, SEQ.ID.NO.40, SEQ.ID.NO.41, and SEQ.ID.NO.43.

2. An isolated polynucleotide sequence encoding a protein, wherein said protein is selected from the group consisting of:
(a) a polynucleotide sequence encoding SEQ.ID.NO.16, SEQ.ID.NO.29, SEQ.ID.NO.32, SEQ.ID.NO.34, SEQ.ID.NO.36, SEQ.ID.NO.39, or SEQ.ID.NO.42;
(b) a polynucleotide sequence encoding an amino acid sequence having at least 40% identity with SEQ.ID.NO.16, SEQ.ID.NO.29, SEQ.ID.NO.32, SEQ.ID.NO.34, SEQ.ID.NO.36, SEQ.ID.NO.39, or SEQ.ID.NO.42,
(c) an isolated nucleic acid molecule that hybridizes with the polynucleotide sequence of (a) under hybridization conditions of 0.02 M to about 0.15 M NaCl at temperatures of about 50° C. to about 70° C.; and
(d) an isolated polynucleotide sequence that is complementary to (a), (b) or (c).

3. An expression cassette comprising the polynucleotide sequence of claim 1 or 2 operatively linked to a promoter sequence.

4. A vector comprising the expression cassette of claim 3.

5. An isolated polypeptide sequence comprising an amino acid sequence of SEQ.ID.NO.16, SEQ.ID.NO.29, SEQ.ID.NO.32, SEQ.ID.NO.34, SEQ.ID.NO.36, SEQ.ID.NO.39 or SEQ.ID.NO.42.

6. An isolated polypeptide encoded by the polynucleotide sequence of claim 1 or 2.

7. A monoclonal antibody that specifically binds immunologically the polypeptide of claim 5.

8. A monoclonal antibody that specifically binds immunologically the polypeptide of claim 6.

9. A polyclonal antiserum, antibodies which binds immunologically to the polypeptide of claim 5.

10. A polyclonal antiserum, antibodies which binds immunologically to the polypeptide of claim 6.

11. A hybridoma cell that produces a monoclonal antibody that binds immunologically to the polypeptide of claim 5.

12. A hybridoma cell that produces a monoclonal antibody that binds immunologically to the polypeptide of claim 6.

13. A composition comprising the antibody of claim 7, 8, 9 or 10.

14. A host cell comprising the expression cassette of claim 3.

15. The host cell of claim 14, wherein the cell is a eukaryotic cell or a prokaryotic cell.

16. A transgenic animal comprising the polynucleotide sequence of claim 1 or 2.

17. The transgenic animal, wherein the animal is a rodent, a mouse or a rat.

18. A transgenic animal comprising a polynucleotide sequence selected from the group consisting of SEQ.ID.NO.11, SEQ.ID.NO.13, SEQ.ID.NO.12, SEQ.ID.NO.28, SEQ.ID.NO.30, SEQ.ID.NO.31, SEQ.ID.NO.33, SEQ.ID.NO.35, SEQ.ID.NO.37, SEQ.ID.NO.38, SEQ.ID.NO.40, SEQ.ID.NO.41, and SEQ.ID.NO43.

19. A pharmaceutical composition comprising a modulator of O1-180 expression dispersed in a pharmaceutically acceptable carrier.

20. The composition of claim 19, wherein the modulator suppresses transcription of an O1-180 gene.

21. The composition of claim 19, wherein the modulator enhances transcription of an O1-180 gene.

22. The composition of claim 19, wherein the modulator is a polypeptide.

23. The composition of claim 19, wherein the modulator is a small molecule.

24. The composition of claim 19, wherein the modulator is a polynucleotide sequence.

25. The composition of claim 24, wherein the polynucleotide sequence is DNA or RNA.

26. The composition of claim 24 further comprising an expression vector, wherein the expression vector comprises a promoter and the polynucleotide sequence, operatively linked.

27. A pharmaceutical composition comprising a modulator of O1-180 activity dispersed in a pharmaceutically acceptable carrier.

28. The composition of claim 27, wherein the composition inhibits O1-180 activity.

29. The composition of claim 27, wherein the composition stimulates O1-180 activity.

30. A method of modulating contraception comprising administering to an animal an effective amount of a modulator of O1-180 activity or O1-180 expression dispersed in a pharmacologically acceptable carrier, wherein said amount is capable of decreasing conception.

31. A method of enhancing fertility comprising administering to an animal an effective amount of a modulator of O1-180 activity or O1-180 expression dispersed in a pharmacologically acceptable carrier, wherein said amount is capable of increasing conception.

32. The method of claim 30 or 31, wherein the animal is female.

33. The method of claim 30 or 31, wherein the animal is male.

34. A method of screening for a modulator of O1-180 expression comprising the steps of:
providing a cell expressing a O1-180 polypeptide;
contacting said cell with a candidate modulator;
measuring O1-180 expression; and
comparing said O1-180 expression in the presence of said candidate modulator with the expression of O1-180 in the absence of said candidate modulator; wherein a difference in the expression of O1-180 in the presence of said candidate modulator, as compared with the expression of O1-180 in the absence of said candidate modulator, identifies said candidate modulator as a modulator of O1-180 expression.

35. A method of identifying compounds that modulate the activity of O1-180 comprising the steps of:
obtaining an isolated O1-180 polypeptide or functional equivalent thereof;
admixing the O1-180 polypeptide or functional equivalent thereof with a candidate compound; and
measuring an effect of said candidate compound on the activity of O1-180.

36. A method of screening for a compound which binds with O1-180 comprising:
exposing a O1-180 protein, or a fragment thereof to a candidate compound; and
determining whether said compound binds to the O1-180 protein or fragment therof.

37. A method of identifying a compound that modulates O1-180 activity comprising
(a) providing a transgenic animal having (1) one or more regulatable O1-180 genes, (2) a knock-out of one or more O1-180 genes, or (3) a knock-in of one or more O1-180 genes;
(b) providing a control animal respectively for transgenic animal in step (a);
(c) exposing the transgenic animal and control animal to a potential O1-180-modulating compound and
(d) comparing the transgenic animal and the control animal group and determining the effect of the O1-180-modulating compound on the infertility or fertility in the transgenic animal as compared to the control animal.

38. A method for detecting the binding interaction of a first peptide and a second peptide of a peptide binding pair, comprising:
(i) culturing at least one eukaryotic cell under conditions to detect a selected phenotype or the absence of such phenotype, wherein the cell comprises;
a) a nucleotide sequence encoding a first heterologous fusion protein comprising the first peptide or a segment thereof joined to a DNA binding domain of a transcriptional activation protein;
b) a nucleotide sequence encoding a second heterologous fusion protein comprising the second peptide or a segment thereof joined to a transcriptional activation domain of a transcriptional activation protein; wherein binding of the first peptide or segment thereof and the second peptide or segment thereof reconstitutes a transcriptional activation protein; and
c) a reporter element activated under positive transcriptional control of the reconstituted transcriptional activation protein, wherein expression of the reporter element prevents exhibition of a selected phenotype;
(ii) detecting the ability of the test peptide to interact with O1-180 by determining whether the test peptide affects the expression of the reporter element which prevents exhibition of the selected phenotype, wherein said first or second peptide is an O1-180 peptide and the other peptide is a test peptide.

39. A method of identifying binding partners for O1-180 comprising the steps of:
exposing the protein to a potential binding partner; and
determining if the potential binding partner binds to O1-180.

40. A pharmaceutical composition comprising a modulator of O1-236 expression dispersed in a pharmaceutically acceptable carrier.

41. The composition of claim 40, wherein the modulator suppresses transcription of an O1-236 gene.

42. The composition of claim 40, wherein the modulator enhances transcription of an O1-236 gene.

43. The composition of claim 40, wherein the modulator is a polypeptide.

44. The composition of claim 40, wherein the modulator is a small molecule.

45. The composition of claim 40, wherein the modulator is a polynucleotide sequence.

46. The composition of claim 45, wherein the polynucleotide sequence is DNA or RNA.

47. The composition of claim 45 further comprising an expression vector, wherein the expression vector comprises a promoter and the polynucleotide sequence, operatively linked.

48. A pharmaceutical composition comprising a modulator of O1-236 activity dispersed in a pharmaceutically acceptable carrier.

49. The composition of claim 48, wherein the composition inhibits O1-236 activity.

50. The composition of claim 48, wherein the composition stimulates O1-236 activity.

51. A method of modulating contraception comprising administering to an animal an effective amount of a modulator of O1-236 activity dispersed in a pharmacologically acceptable carrier, wherein said amount is capable of decreasing conception.

52. A method of enhancing fertility comprising administering to an animal an effective amount of a modulator of O1-236 activity dispersed in a pharmacologically acceptable carrier, wherein said amount is capable of increasing conception.

53. The method of claim 51 or 52, wherein the animal is female.

54. The method of claim 51 or 52, wherein the animal is male.

55. A method of screening for a modulator of O1-236 expression comprising the steps of:
providing a cell expressing an O1-236 polypeptide contacting said cell with a candidate modulator;
measuring O1-236 expression; and
comparing said O1-236 expression in the presence of said candidate modulator with the expression of O1-236 in the absence of said candidate modulator; wherein a difference in the expression of O1-236 in the presence of said candidate modulator, as compared with the expression of O1-236 in the absence of said candidate modulator, identifies said candidate modulator as a modulator of O1-236 expression.

56. A method of identifying compounds that modulate the activity of O1-236 comprising the steps of:
obtaining an isolated O1-236 polypeptide or functional equivalent thereof;
admixing the O1-236 polypeptide or functional equivalent thereof with a candidate compound; and
measuring an effect of said candidate compound on the activity of O1-236.

57. A method of identifying binding partners for O1-180 comprising the steps of:
exposing the protein to a potential binding partner; and
determining if the potential binding partner binds to O1-180.

58. A method of identifying a compound that modulating O1-236 activity comprising
(a) providing a transgenic animal having (1) one or more regulatable O1-236 genes, (2) a knock-out of one or more O1-236 genes, or (3) a knock-in of one or more O1-236 genes;
(b) providing a control animal respectively for transgenic animal in step (a); and
(c) exposing the transgenic animal group and control animal group to a potential O1-236-modulating compounds; and
(d) comparing the transgenic animal and the control animal and determining the effect of the compound on infertility or fertility in the transgenic animal as compared to the control animal.

59. A method of detecting a binding interaction of a first peptide and a second peptide of a peptide binding pair, comprising:
(i) culturing at least one eukaryotic cell under conditions suitable to detect the selected phenotype; wherein the cell comprises;
a) a nucleotide sequence encoding a first heterologous fusion protein comprising the first peptide or a segment thereof joined transcriptional activation domain of a transcriptional activation protein;
b) a nucleotide sequence encoding a second heterologous fusion protein comprising the second peptide or a segment thereof joined to a transcriptional activation protein transcriptional activation domain; wherein binding of the first peptide or segment thereof and the second peptide or segment thereof reconstitutes a transcriptional activation protein; and
c) a reporter element activated under positive transcriptional control of the reconstituted transcriptional activation protein, wherein expression of the reporter element produces a selected phenotype;
(ii) detecting the binding interaction of the peptide binding pair by determining the level of the expression of the reporter element which produces the selected phenotype;
wherein said first or second peptide is an O1-236 peptide and the other peptide is a test peptide.

60. A detecting the binding interaction of a first peptide and a second peptide of a peptide binding pair, comprising:
(i) culturing at least one yeast cell under conditions to detect a selected phenotype or the absence of such phenotype, wherein the yeast cell comprises;
a) a nucleotide sequence encoding a first heterologous fusion protein comprising the first peptide or a segment thereof joined to a DNA binding domain of a transcriptional activation protein;
b) a nucleotide sequence encoding a second heterologous fusion protein comprising the second peptide or a segment thereof joined to a transcriptional activation domain of a transcriptional activation protein; wherein binding of the first peptide or segment thereof and the second peptide or segment thereof reconstitutes a transcriptional activation protein; and
c) a reporter element activated under positive transcriptional control of the reconstituted transcriptional activation protein, wherein expression of the reporter element prevents exhibition of a selected phenotype;
(ii) detecting the ability of the test peptide to interact with O1-236 by determining whether the test peptide affects the expression of the reporter element which prevents exhibition of the selected phenotype, wherein said first or second peptide is an O1-236 peptide and the other peptide is a test peptide.

61. A method of identifying binding partners for O1-236 comprising the steps of:
exposing the protein to a potential binding partner; and
determining if the potential binding partner binds to O1-236.


SEQUENCE LISTING (43 sequences)

SEQ ID NO: 1 - DNA, 1258 bp, Mus musculus
ggcgggcgacgcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgaccgccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccgatcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgcgaggaaagcggtcccccagccgcgaagcgaggagggcgatgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctcgagaccaggcctccccgcaaagcacggagcaggacaaggagcgcctgcgtttccagttcttagagcagaagtacggctactatcactgcaaggactgcaaaatccggtgggagagcgcctatgtgtggtgtgtgcagggcaccagtaaggtgtacttcaaacagttctgccgagtgtgtgagaaatcctacaacccttacagagtggaggacatcacctgtcaaagttgtaaaagaactagatgtgcctgcccagtcagacttcgccacgtggaccctaaacgcccccatcggcaagacttgtgtgggagatgcaaggacaaacgcctgtcctgcgacagcaccttcagcttcaaatacatcatttagtgagagtcgaaaacgtttctgctagatggggctaatggaatggacaagtgacgtttctcccctcttcacctcttccctttccaaattcttcatgacagacagtattacttgagtataaagcctgtgaataaaaggtattgcaaacaa

SEQ ID NO: 2 - protein, 361 aa, Mus musculus
MFPASTFHPCPHPYPQATKAGDGWRFGARGCRPAPPSFLPGYRQLMAAEYVDSHQRAQLMALLSRMGPRSVSSRDAAVQVNPRRDASVQCSLGRRTLQPAGCRASPDARSGSCQPRGHAGAGRSPRSWQTVAPFSSVTFCGLSSSLEVAGGRQTPTKGEGSPASSGTREPEPREVAARKAVPQPRSEEGDVQAAGQAGWEQQPPPEDRNSVAAMQSEPGSEEPCPAAEMAQDPGDSDAPRDQASPQSTEQDKERLRFQFLEQKYGYYHCKDCKIRWESAYVWCVQGTSKVYFKQFCRVCEKSYNPYRVEDITCQSCKRTRCACPVRFRHVDPKRPHRQDLCGRCKDKRLSCDSTFSFKYII

SEQ ID NO: 3 - DNA, 1817 bp, Mus musculus
gtcacagctttcccctgcccgaatatggtgatctgtctccattgtccagatcaggatgattctttagaagaagtcacagaggaatgctattccccacccaccctccagaacctggcaattcagagtctactgagggatgaggccttggccatttctgctctcacggacctgccccagagtctgttcccagtaatttttgaggaggccttcactgatggatatatagggatcttgaaggccatgatacctgtgtggcccttcccatacctttctttaggaaagcagataaataattgcaacctggagactttgaaggctatgcttgagggactagatatactgcttgcacaaaaggttcaaaccagtaggtgcaaactcagagtaattaattggagagaagatgacttgaagatatgggctggatcccatgaaggtgaaggcttaccagatttcaggacagagaagcagccaattgagaacagtgctggctgtgaggtgaagaaagaattgaaggtgacgactgaagtccttcgcatgaagggcagacttgatgaatctaccacatacttgttgcagtgggcccagcagagaaaagattctattcatctattctgtagaaagctactaattgaaggcttaaccaaagcctcagtgatagaaatcttcaaaactgtacacgcagactgtatacaggagcttatcctaagatgtatctgcatagaagagttggcttttcttaatccctacctgaaactgatgaaaagtcttttcacactcacactagatcacatcataggtaccttcagtttgggtgattctgaaaagcttgatgaggagacaatattcagcttgatttctcaacttcccacactccactgtctccagaaactctatgtaaatgatgtcccttttataaaaggcaacctgaaagaatacctcaggtgcctgaaaaagcccttggagacactttgcatcagtaactgtgacctctcacagtcagacttggattgcctgccctattgcctgaatatttgtgaactcaaacatctgcatattagtgatatatatttatgtgatttactccttgagcctcttggttttctccttgagagagttggagataccctgaaaaccctggaattggattcatgttgtatagtggactttcagttcagtgccttgctgcctgccctaagccaatgttctcacctcagagaggtcactttctatgataatgatgtttctctgcctttcttgaaaacaacttctacaccacacagccctgctgagtcagctgatctatgagtgttaccctgcccctctagagtgctatgatgacagtggtgtaatactaacacacagattagaaagtttttgtcctgagcttctggatatactgagagccaaaagacagctccatagtgtctcctttcaaacaaccaaatgctctaaatgtggtgggtgctacatttatgatcggcatacccaatgttgccgttttgtggaactactataagcttgattgtgaaactgagaaatagaaacttagtattggggactgatgaaatcctaagtgaatgtccactgctaaatggagcatgaaaatgtcaatcacctaaaagtctgagatacacaggaaagtcaataacttcctctgagctggtgaatggatgttgcatctgtagaaagtatcaagcacttgtagtttgaatgtgttacaatagaagcaccattttatgagactggcccaatctgttgactgcatacaataaatctgttgacttattaaatttttaaaaaaaaaaaaaaaaaaaaaa

SEQ ID NO: 4 - protein, 426 aa, Mus musculus
MVICLHCPDQDDSLEEVTEECYSPPTLQNLAIQSLLRDEALAISALTDLPQSLFPVIFEEAFTDGYIGILKAMIPVWPFPYLSLGKQINNCNLETLKAMLEGLDILLAQKVQTSRCKLRVINWREDDLKIWAGSHEGEGLPDFRTEKQPIENSAGCEVKKELKVTTEVLRMKGRLDESTTYLLQWAQQRKDSIHLFCRKLLIEGLTKASVIEIFKTVHADCIQELILRCICIEELAFLNPYLKLMKSLFTLTLDHIIGTFSLGDSEKLDEETIFSLISQLPTLHCLQKLYVNDVPFIKGNLKEYLRCLKKPLETLCISNCDLSQSDLDCLPYCLNICELKHLHISDIYLCDLLLEPLGFLLERVGDTLKTLELDSCCIVDFQFSALLPALSQCSHLREVTFYDNDVSLPFLKTTSTPHSPAESADL

SEQ ID NO: 5 - DNA, 1018 bp, Mus musculus
gccatattgaggacctgcagtagaggtggaacccatgactggcagcgcaaacacagtgataacagctgagctccaagcaaggacccaggaccttgcctcaccacagacataatctttccccacaacacctccaccaagccgccctgtaaatcgacatgagtcgccacagcaccagcagcgtgaccgaaaccacagcaaaaaacatgctctggggtagtgaactcaatcaggaaaagcagacttgcacctttagaggccaaggcgagaagaaggacagctgtaaactcttgctcagcacgatctgcctgggggagaaagccaaagaggaggtgaaccgtgtggaagtcctctcccaggaaggcagaaaaccaccaatcactattgctacgctgaaggcatcagtcctgcccatggtcactgtgtcaggtatagagctttctcctccagtaacttttcggctcaggactggctcaggacctgtgttcctcagtggcctggaatgttatgagacttcggacctgacctgggaagatgacgaggaagaggaggaagaggaggaggaagaggatgaagatgaggatgcagatatatcgctagaggagatacctgtcaaacaagtcaaaagggtggctccccagaagcagatgagcatagcaaagaaaaagaaggtggaaaaagaagaggatgaaacagtagtgaggcccagccctcaggacaagagtccctggaagaaggagaaatctacacccagagcaaagaagccagtgaccaagaaatgacctcatcttagcatcttctgcgtccaaggcaggatgtccagcagctgtgttttggtgcaggtgtccagccccaccaccctagtctgaatgtaataaggtggtgtggctgtaaccctgtaacccagccctccagtttccggaggtttttggtgaagagcccccagcaagttcgcctagggccacaataaaatttgcatgatcaggaaaaaaaaaaaaaaaaaaaaaaaaaaaaaaaaaaa

SEQ ID NO: 6 - protein, 207 aa, Mus musculus
MSRHSTSSVTETTAKNMLWGSELNQEKQTCTFRGQGEKKDSCKLLLSTICLGEKAKEEVNRVEVLSQEGRKPPITIATLKASVLPMVTVSGIELSPPVTFRLRTGSGPVFLSGLECYETSDLTWEDDEEEEEEEEEEDEDEDADISLEEIPVKQVKRVAPQKQMSIAKKKKVEKEEDETVVRPSPQDKSPWKKEKSTPRAKKPVTKK

SEQ ID NO: 7 - DNA, 6970 bp, Mus musculus
  misc_feature (1)..(6970): n equals unknown
acagcagaggtgatgctcagaaatcaagttttaacagagggccaggtgcttctagagtaggaggggattgcacacctccccaccccctcctctttcccaggcttcttaacagcctgctgtgggaagctgacccttagatggagccctgaagccatattgaggacctgcagtagaggtggaacccatgactggcagcgcagtaagcttgagcaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctttgcattactcagaacacagtgataacagctgagctccaagcaaggacccaggaccttgcctcaccacagacataatctttccccacaacacctccaccaagccgccctgtaaatcgacatgagtcgccacagcaccagcagcgtgaccgaaaccacagcaaaaaacatgctctggggtaagggctaaggctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtcttcgctgtgcaggtagtgaactcaatcaggaaaagcagacttgcacctttagaggccaatgcgagaagaaggacagctgtaaactcttgctcagcacggtgggtgtctcccaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatcacctttctcagatctgcctgggggagaaagccaaagaggaggtgaaccgtgtggaagtcctctcccaggaaggcagaaaaccaccaatcactattgctacgctgaaggcatcagtcctgcccatggtgagtcttctctccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaagggggacacaggtcactgtgtcaggtatagagctttctcctccagtaacttttcggctcaggactggctcaggacctgtgttcctcagtggcctggaatgttatggtaagttgtagcctannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggctacccattccagagacttcggacctgacctgggaagatgacgaggaagaggaggaagaggaggaggaagaggatgaagatgaggatgcagatatatcgctagaggagatacctgtcaaacaagtcaaaagggtggctccccagaagcagatgagcatagcaaaggtggggggaaaagaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggtttttgttccagaaaaagaaggtggaaaaagaagaggatgaaacagtagtgaggtaattcatgcagttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctattccctttccaggcccagccctcaggacaagagtccctggaagaaggtgagcaataagaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcttatctgcacaggagaaatctacacccagagcaaagaagccagtgaccaagaaatgacctcatcttagcatcttctgcgtccaaggcaggatgtccagcagctgtgttctggtgcaggtgtccagccccaccaccctagtctgaatgtaataaggtggtgtggctgtaaccctgtaacccagccctccagtttccggaggtttttggtgaagagcccccagcaagttcgcctagggccacaataaaatttgcatgatcaggacctccctctgcctccccctccctggatgggtctcctcgctgctgcgatagctcatgtgcccagcagagggcaaccacgagcaagaaaccagccccatgt

SEQ ID NO: 8 - DNA, 1207 bp, Human
agggggcgccaggaggcctcggcgggtccgcaattggccgggacagcttctcacgaaaggtcctgggccggcatcatcagcctcacctgggaactggttagaactacaaattccctcggccccacccagaccgacgccaagggcagctgtggagtggggcgcggcaatgcgccccttaacagccctccaggcttcttagcccgggcttggacagccgccttccggccagaggggatgaggttgcgctgcgctccgggagcgccgatggcgtgactggccccgcgcggagcagcgacactgcccggccagcccgcttctctgcccggagccatgaatctcagtagcgccagtagcacgaggaaaaggcagtgacgaccgtgctctggggctgcgagctcagtcaggagaggcggacttggaccttcagaccccagctggaggggaagcagagctgcaggctgttgcttcatacgatttgcttgggggagaaagccaaagaggagatgcatcgcgtggagatcctgcccccagcaaaccaggaggacaagaagatgcagccggtcaccattgcctcactccaggcctcagtcctccccatggtctccatggtaggagtgcagctttctcccccagttactttccagctccgggctggctcaggacccgtgttcctcagtggccaggaacgttatgaagcatcagacctaacctgggaggaggaggaggaagaagaaggggaggaggaggaagaggaagaggaagatgatgaggatgaggatgcagatatatctctggaggagcaaagccctgtcaaacaagtcaaaaggctggtgccccagaagcaggcgagcgtggctaagaaaaaaaagctggaaaaagaagaagaggaaataagagccagcgttagagacaagagccctgtgaaaaaggccaaagccacagccagagccaagaagccaggattcaagaaatgaggagccacgccttggggggcacggtgcaaagtgggccttccctgggctgtgctgcaggcacagggtgcccctgtccagcccctccacctgtgtctgaatgcaacaggggtgttgcgggggcaacatgagagcccctcacccccaactctccactttcaggaggcccccagtgaagagccccacctcggggtcacaataaagttgcctggtcaggaaaaaaaaaaaaaaaaaaa

SEQ ID NO: 9 - protein, 214 aa, Human
MNLSSASSTEEKAVTTVLWGCELSQERRTWTFRPQLEGKQSCRLLLHTICLGEKAKEEMHRVEILPPANQEDKKMQPVTIASLQASVLPMVSMVGVQLSPPVTFQLRAGSGPVFLSGQERYEASDLTWEEEEEEEGEEEEEEEEDDEDEDADISLEEQSPVKQVKRLVPQKQASVAKKKKLEKEEEEIRASVRDKSPVKKAKATARAKKPGFKK

SEQ ID NO: 10 - DNA, 19951 bp, Human
ggcatccccatatgatggttactagcggctgggaagtgggggtggggggaggatgaacagaggttgattaatgggtacaaacatactgtttgatggaaataagataagttgaaataagaaataagttgatagtacagtagggtgactatagttaacaataatttattgtatatttcaaaatagctagaagagaagatttgaaatgtttccaacacaaagaaatgataaatgtttagccgggcccagtggctcatgcctgtaatcccagcactttgggaggcctaggcaggaggatcactgaggtcaggagttcgagaccaacctggccaacatggtgaaaccccatctctagtaaaaatatgaaaattagctgggcatggtggtaagcacctataaacccagctacttgggaggctgaggcaggagaatcgcttgaacatgggaggcagaggttatagtgagctgagatggcaccaccgcactccagcctgggtgatgagagtgaaacgccatctccaaaaaaaaaaaaaaaaagaaaagaaataatgttgaaggtaccccagttaccctgatttgctcattacacattgtgtgcaagtataaaaatatcatatgtaccccataaatatgtacaattaatatgtatcagtttaaaaagttaatgacgatggtaccaatattatagcctcatttagcagatgaggaaactgaggcactgagctatgaattaacacacctgaaatcacagagcacagtccagacttgaacccagactgtccagttccagtgtcccagctctaggtcatgacctcagggtcatccccctcccctgcctccatttagccttcactgtgacccccagctgcagcctgacatcagtgtgattattcacggggtgggtagcctggggccactgaaggccggtttgctttgagcactgctctccaatgaggctggaagccctttgaggggctgtcgtattcaccgcgggatgcccaggtcccgcccaattggcggaatcaccgtttgttgagtgaattcttgaacgtctgtgcatggcatgcatgtgcctgccatttgctcatctttaccacacacttggctgcaatggctgtcaccttcaataggcgccttgccagggacagagagctcagggacagccaaggagactggaactgctcagctggggatagggagcctagggggccctggcaggcccccaagcctctcctgggttgtcctggccaattcacagggaggctgaccccagcttccaggaataaaagattctgacctctccgtggcaatgagcccctgtcccaggggtgtgcgggagatccctggttatctgaggtgtctccagggtatatccgctgaaaccccacttcctcttcactgcccactgagcctgggaccagctgctggtcacgtgcttggccctgtagcgtcgctagccgtgctcctcagttgtgctccccgccccctgccgcggcgcctcgcctcccggctcacctccccaccccacctgcccgctgcggctctccggcgggagatctcaccgttctggagacagggctcgctcgctctcacggtaggctggaagaacgggctgtctgggccttaggaaaggcccatgctgtataaggcatggggaaaggaaaggaagaaaggcaacgaacaagaaggagggcttccaactgcagcttcctgccggctgcaggcctcccttcctaagctgagctgaggcttcctctccatgggctggggagggggcgccaggaggcctcggcgggtccgcaattggccgggacagcttctcacgaaaggtcctgggccggcatcatcagcctcacctgggaactggttagaactacaaattccctcggccccacccagaccgacgccaagggcagctgtggagtggggcgcggcaatgcgccccttaacagccctccaggtgattctgccgcgcagaggaggaaagaatgggagaagggaaggggagagggaggcggcttctttgcgactaattggacacctgcccttccccttcccaggcttcttagcccgggcttggacagccgccttccggccagaggggatgaggttgcgctgcgctccgggagcgccgatggcgtgactggccccgcgcggagcagcgacagtaaggctgtgtggggggagctgggacctaagccgcgcgcacacccctttctctgcgtctggtggaggtgcacagaggcttttgagtcaggcccaagcgcagccaggtgacctccccgcggcctttcaagcctgagctcggtggacagctccctctcccgtgagtcccgctgtcctgtacgcgcccggtcgagccccgggctgcgcaccccgctaggaggtgggtactcgtcctccaggagttgccggtgagcccttgaccgtggcaggtcccctccagccgcgagcgacccctcagtacctgccgatgcctgctggtctctggcatcctccagtcgagggtcagggtcagggagcaaggcctcacgcgggcgccctccttgcagctgcccggccagcccgcttctctgcccggagccatgaatctcagtagcgccagtagcacggaggaaaaggcagtgacgaccgtgctctggggtgagtggggactcaggctccttcccagagacacgccccacctccggtgcgcggcagcttggggcgcaggtgagcccctcctttgggaacgaatggagggccccacttccctccctttctcctccgcaggctgcgagctcagtcaggagaggcggacttggaccttcagaccccagctggaggggaagcagagctgcaggctgttgcttcatacggtaggtgttcccaaaagaggggaggaagatggtgtccgggaactttctggtcccaacggagggctatggatttctcccgtcggccctcagggtgatgaggggcctctattttcaaccccgctcagatttgcttgggggagaaagccaaagaggagatgcatcgcgtggagatcctgcccccagcaaaccaggaggacaagaagatgcagccggtcaccattgcctcactccaggcctcagtcctccccatggtgcgcatttccctgctggctggaagactgctgtcagcctcaccctcacccttgggtggggatggacacacacgagggtgcattcaccctacagaaatgagccatgctagggaggtagcacagcccatgcagaaagccggggtccagcccagctcaccccttcctagagctgtgtgaccttgggccagttaagctgtctgcaaaaattacactttaaaccaggggtccccaactgccgggtcaaggaccagaactcgtcctgttaggaaccgggcagcacagcaggaggtgagcagccagctagccagcattacccctgagccctgcctcccgtcagatcagagcggcattagattctcagaggagcatgaaccctatggtaagctgtgcgtgcgaggggtctaggttgcatgcttcttatgagaatcgaatgcctgatctgaggtggagcagtttcatcccaaaaccaccccccactcccaccccatccatgcaaaaattgtcttccaggaaaccattccctggttccacaatgattggagactgctgctttaaaccattcactgctagtgacaggaacatggtcgatctacatattggttgtgcatccagaaattctctagtttctaataacttaagttttctatgcatataataattgtgaataatggcagttcttgccttttttttttttttttttgagatggagtttcactctgtcacccaggctggagtgcagtggtgcaatctcagctcactgcaatttctgcctcccaggttcaagcaattctagtgcctcagccttcggagtagctgggattacaggtgtgcaccaccatgcctggctaatttttgtatttttagtagagacagggtttcacaatgttgcccaggctagtctcgaactcctgacctcaggtgattcgcccgccttggcctcccgaagtgctgggattacaggcgtgagtcaccacgccaggtcttttttctttctttctttcttttaatcctactgcactggctaggccctgcagtggaatattgggtgaacatgagatcaggtctgacatccttatttccttcttgttttttaaaaaagaagcatttggtaagtacattttgtcaggttaaggaagtccccatctaaaaccctctgcttttaaaaaattgctttgctttgaaatcactagagggggttaaattttaccaaatgcttttctattcatatgattctaggttctttagtccacgtggtaaattgtatgaagagaataatatctgagttcctttgccatcctgggataaatactactggtcatgttacaaattctagatttgttttactcatatattgagcccttttttttacctgtatacatgtgaggttggcctataagtgatagtatttaggtgttcaggacatgctagcctcatgcactgagggtggagggttctctcctattccctggaacagtgaatggaagactaggatcagctgtccttggaggtttgggagacccctctgtcaaactgccctcagactttcctgttctatttacacatactttgcaggcgatctcatcctttcctgtggttttcaagaccatctaaacaaatgaagactcaggaacttatttctgtaaactcaacctctcttgagctccaagtcctatatccaactaaatggctttgatagatatctaataaatatcccaaatttaacatgtctaaatccacatattcaatttttatctctaatccacccgcctccccttcaacctgattttctctcagaaaacagctgtttttccagttagccaagacaaagctctttttttttttttttcccctgagatggagtttcactcttatcacccaggctggagtgcaatggtgcgctctcggctcactgcaacctccgcatcccatgttcgagcgattctcccatctcagcctcccaagtagctgggattacaggcatgttccactacgccaggctaatttttgtaattttagtagagatgggtttttgccatgttggtcaggctggtctcgaactcctgacctcaggtgatatacccgccttggcctcccaaagtgctggatttacaggcatgagccaccacacccagccaaatcctcttttttcccacacccatatctgatctaccagcagtcctgttgtctctgcccccatcttataccccaatggaccacatctcatcatcttccctgctacccctggtacaggtgacagttgcctgtggctccattttaattgcacagccttccacctggtctacctaccatcacatggtcccctgtagtctattccagggtaggcaaactagagggcttgaatctaggctgctgcctggttttgtaagtagttttactgggaacacagccacactcattcgtttgtaccctgtccatggctgcttttcctccctaacagcagatttgagtagtctccatggagaccatatggtttgcaaacctaaaatattaccttctggctcttaacagaaagtttcctggtctgtgctccacacagctgccaaaaagattttttttttttttttttttttttttgagacagaatcttgctttgataccagggctggaatgcagtggcttaatctcggctcactgcaacctccacctccttagtagccgggacaacaggcgctccccaccatgcccagctgattttttttttttttttgtaatttttagtagagacggggtttcaccatgttggccaggctggtcttgaacttctgacttcaagtgatccacctgcctcagcctcccaaagtgctgagattataggcgtgagccactatgcccagccaaaaagatccttttaaacacaggttagatcatgtggcttctctgctagaatagttaggtcatggctctctctcatttggaataagagccgagagtgtattatggcctgcttcgaagcctttgtgttctggcctcagcaacctctctgtttcaggtgtgttttatgtcttatgttccaggtatgtatcttttacacagtatgtagctagattttgttctatctggccagtatagtctgtattcattgtgattgctgatgtaattggatttgtggcatgtactttgtacccctactttccttgcttttttattttcttctccttttcctatattttattagattaattaaagttccttttccccttctctactggtttggaagttatagaatctcattctaatttttttactgtttatctttaatttttttaacaatcatacattactcaaagtttagaattaatgttttatgtcctcccagacaatccaaggagcttttctgattctcctttttttatttttttattttcgaaatggagtctcactttgtttcccaggctagagtgcagtggtgcaatcttgactcactgcaacctctgcttcccaggtgattcaagtgattctcctgcctcagcctcccaagtagctgggattacaggtatgcaccatcatgcccagctaatttttgtatttttagtagagacagggattcaccacgttggccaggctggtcttgaactcctgagctcaggtgatcctcctgccttggcctcccaaagtgctggaattataggtgtgagctaccgcacctggcctgattctcatctttttaacttcaatattattatcaagtgttctagctccaacttgtcagcctactcaacactactcattactagtactattgtattttccagttaattcttatgtaggtttactttgtttaccaattagtttggttaccactgcttctagcacccacttcttccttcttgatctaatttcttatcctttttagcaacatactttagtaagcctgagaatagtaaacttattcaggctttgtctggaaatatcatgattttgccctcattccttcatgatggtttggctgtgtatataattctagatagagagtttccttcagctttgaagctgttctattctggttcccaatgttgctgttgaagtcctcaatctgtctgattattccctttttggagatgtctttcctctctggcttattttaagataatgtcttttgtttttattttctatagttttaccatgatgtgttcaggtgtagatttattttttttgtctgttcaggacttaggttttcagacatgaggatctatgtctttgatcaattctggaaaattattggttgtttttcctttgaatattgactgtcttccagtctccctagtcccttttccaattagatatatgttgggccttctcactctgtcccccgtgttagctcccccaatgtttctttaaatctctccctctctgccttatactgggtaattccttcacagcattattgatcatactaattctgcttctgctggttttttctgctttttaatcatttgatcaggttttcttttatttttgttttaaagacaaggtcttgctctgttgcctaggttggagtgcagtggcctgatcatggctcactgcagcctcaaactcctgggctcaagtgatcctcccactcagcctcctaagtagctgggactacaggcatgagccaccatgcccagctaatgtttattatttttttgtagagctggagtctcactatgttatccaggctggtttctaatttctgacttcaagcactcctcccacctcagcctctcaaagtgctgggattataggtgtgagccaccatgcccatcctcagctttgggtgttttatatctagaaagctgcatttggttctttttcttttcataatgtcttgttcttatgattatgattcctacttttatatttacttggttttaaacatatacatatatatatatatgtacatatttctgtatatacatattttagagacaagatcttgctctgtcgcccagtctggagtgcattgttgcaatcatagctcactgcagccttgaactcctgagttcaagcgatcttccggcctcagcccccccgagtagcctgggctacaggcgtgcaccaacacacccagctttatctgatatttttttagatcagtgttctaagttcttggtcgggctagtctgcagtcacttgatttctttctgttgactctagctggattgtttcttgtatgttttgtaattttatacggtgagctcatctttagtttattttgtttttttccaaaagaattcgatgtggcctgagtttggggagtgttccaacagagtggtttcgtgtttgcttctgccatttacctcaggaatatcataagcttgggattttctgtacatttcttggcttggcagtactcactgagtaaattcagaccccataagtgagaggtacagctatggggtatgggctctcactggagacttcttttatttccattcatgctttgttgttagcttcctttaatggtggctagggttttgatttttgtttttcatttttgaggagaagagggctggtttgggcttttgagtctctaatcctcaacacttaccttgggcctctcactaagggtatagcccttgagggtcctaccctccccatggtggtctcagctacaactctcctccttgcctgagcccaaggccttgtctcctgactgtgaagattttgttgttgttctgttgtgttttttaaagacagtcttgttctgtcgcccaggctggcacgcagtggcgcaatctcggctcactgcagcctccgcttcctgggttcaagcagttctcctgcctcagccccctgagtagctgggattacaggtgtgcaccatcacacctggctaatttttgtatttttagtagaaacagggtttcaccatgttgcccaggctggtctcaaactcctgacctcaagttatccgcctgccttggcctcccaaagtgcttggattacaggcatgagccaccgtgcgtgctcggcctgtgtgtgagttttgaagcaaaaagtcctggctgttttcaggtccatttcccctcggttgcagcacaccagctcctgcacctgcctgtcttcatttcttttttctttttttcattcatcactaatcagaaggcatcctctcttcattttttatgtacgaggattcttctgtcttactgttcagccatgcagtagaaacactgaattacatcctctctggcatttctaagtgtctctggctgcagagtttgttttcacttattacctcctgtctgaacttagagtttagaagctgtaagttattcactctaagtctcagtttcctcctctgtaaagtatcagtacttacatcatgggtttcttgtgaggatttaatgagataaagcagataaaatgcttagcagggtgcctgacacgtggcagaagctcaaaacaataagctatcattgtcattcgagaaaatttgggaagtttggaaaagtataaatacaataaataccttactatcgactgacaattatggttagcactttaatatattttaaacctttattcttatgtatatccatacattatacataagacaaaagtagtactgtagaggctcttgtcatttataagtgtgatgattggggtttcacgctcgtgtgtaaggtgtgcctcccacaaacctggttacgagttggcacatcacctgtctgatgtgaagaaagcaagcagcactgtacataaaatcatgcatctgctttctcgtttgatcggtgtctcagtctgcccaagttgctataacaaaataccacagactggagggctttagcaacagacgttttctcacacttctggaggctttgaggtctgagatcagggggccggcatggttgggttctggtaagggtcctctccctgagttgcagacggcagccttcttgctatgtgttcacgtgtggggcaggagagtggagagaaagagagtgagttctctagtgtctctttttgtaaaggtactagttctatcatgagggtcctgccctcatgaccccaaacctaatgacctcccaaggcctgcatttctaaatactatcacactggaggatagggcttcaacataggaatttggaggatggggggaatggccataatttagcccatactaatcagattcctctatctgacggctgcccttcagcttggaagcccatctgtgagcttagcaagaatgggattaccaggttcaaattcttggacagaatccacctcagcaggggctgagtagtgtgtagggtttggggagggaaatcaactcagtatttctgcctgaggccagtccccgagaggtgccaggcctaagtggccctgtcttctctgcttcctccccctgcaggtctccatggtaggagtgcagctttctcccccagttactttccagctccgggctggctcaggacccgtgttcctcagtggccaggaacgttatggtaagtcagagcctgcgatcaggaaggtccgtgagtaccgtgctaggcaggggctcgggacatactagctactcaaacactggagggattcttgaatgttggaagaaaatccccaaaggcaacatgacagccagcagcctggactggaaggcaagggcgctggtccctgttcttcttctttacctgccatgacctctgtaggctgcagcccctcacttgtaaacttcaaagagcagttgtgaagaataaatgggatattcaggaaaagcactcagcgtaatacccagcactagggaaccactgttcaggatgtggctgctgcagtgatgcagaccatagcaacgcagaccatagcaacgcagaccatagcaacgcggcatgatgctgactccttcaaggtcccttcaactggccctcttttctgtatgattatgcctcattcatcagggtactctcctgctaaaaagccttggcaggtcccacttctcttaggataaggtttcaattctttagctatttgttttagattctttccccctctcctcctcttcctcccctactctgcctacctttagccttggccccagcccttgccaatatgaatccccctcctacccagccagagccacttcccctgccctctttctaccaccccagccctctgcagggctttcctagaccccctaccctaccctggcctccactgttgggagggccagaaagggtgccgccctgtacaggtggcaggcaggtaaccactgtcaactccaggctaggattcctccagggcagtgcttggagcaacacggatcacagaatgggaggtgggcattgattctgtagctctgaagctgtgcccctgcatcctttcccatgctattacagaagcatcagacctaacctgggaggaggaggaggaagagaaggggaggaggaggaagaggaagaggaagatgatgaggatgaggatgcagatatatctctggaggagcaaagccctgtcaaacaagtcaaaaggctggtgccccagaagcaggcgagcgtggctaaggtgggggaaggagcgtggctgtttggaaggaagtggtacccctacagaagcacttaagaggggtgggccaccgggagcctgggccagcctcccagaatgagtgtacaggatgggccaaggccacctcagctagttctggccaggagctcagcagggaccttgtggactttgggaatctgttgtggctctggactttgtctgaactctcataatacactgttttttggttcccagaaaaaaaagctggaaaaagaagaagaggaaataaggtaactctttctacctattaaattagccaaagtctccagctgagatatacagtgttagaaagaatactgtgctgttgggatgtacgtgtacaaatgtacacacggtgtgtctacctgcactcgcaggcacatgggtatggaagtgctgaagggtggcatcacctttctggaagagcattacaacgttcttatcttgggatctaattccagtgaaggcaattccttccacagaattccatccaaatttcaggggaaattacctgcactaagatgcttctcacggccaggcttggtggcccacgcctgtaatcccagcactttgggaggctaaggcaggcagatcacttgaggtcaggagctcgagactagcctggtcaacatgatgaaaccctgtctctactaaaaacacaaaaattaaccgagcctggtggcattcttgtaatcccagctactcaggaggctgaggcatgagaattgcttgagcccggggaaaagttgcagtgagccgagatcgtgccactgcgctccagcctggatgacagagcgagactcagtctgaaaaaacaaaattttaaaaagacgcttatggcattattcgaatagtgaaaaaatggaagcattctaaatgtctaccaatataacaattcactaagctacatccctcttctcaatggaatattacataatgcttatgaagaatatagcaacctggaaagtatgtgtatagttttggtttgtttgtttaatgagacagggttttgctctgccacccaggctggagtgtgatggcacaatcatggctcactgcagccttagcttcctgggctcaagcaatcctcccacctcagctttccaagtagctaggactataggcacgtgccactatgcatggctaacttttaagttttgtgtagagacagggtctttctatgttgcccaggctgatctcaaactcctgacctcaagcaatcctcctgcctcagcctcccaaagcactggaattacaagtgtgagcctctgcacctgataagaatattgatagttcatacagcaggacataaagtcatttttattttatttcacatatttttaaaaagagtttgaccaggccgggtacggtggctcacgcctgtaatcccagcactttgggaagccaaggtgggaggatcacttgaggtcaggagttcaagaccagccttgccaacatagcaaaaccctgtctctactaaaatacaaaattcagctgggcgtggtggcatgtgcctgtaatcccagctactcgggaggctgaggcaggagaatcacttagacccggaatgcgaaggatgcagtgaaccaagatcacaccactgcacaccagcctgggcaacagagcaagactccatctcaaaaaaaaaaaaaaaagtttgaccaagaaaaaaataataaccctgaaagaaaatacaccaaaatgtttagtgtgggcagtaaagaaaactataagtaatgtattttcttgtctatttgctatattttgtacaaaatggttaatattttataatgaaaaagacatttgtgggccaggtgtggtggtgcacacctgcagtcccagcttctcaggaggctgaggcaggaggatcactcgagcccaggaggtcgaggctgcagtgagctgtgatagtgtcactgcactccagcctgggcaacagaaccagactccatctcaaaaacaaaacaaaacaaaagacatttgtgataactaaatgaagatggaagcctaaggaaaacacatatgcgtatgcatgcacacgcacacacatccctttgtttaaagagtccgagtggtccccaggaggagcagccaggcttgctttccagggtgggcactgggagggccacgccgctggtctggagctgagctctctccctgaccccaatcccactcctgctccgctccaccctgttgcagagccagcgttagagacaagagccctgtgaaaaaggtgagtaggaccagagggctttggcccttgggacaggcgagtattctctggagggggctgcctggtatggagaagggaacgggaccctggagccctgccttccctccacaggccaaagccacagccagagccaagaagccaggattcaagaaatgaggagccacgccttggggggcacggtgcaaagtgggccttccctgggctgtgctgcaggcacagggtgcccctgtccagcccctccacctgtgtctgaatgcaacaggggtgttgcgggggcaacatgagagcccctcacccccaactctccactttcaggaggcccccagtgaagagccccacctcggggtcacaataaagttgcctggtcaggactttccttctcttccctggagccagcctccttgtccgctgcaccagccccagtgcccggcagagggcagccttgaaccggtgcacccgggccctgaggtcatacctgcctccctgcacccagcccccgcggcctgagcctgctgtgtccctcgtcctcggcacccccaattctcccccagtggcgagggaagagcctagagtctgccttctgctgagctgtgtgtcaggtggatttccagcctgcaccctccctctgggcagagctaggtttataggcacccaagggctacggctgctcaagctaccagaaggggcctcgccctagggggccagcccccagggtcttctcctgaccttattcctgtcaggcagctactgtgtgcagagcatctatagggaactcagggacatccgctctccctgcttgcttccgttaggggccagctcatcttatagggacctcccacatgtgaagatctgtgtcaggcaggagccagaggcccgcaccttcaaaaaaacctttgaggtggagtagacagggtgagctttacagaggcgccaagccccacatgctatcgagtaggcctcagtcaagcataggggcgaggccaagagaggactggaaaatggggtgggggacccccaccctctccctggtgtgcagaggggactctggagggctgttcacctgtgggtgaccctggcgcagttcctagaacaggcggacacacagatgagccctacactctgggttatccatgcagcgcctctgctggcttctccctgcccctccccagcaccctctggggtcaggcccgaagtgaaccagtggggagctggtcctgctgtcctgcttagtacccccaggtatggggcccaggaggtcggagctctttgaacacctgcctaggaaagtcaacaaccaggctggggcctcctgtccagctatagcttcttttgagaccagagacagaggtagcagagggcagggttgtattcattttttttttaatttttattttttttagagacagggtctcactctgtcaccgaggctggagggcagtggcacagtcttagctcacggcagcctcgacctcctggactcaagcaatcctcccacctcaacctcccaaagtgctggaactacaggcacgagccaccacaccaaaacaaattttaaaattttttgtagagatggggggtcttgttaagttgcccaggccagtctccaactcctgggctcaagagatcctcctgcctcagcctcccaaaatgctgggattacagacgtcagccactgcacccacccagggtggcgttctccctgcatgtttcctacacccatgagacagatgtgggtgctgtcctgccctccaacagacaagccactaactttaggtcacccagagtcccaccctccaacaaagggacaaaccactaacttcaggtcacccagagagtgacaagggggactgctccatgtgagcagctggtgctttttgaacttggtttcatctacagtgacccggggtaacccaattcctcaccttcaagtcacttacagtctagtggaaacaaaacccaacacaatttcagttactgtctggtgctttgaagggagtggaacaggtgaacttgaggggcaggaggaagcagtttggtgagaaggtctcctggaggaggcgacggacaagaagggctcaatgggcttcattaggagctggcagaggacgttcctgggaacaggaacagagcatgcaaagggtccgaggcagagccccacttgaagggggatcggctgcagtgacagcttctaatacccacgacccactccttcaaccctcataaccgtgccttaaggggattgtgactggctccatttctttctctttttaattttttttttagatactgggtcacactctgtcagccaggttggagtgtagtggcacgatcatggcttactgcagcctcgaactctgggctccgcctatcctcctgcttcagccccctcaagtagctgggactacaggcatgcaccactatacccagctcatttttttttaacatttttgtagagatggggatctcactatgtggcccaggctagtctcaaactcctgacctcacactatccttctatcggcctcccaaagtgctgggattacaggtgtgagccaccacacctggcccagccccatttcccagatgaggaaagtgtggcacagagaggttagacaagttgccccaaggtgacacggctggcagaggagccagggagtcccaccccagagccctggattttgaccactctgctgatgggagggaggcatgagccggtgcacagtttatgaagtcgtgtaaactgagagcaggagttagaagtcagtcaaccatgtaatgggagtcctcaagggacagctaggcgtttctacagccaagcgcatatttggccccaagcacaagaaggcgcgcaacagataaaccagtgatcacttgattttgatttgcaagcaggcagtaggaaataaattgcaaaggtggaggccggatgcagtgtctcatgcctataatcccagcactttgggaggccaaggtgggcagatcacttgaggtcgggagtttgagaccagcctgaccaacacggagaaaccccgtctctactaaaaatacaaaattagctgggtgtggtggcgggcacctgtaatcccagctacttgggaggctaaggcatgagaatcatttgaacccaggaagcagaggttgcggtgagctgagatcgcgccattgcactccagcctgaacaacaagaatgaaactccgtctcaaaaaagaaaaatttgcaaaggtgagggtccatcctcattgctagggtgccctttgccctctgccctttgcccttcccctgccccaactcttctgtttttcagcaggaagagggtgggctgggctcaagcaggtggtggcaagtggctgacctgcaggtgggctctgtgtttgcaccagctgggctgttaggagaggcaggcgtgagacgaccccagctggggggtgttgaacttggcactatggggtgaggattaaccacagcagcccaggctacttctcagttcccttatcacctcctgaaccccacccccccagcaatgaatgttaataaaccccacccttcttccctccccctttccccgagctcactccagtcaagggagagagtctgacagtttaggtcaactgaggctaagccacaaaaagggcccctgcccccattcttgtggcacttgatgcgtttctgtgagtcctttatctcagctgacgtggatggcggtggttttgacagtatccctgctggtagccatttcctttttataaactgggaccctgaaaccagagaagtgaagggacttgccacaggtcacacagcgtataaggaccagggcaaaggggcggggataaaatcaagggctccatgctgcctccccacttggggcccaccactggcttccccatgggcgtaaaggagcaaaccaagttagaaggccaggcctgcaggtgcccaacaggaagggacaaggagccacagctgtcttgcctgtgacacaggccatccagccatgcccagagctaaccccctggctaagccccgaggcccagcttgactgctggcatctgttaccatggagacccaggctggcctgagggctgggccagtgatggcaggccctgtccccatggatagaaacaggtgcttgggctcagaggccttgagtggctcccactgtccccatggccagtgagtcccgacagcataaattggaaccgttacccacctttcgccccccagctgacacctccaccaacccaaggcctgagctgtcccctccacgtgtctgtgctctctttaatgccctgcctgggggctgggagtggtgaggatgtggatgtgaggttgaaggtttctcagggaatgagccagagctgccagaagaggcagagtgtaacccagactgcagatgatgggaagaacgcggaacagaagtgacctgaaggatccgcagggggaaagcagagaggtgggcacgcgggcacctggtaccttgtcccagccatgccaccagctagctgtgaggctttgggcgagtcccttgccctctctggctctcatccaaggaatgaggaagttggaacaaatgatgaatccctaagaccccttctaggtttgacattctttgagttgcattccaaaaccctggactcccccaggtaagcaaggccagggcttgccccatcctccccacacaagctcaggcagcacccactcctgggctgggttcccgaggaagagcctgcggagaggagaccccggagctgcctgcactggtcagtgcatgggggcaggggtggcagaccactttgtggattgatggagctcaggaaggtgagaagggacccacaggtgagagtttcgctcccctggtcatctctttaggtaaataaatccacatccgccacttccccttcccttcccaccctgggggcgctgagaactccagggagcccagagctgaggcctgagctctgcttgctcacactgggtcttccctcagagacccccaagccctcctatcttctgcagtcaccgtcatccacttttctgtagggagggaacagcatggagctctctgttcaccggtctccaggacctcggattccacctttaatcctgaaaacccaggaaggcttctgtatccctacaatgaagcaggtttggggctggatctgcagggtggcaactcaatccatgcagaacagaagaagcatggacttttccattctggctattccattcactagctgggccattctgagcaaactacctcccaatgtgcctcagtttcctcatctgcaaaatgggctagtcgctggcattgtaccgagcagtatgagaccggaggtcatgggaagaggctggtaggcacttcatcccaggcagctgctcagggacatgggacacagggaggggactccgagctgctcctagctcagagaggctctagggacaggcactggaaggaaggggatgcagaatggtgagtggagctgggtctcagaacacagacatcttgaagtctgctatgtgctgatttcacacttgaccccccaacaccctgaggcattactgtcctcatttctcagatggaactacagaggcccaaggaaaaggggcttaggccaggacacccagctcttctccaagtgtctgtctcaggctagcttttgcaactctccattgggattcttcctagatctccatctgtacctgccaacccacctctgacccccaacctgttgcgcccagtatttgctgccgatcaggacagccttaacccctccttcccgggcaatcagctgctccaagccaagcccacccctgccccctggagggaggcggctccttttaaggctgcttctgggaatttccactccagagccagaaccagagaccccagccccacttgacacctgcggctcacccttggggaggtggggcgccagacctgagtgcaggagacgcagacctggaagggctccccctccctgacctgccacacatcgagtttgtctgcgtcgagtttggccagtctgtgagggtcaggaatagagcaggagacagcagggccacctccttcagaaggcccccaccgctccatccctgc

SEQ ID NO: 11 - DNA, 6873 bp, mus musculus
ggatccctgttgcagtcataccctatgggaaaagagcaacttacctatcttaaggagattgggggaatttagatatttgtgcatcctctttcacttgaatgagaacaatgtaccagatcgtcaacagtgcacatttgacccggccagtagcaacatacctaaaactacctcaccattgtaaaacaccctaagaatcacaagaaacttacagtttttcagagacaaatcaaggagcagagttacagaattaaaaacaattcatcataccttagaattttctctaatcgaatgagttgataattgtttccatactcttagccatctttggagctatactttgaaggttaaaaatatcctagactaagttagttttcattgtaagtgctagcagcctttgctttctggtgtgaaataagaactaatttcaagtagaaagcacagagttcaggagagatgaagactatgttgcccaggctagtcctcagcccataactgggttcttgggctaaattctagctgctccattggaaataagcaagacagtgaattaagcacacacgaagagtactaaacgttgcgtggccaaggccgagaggctgagggtctgcctagtaacggtaacctttccctgatctctggtttatcgttttgaagaccttttcagaaggagtagtctgtctgtatgtccttccatcccaagcaagtgaagagcccagaggagccactggatatcaagctaggtctccaagcagttatgtctaaggagcttcaggctttgtttgcgaggttagaatggatgtagcttgtccagatgcctcctcagtgatgttgttgttgataggttgcccatgtgtttcttttcttaccaggtttccctgtggtctctgcttgtgaccctggtggttcttttcataatgactggcaccatgctgggacctgaactgctggcaagcattcccacaactgtttacgtggtcgccatttttatgcctctggcggctacgcctcgggttatggcttagctaccctcttcacctccgcccaactgcaagaggactgtgtgtctggaaacaggaagtcagaacgtgcagcttctgcactgcgattcttaaacttcccgcctcgctttataggtagcatgtacatgttcccttctgctctacgccttcttccagtctgccgaggcaggggtcttcgtgttgatctacaaaatgtacggaagtgagatactgcacaagcgagaggccctagacgaagacgaagacaccgatatttcttataagaaactgaaagaagaggaaatggcagacacctcctatggcaccgtggggacgcatgacttagtgatgatggagaccacccagaccgccctctgactgaggagatacacgggagctgaaacatcacttcctatttgtgaccattggtagcgagtatggttcgcatccgggataaagatgggttgacatttcctgtaacagatttgctcttcccactgtaatgtagtatctcagtattacccatgtgtttttctaactcaacagagtgtcccaatattgcttacacctggatcagctaaagtgccgcgtcctctgcttaagtagtgtgctgtttgtttgtttgttttttgttttgttttgtttttttccatttccaccagcattgctacagataggaaatggggttggaaatgtttgtaaaacagaaccatgggtttgttcaacttacaaacaaccgattctgttcagggcgagcctgtattgagaaaagtccaaaacgggtcaaaaagggttgaaacgacaggatagcattgcatcgtcaagccagagaaaaccgtattaatgtgtgtgactacttgatctagtatctattgttaatggccatcaacattgtgcaggggtgaaaggcatttttccccatatgtttcctgtatgtgtataaacgcatctcagctccatttatcgtctgaaggaatgatttacttaggaaaatgcgtagacctcacctcagggagagaaaatgggccactttgttcatccgtgggaaagggctgtggctacaggctttccttccggaaaggcctgtggctggacactgtcccactgctctggtagactggagctgtgatctgagacaacctaagaggttcagagcagtctcctaaccttggtattttgctccctaatcagacacactggcctcccttgtcttcttcatgacagacatctggagctacagacatgggggcccacctggctcggctaatctcggtgatgattctggggttgaattctcatctcatctagttcccctacaaatccttgctgtggctagcaaggaaagctctttttctgcatccacgagggagtgggggtgggggtcgcctcttaaccagtgtggggaaggttttgctcctcatggcaacagcaggtggtagggctttttctaccagtgcgcggccgcctatttaacgcagcgtggagggcagctgggctgcgctgatggctgcctgggcgggcgaggcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgacagccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccgatcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgcgaggaaagcggtcccccagccgcgaagcgaggagggcgatgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctcgagaccaggcctccccgcaaagcacggagcaggacaaggagcgcctgcgtttccaggtgaggccagcctgatggcctggacgcctccagaattgtagggctccttcagggctaagctggtggctctgggtgatgcagaacatagaattcttccatgccatccgtctggttttgtttgtttgtttgtttgtaacatgtttggtgttttgattgcatgttgtatctgtacacttcgttgtagtggagagatgggagcagaagagggtgtcggatccggatcccctgggactggcgttttacagatggttgtgagtcaccatgtgagttttaggatcggaattacggtcctctagaagaacagggtgttgtttcacagctgagccatctctccagctctttggcatataggattttgcagccgctgcctgttaatacaatgggaggcgtttacacaataaaaaccaacccatatgtgtcctgacccactggcagcctctgctcctggggaatgccagttgtaattattctgatcacataaacgctacacatgaggtctccgcggagaatgcgcacagtctgggtttggaccaaacttcagatggctgaaggaagataagtgcacacatggcagaaacataatcttttgaacttcgttgcggggagagtcggtttcccaaggctcctttttttatttcccctctagatgatctgtcttggttaacttgccggcttgttctataccagccccttcccttcgtttctgaagctgtcaactgaagcttctctctcccaaacttgcctggcttaaaaaacaaacaaacaaaaacaaaacaccccccccaaaaaaaaaaacaacaaaaaaaaaaaagaaaagaaaaagaaaaagaaataaaagaaaaaaaaaaccactctccccattcatcgaggccagccactgctaagctgttggatggtcttgagttgctgcctgtgctagcaaacaaggaggcacaaagagtgctgtaggtcgtatacccccaccaaagaaatggagagccctgagctccaggagaggactctgagacattccttgtttttcagtcatttcaaggctggtgtgtttgaggttggggtggcagtggaatggggtgtcagaaaaaatagaaaagtgcttggcggttgctgttcacagctgggtgtgatctcttaggcagaaatcccaagttttcgggcctctgtggtggtcgttcacctataaaaaattgcattaagagttcttccaagccctgccactcctaaagacttagttataaaaacttgtttccaacttgtttgtcactaagtgggaagcttgggaagtttaagaaccaggtgctaacactatgtagttcataccaaatgagctagacttgggtaggtagcgggactcttttggaaacttacctagcatcaaggaaaatttagtattggttgaagactttcaaaggttttagaagagcctttctctttcggcataacaactttcccatgtgtgagtgtcctaatgcatcgcccacataaaatgccacgggaagaatcccaaactctaaaccgcacgatttggcttctcccttgtctgaggggggaaaaaccacttatcggtctgctgctatatgaactatcttgtttggcctccgtttacatatttgtttgattgagctattagttcacctggttaacttagaggttgacccaagtctaaccttactaccacggtaatcttaaagtatcaagtggaatgtggtcccaggttctgaaaattagggtcactcgggcatcacttgcttaaagtctggtaccctgctgttcagttcttagagcagaagtacggctactatcactgcaaggactgcaaaatccggtgggagagcgcctatgtgtggtgtgtgcagggcaccagtaaggtaagagacaccgtgcagccctcctgctctgctgtgttgccgagtgtctgctccatgccgatgtctttctcctcgcaggtgtacttcaaacagttctgccgagtgtgtgagaaatcctacaacccttacagagtggaggacatcacctgtcaagtaaaccaaacgtttgcattttggaagaggggtttggtgcacgactttgagtatatttcctgaaggaggtggtttccagtagctttaggctctaccttttccctcctccttccttttcatttttgactaggttggtggtagaaagtcccctccactgtaaatggggtgtttactcccttctgctgttgtaaaacttgattgcatgccctctcttgcatctggttaccttgttagcagtagaaagggcttgcttacctggcttcttcccactcggacctaagggaaaacatattgcaaaacagagtgcctttctgctagcttgagatggtacacattaccccaatgctacataggaaacacattcccaagttagcatatgaaacacaagaaattgagctctggcttttcttgagagtttacaaagggagtttcctgtaagaccatcctacactgtctagctctatgcagtttacccataactgtggctaagagtttgcttgcttagtattaatttagcactgtgccaagggacttagataaccttgaaaacatttacctgttaaaattaatgacagagataaaggaattcgaattccacatctgagagcccagtgcacttaaagttggtaattggagaattaattaccttagggtgggccctgtgaaaccgagaatggaaagccactaaagactccatctagaaaaggggactgtagtcacttttctacaataaggggccttaaacttccctaagcttccctgcacttggttctcagtgcccagcacacaggccacttgttctgtaatctgttttgaagctccaagaatcgagtggagacagggctcaccctttgtacttttcactccgatttttcagaagttgtaaaagaactaagatgtgcctgcccagtcagacttcgccacgtggaccctaaacgcccccatcggcaagacttgtgtgggagatgcaaggacaaacgcctgtcctgcgacagcaccttcagcttcaaatacatcatttagtgagagtcgaaaacgtttctgctagatggggctaatggaatggacaagtgagctttctcccctcttcacctcttccctttccaaattcttcatgacagacagtgtacttggatataaagcctgtgaataaaaggtattgcaaacaagtttgaggctttatccaattcatgtgtcagtttgaggggtgcatgtgcggagagtcaataactttcttaacatttgttgatgagagtgagtcaggctgacttaaggaagttaaaggcacctcattcaacaattaagatttttctttctttttgtttagttttattttatttataaatatatgagtacactgtagctgtcttcagacacaccaaaagaaggcatcagatcccattacagatagttgtgagccaccatgtggttgctgggacttgaactccggacctctggaagagcagttggtaaacccctttcttaactgctgaaccatctctccagcccaaatcttaaggttttacagacaagaatattacagg

SEQ ID NO: 12 - DNA, 4090 bp, MOUSE PSOOL
  misc_feature (1)..(4090): n equals unknown
ggcgggcgaggcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgacagccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccggtcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgtgaggaaagcggtcccccagccgcgaagcgaggagggcgacgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctccccgcaaagcaccaagcaggacaaggagctcctgcgtttccaggtgaggccagcctggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaccctgctgttcagttcttagagcagaagtacggctactatcactgcaaggactgcaaaatccggtgggagagcgcctatgtgtggtgtgtgcagggcaccagtaaggtaagagacaccgtgnnnnnnnnnnnnnnnnnnnnnnnnnnnnnnnnnnnnnnnnnnnnnnnnnnnnnnnnnnnnnnnnnnnnnnnnnnnnnntctttctcctcgtaggtgtacttcaaacagttctgccgagtgtgtgagaaatcctacaacccttacagagtggaggacgtcacctgtcaagtaaaccaaacgtttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctctgagttttcagagttgtaaaggaactagatgtgcctgcccagtcagacctcgccacgtgtaccttagacgcccccatcagcaagacttgtgtgagagatgcaaggacaaacgcctgtcctgcgacagcaccgtcagcttcaaatacatgatttagtgagagtcgaaaacgtttctgctagatggggctaatggaatggacaagtgagctttctcccctcttcacctcttccctttccaaattcttcatgacagacagtgttacttggatataaagcctgtgaataaaaggtattgcaaaca

SEQ ID NO: 13 - DNA, 7405 bp, Human
tagatgaagatgaagatacagatatttcttataaaaaactaaaagaagaggaaatggcagacacttcctatggcacagtgaaagcagaaaatataataatgatggaaaccgctcagacttctctctaaatgtggagatacacaggagcttctatcttgctgaaatattgcttcatatttatagcctgtggtagtgcacatggttaacataaaagataacactggttcacatcatacatgtaacaattctgatctttttaaggttcactggtgtattaaccaaacgttgtcacaaattacaaatcaatgctgtaatataatttgcacctggaatggctaacgtgaagcctgaattaaatgtggtttttagtttttaccatcaccaatttctatgactgttgcaaatacagaatctattagaaaacagggtcttggaaatgtagaattttggcgcactatgaggaaaaacaagctatctttgtaaagcataattgagtttaatgtaattgttgtaaaaaaaaaagtgtgcttgctctacttaaaattcctcacaatgttgaattttgacctgtattcagaagaattccaaaacaggtcagttaaataaggaaatatagtatttgtcaaaccagtatcagagaaaagttacattaatgtatttgattacttgatctggtatctacttattaatgaataatcaacatttttctagtggacaagccatgttcttttcgactggtcttctgcatttatgtaaacacaccccaaatcaatttatcttttgtaggaatgatttggttgggaaatttttcagaaccgctctggagcagaacaaaacggtacctcccggtcaccactgggactgggggaagggacacgctccaggggagaaaacaattcgcctagatgaagatccctgggtggttctccatgcactcgccgagggggctcagtgggtagcctcttagagcagctctgagaataaacttatcatctgagttaacgggtaatagacccagaacagttcccaaaccttggcactttcgctcacttagccagaggcacccggcctggcctcggcttcccggtgagggagcgggggtgggggggatgcgcaggcacctggaacaatcagggcaccgggagaagccggctcagctgatgccggtgatgagtttctctcattgaaatcctcctcacctcgggcgccttggttcccttacggatcagccctttcatcacaaagaaagccctctttccagatcatctaagggtcattgtgccaacatccgggcgtggagagtttcygtagggagaaggacgaagaggggccccctcggcggggacgcgggccggtggcwggaagggcgtggagggcggtgcagcgtgcgagcccccgccgagggccatccccgcctccgctcggccgcccgggcaagtcgcctatttagggtgcggcggcgggcgggagcagtgcgcccatggcggccctgggggacgaggtgctggacggttacgtgttcccggcgtgccccccctgctcgtaccggtacccataccccgcggccaccaagggcaagggcgcggcgggcggcagctggcagcascgcggcaggggctgccttcccgcctcctccccctgctcggcgggcgcggcctcgttgtccttcccgggctgcgggcggctgacggccgccgagtacttcgacagctaccagcgggagcggctcatggctctcctggcgcaggtggggccgggtctcgggccgcgcgcccgcagggccggcagctgcgacgtggcggtgcaggtgagcccgcgcatcgacgccgcggtacagtgctcgctggggaggcgcacgctgcagcgccgggcccgcgaccccgagtccccggccggccccggggccgagggcaccacgggtggcggctctttctcccagcagccatcccgtcgaggcctggagcagggcagcccccagaacggcgccccgcggcccatgcgcttcccgcgcaccgtcgccgtgtactcgcccctggccttgcgccgtctcaccgccttcctggaggggcccgggcccgcggcgggcgagcagaggtccggggcgtcggacggagagagggggccgccgcccgcgcggcttcaaggcccagaggagggggaggtgtggacgaagaaggcgccccggcggccgcagtccgacgacgacggcgaggcccaggccgcagtccgagcgagctgggagcagccggccgacggtcccgagctgccgccgcgagaggcccaggagggcgaggcggctccgcggtcggcgctaaggagcccggggcaacctccgtcggcggggagggcccgagacggcggcgacggacgggaggcggccgtcgcgggagaggggccgtcgccacggagcccggagctgggcaaggagcggctgcgcttccaggtaaagcctagggcggtcagggcacaggggagcccgggggtgcgggtgtcttccttgggcctggccctgtgactgcttcgggcactcggaggtgcggcgcttccctaagcgtgggcyacttccgtatttccgagacagccaatgaccgcgataggtgtcttccttgacagcacagtctcatgtccccgacatccagacttactcgtggcggctgctccacgggctggccagggcgacgcccttgggacgttcttataacccacatatttgcactgtaaacctcgcgcagtgggcgcataggccagccctgaccgcacggttggattacctatcagtaggcacaactgaacttcggagcacttgccggctggagagtcgattcccaaggatccctctctcccatttccgcactggatgtggcaaaaacccttcaactgctgggattctggtcagcaattctgatttctcctttacgagcttccaggctattggaaagctggagctcctaaaatgccccttcctaggaatttgctttgcttttaagaagcacccccaactcagaaatccaatactgcgaaagcatttggactgctcagtgttgctgcccgagggcagcaggctgaaactttaaagggctggggcacgcagagggcagttgtgacctagcagaagtggaaaggcacaagaggtggtaagaagcccgagggagtccctcgcggtctcttccacggccaccacaggctggtattccttttgaggggcggggttggtggcggtaggctgattgtgcgaggagtgaatcgagaggccagggcttcccagcgtggctgtgcaggagctgtgtgtgatcttaggccagtcataaccttcctggacttagcgcagtctcacaggtgagcagactgaattaagtgctccccagagttccttccaattctgaaaggctaactctaaaaacgtgtgcataactgcttgcttactgggagggaagaagggaagtttaagtaacactacttttgttcatattgaatatgaattatggcttacgtacgatttaggttcctggcaccactgtttgggagttaactagcagcatgaagaatgtgatcttgggtgaacctttaaagttccttagatgtggagtcttatttttcttcagcttaatacatgtgtgcatagtctaagatcaggctttatcttaaaaggccttcctacagaatcccaaactttagagaactgtttattatgtccctacttattcgtttattagccagccttatgaactgagttaatctggtatatgaactctaaggcccatgcttgcttaattgtttgactaggctattaagctcacttaattactgtattgaagaggcgtacccaaacctgacctgctttctttcatgatctgaagttgccaatttcaaatatcaagtagatccttcccaacgtctgaaatgaaggataattcaggtgttttgttggttaaattgacatatcctgttgttcagttcttagagcagaaatatggctattaccactgcaaggactgcaacatccgctgggagagtgcttatgtgtggtgtgtacagggaactaacaaggtaagaaataccaggtaactggcatcttcttgctgaaagtgtcaaggcgattttaagtttatcctctttgtcatcacaggtttacttcaaacagttttgcagaacttgtcagaagtcttataacccttaccgagtggaggatatcacctgtcaagtaaatcagatgttttgcattttgtctgacctgggcagtcgtcgagggtttttagtatagtttgagtatacttccaaaaagaggccaggcccccagaccttaggtttcaactggcttttgttaggagtggtagaaacaatactcagctgggaaacggggccttggtgttagcttctttctggccttgcaaatcttgctgttgttaacctcttctaaaactgttaacctcacttgcaatatggaagaatacttgtcttacttgctacttagtctaatgtataagaaaatcaacaaaaacatgcttgtcagctaacatgaggtagtcaaggttgactgttttaccgaaacgcttcttatgaagcacaaccttaaagtacttaagcacagggggttagtttgtcttgcctgaaagctcacaaagggacagtttaagataaatctaagttgtctagctttatggggagttgactataatggtaagcaagcaatatgttaactaagcattgcttaagcgcttgcttgctattaactgtgctaaggggcttagctaatctttaagaggaaagaagtgactacattcgcctcttgtcacacagctaatggagtctgaattgccagttgagacagcctaatcaatacacttgacccacgttggatatttaaaagcattaacaccctggggtggtggagagaaactaagtatggaaagccacttagaatcacttagatcagagctgggcatgtttctaaaagaggatgccttaaccactctgctcttggtgttcattgtcaaattcatccctgacttgttctctaccctttctcttaaacagttgttgtaaaagaaatttcacaattcataattggatctgatgcaatatagcagcagtacagcatggttaaacacccactattcctagccctgtcattgctacgtaggtagggatgtagagggaaaacaagattactatgggaccttgcttagagcacattcattaagtacttgaatggactagaaaaatgttgaagtcctaggaaatcactaagggtttatcttctgcatgcccttctgtatttttttcccccagagttgtaaacaaacgagatgttcctgcccagtaaaacttcgccacgtggaccctaaacggccccaccgtcaagatttgtgcggtagatgcaaaggcaaacgcctgtcctgtgacagcactttcagcttcaaatacatcatttaggtgaaagtcagtgttgctgtgcatgcgctgatggagtagacgagtgagcttttccgtgcctctcctccacctctcccttctcaaaatacttcatgaaaggcagtgtattctgaaaaagccttcaaataaaggtattgcaacacgatttatacattgcataaaatctgtctttgaaaataaagtttcaagagcgcttgtcttgtgctaacagtctgggcctgtcacttcacctttatgaatgcttgctgatggcatagagtgggccaggctctgagttaggctgcagccacttggaaaacaatttaggggggtgcttgtagacgaggtctacttatttaggcaggtctggaggactgaagcttagaaggaagttaactgaataaaaagccgcctagcgatcgcgccactgcactccagcctgggtgacagagtgagactccatctcaaaaaaaaaaagctgcctagctgtaacattaaggcattcttttgggagaggtggaggcagagccatttattggttgcatgagaccgttggaggttaacgttgagtaagaatgctgagtggcggtgatggggtaggtaaaggctttagtgtccaggtgaccttaggaggtaagctactaggtggagggaggctgggaaaactaacctggctcactagtcagtttcacaaatgtggcaaaagtggggcttggaacagggtggctgtgggcagctagctgcttttaagacactgaataacctatcaagtagactttgtgtttcttcaaagccttttttttttcttctttttttaaagtaggcctcctaaaatgcacttaaagatgtcaagttagaggtgtaggccttagcttttgtcttcactgacttagtgctagtcgggatgccagactctaactgcgtctagtagcttctcatgacaacactgaggccccactgccagaatgttcctagttgaggatgggactgagtttagagcctcaggtgcatctgatgaaattaaagttgtagtattggtttaattacagaaaccatataattggaaccatgtgctaattatgcctccaattctctaacaaacatcccacttaagtgaaccccttactaccttaggccataggactaagtcttaacatcttggacacttgttaaaagggaccaaagtgagtttgaggcctccatagaatccgtatctcaaggggaaaggcccacctgcatcaatgtggacagagatggtgcgtgtaaaatgcagattactgggcacctgtcctacctacatttaataggcgccccagctgttgctaatagaattgaagcatgagcacctgtgctcggtagagaagaggtggcatctcatggagcttccagtagagtgggcagatggccactagtttttatataaatgtaaaactgaggttctgacctgtgatctgcttatgtgcctctgcagtaagggacctgacttagggagatcagggaagccctgcttaaaagaacgatacctcagctaagctccagttcaaatgagtcgaatgaggcaaaaatggagaggggaaaatttccagattaggttaacaacaatctgcaaaagccctgtggtagaaaacaaccagggggaggccacagtgtgagaatactttggaggacatgacatgccaggtgaatctagcagcgccagactggaagggtgttttaggccttcattaggaggatggtctctactggagtgcaggggctactcacaggcattatcagggaacaccatggcctcaagctcctgggctcaagtgatccatcctcctggctcagcctctcaagtggctgggactacaggcactttgccaggctgcactaaaaggttttaaaggtatggtggggatagtggggcatggtggcatatagattaaaaaatcactccagctgtacagtggatacctgtagataaggagactcacagggacctgataaagtg

SEQ ID NO: 14 - DNA, 1118 bp, human
ggggggagctgggacctaagccgcgcgcacacccctttctctgcgtctggtggaggtgcacagaggcttttgagtcaggcccaagcgcagccaggtgacctccccgcggcctttcaagcctgagctcggtggacagctccctctcccgtgagtcccgctgtcctgtacgcgcccggtcgagccccgggctgcgcaccccgctaggagctgcccggccagcccgcttctctgcccggagccatgaatctcagtagcgccagtagcacggaggaaaaggcagtgacgaccgtgctctggggctgcgagctcagtcaggagaggcggacttggaccttcagaccccagctggaggggaagcagagctgcaggctgttgcttcatacgatttgcttgggggagaaagccaaagaggagatgcatcgcgtggagatcctgcccccagcaaaccaggaggacaagaagatgcagccggtcaccattgcctcactccaggcctcagtcctccccatggtctccatggtaggagtgcagctttctcccccagttactttccagctccgggctggctcaggacccgtgttcctcagtggccaggaacgttatgaagcatcagacctaacctgggaggaggaggaggaagaagaaggggaggaggaggaagaggaagaggaagatgatgaggatgaggatgcagatatatctctggaggagcaaagccctgtcaaacaagtcaaaaggctggtgccccagaagcaggcgagcgtggctaagaaaaaaaagctggaaaaagaagaagaggaaataagagccagcgttagagacaagagccctgtgaaaaaggccaaagccacagccagagccaagaagccaggattcaagaaatgaggagccacgccttggggggcacggtgcaaagtgggccttccctgggctgtgctgcaggcacagggtgcccctgtccagcccctccacctgtgtctgaatgcaacaggggtgttgcgggggcaacatgagagcccctcacccccaactctccactttcaggaggcccccagtgaagagccccacctcggggtcacaataaagttgcctggtcaggaaaaaaaaaaaaaaaaaaa

SEQ ID NO: 15 - protein, 200 aa, xeniopus laevis
MASTVSNTSKLEKPVSLIWGCELNEQDKTFEFKVEDDEEKCEHQLALRTVCLGDKAKDEFNIVEIVTQEEGAEKSVPIATLKPSILPMATMVGIELTPPVTFRLKAGSGPLYISGQHVAMEEDYSWAEEEDEGEAEGEEEEEEEEDQESPPKAVKRPAATKKAGQAKKKKLDKEDESSEEDSPTKKGKGAGRGRKPAAKK

SEQ ID NO: 16 - protein, 424 aa, Human
MAALGDEVLDGYVFPACPPCSYRYPYPAATKGKGAAGGSWQQRGRGCLPASSPCSAGAASLSFPGCGRLTAAEYFDSYQRERLMALLAQVGPGLGPRARRAGSCDVAVQVSPRIDAAVQCSLGRRTLQRRARDPESPAGPGAEGTTGGGSFSQQPSRRGLEQGSPQNGAPRPMRFPRTVAVYSPLALRRLTAFLEGPGPAAGEQRSGASDGERGPPPARLQGPEEGEVWTKKAPRRPQSDDDGEAQAAVRASWEQPADGPELPPREAQEGEAAPRSALRSPGQPPSAGRARDGGDGREAAVAGEGPSPRSPELGKERLRFQFLEQKYGYYHCKDCNIRWESAYVWCVQGTNKVYFKQFCRTCQKSYNPYRVEDITCQSCKQTRCSCPVKLRHVDPKRPHRQDLCGRCKGKRLSCDSTFSFKYII

SEQ ID NO: 17 - DNA, 27 bp, Mouse
gcaaagaagccagtgaccaagaaatga

SEQ ID NO: 18 - DNA, 27 bp, mouse
cctgatcatgcaaattttattgtggcc

SEQ ID NO: 19 - protein, 18 aa, Mouse
KRPHRQDLCGRCKDKRLS

SEQ ID NO: 20 - DNA, 24 bp, Mouse
ctagaaaaggggactgtagtcact

SEQ ID NO: 21 - DNA, 24 bp, Mouse
tgcatctcccacacaagtcttgcc

SEQ ID NO: 22 - DNA, 24 bp, Mouse
ctagaaaaggggactataggcacc

SEQ ID NO: 23 - DNA, 24 bp, Mouse
tgcatctctcacacaagtgttgct

SEQ ID NO: 24 - DNA, 20 bp, Human
ggaggtgtggacgaagaagg

SEQ ID NO: 25 - DNA, 22 bp, Human
aagctgaaggtgctgtcgcagg

SEQ ID NO: 26 - DNA, 26 bp, Human
tgaaggtcggagtcaacggatttggt

SEQ ID NO: 27 - DNA, 24 bp, Human
catgtgggccatgaggtccaccac

SEQ ID NO: 28 - DNA, 1260 bp, Mouse
tgggcgggcgaggcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgacagccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccgatcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgcgaggaaagcggtcccccagccgcgaagcgaggagggcgatgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctcgagaccaggcctccccgcaaagcacggagcaggacaaggagcgcctgcgtttccagttcttagagcagaagtacggctactatcactgcaaggactgcaaaatccggtgggagagcgcctatgtgtggtgtgtgcagggcaccagtaaggtgtacttcaaacagttctgccgagtgtgtgagaaatcctacaacccttacagagtggaggacatcacctgtcaaagttgtaaaagaactagatgtgcctgcccagtcagacttcgccacgtggaccctaaacgcccccatcggcaagacttgtgtgggagatgcaaggacaaacgcctgtcctgcgacagcaccttcagcttcaaatacatcatttagtgagagtcgaaaacgtttctgctagatggggctaatggaatggacaagtgagctttctcccctcttcacctcttccctttccaaattcttcatgacagacagtgttacttggatataaagcctgtgaataaaaggtattgcaaacaa

SEQ ID NO: 29 - protein, 361 aa, Mouse
MFPASTFHPCPHPYPQATKAGDGWRFGARGCRPAPPSFLPGYRQLMAAEYVDSHQRAQLMALLSRMGPRSVSSRDAAVQVNPRRDASVQCSLGRRTLQPAGCRASPDARSGSCQPRGHAGAGRSPRSWQTVAPFSSVTFCGLSSSLEVAGGRQTPTKGEGSPASSGTREPEPREVAARKAVPQPRSEEGDVQAAGQAGWEQQPPPEDRNSVAAMQSEPGSEEPCPAAEMAQDPGDSDAPRDQASPQSTEQDKERLRFQFLEQKYGYYHCKDCKIRWESAYVWCVQGTSKVYFKQFCRVCEKSYNPYRVEDITCQSCKRTRCACPVRLRHVDPKRPHRQDLCGRCKDKRLSCDSTFSFKYII

SEQ ID NO: 30 - DNA, 1275 bp, Human
atggcggccctgggggacgaggtgctggacggttacgtgttcccggcgtgccccccctgctcgtaccggtacccataccccgcggccaccaagggcaagggcgcggcgggcggcagctggcagcagcgcggcaggggctgccttcccgcctcctccccctgctcggcgggcgcggcctcgttgtccttcccgggctgcgggcggctgacggccgccgagtacttcgacagctaccagcgggagcggctcatggctctcctggcgcaggtggggccgggtctcgggccgcgcgcccgcagggccggcagctgcgacgtggcggtgcaggtgagcccgcgcatcgacgccgcggtacagtgctcgctggggaggcgcacgctgcagcgccgggcccgcgaccccgagtccccggccggccccggggccgagggcaccacgggtggcggctctttctcccagcagccatcccgtcgaggcctggagcagggcagcccccagaacggcgccccgcggcccatgcgcttcccgcgcaccgtcgccgtgtactcgcccctggccttgcgccgtctcaccgccttcctggaggggcccgggcccgcggcgggcgagcagaggtccggggcgtcggacggagagagggggccgccgcccgcgcggcttcaaggcccagaggagggggaggtgtggacgaagaaggcgccccggcggccgcagtccgacgacgacggcgaggcccaggccgcagtccgagcgagctgggagcagccggccgacggtcccgagctgccgccgcgagaggcccaggagggcgaggcggctccgcggtcggcgctaaggagcccggggcaacctccgtcggcggggagggcccgagacggcggcgacggacgggaggcggccgtcgcgggagaggggccgtcgccacggagcccggagctgggcaaggagcggctgcgcttccagttcttagagcagaaatatggctattaccactgcaaggactgcaacatccgctgggagagtgcttatgtgtggtgtgtacagggaactaacaaggtttacttcaaacagttttgcagaacttgtcagaagtcttataacccttaccgagtggaggatatcacctgtcaaagttgtaaacaaacgagatgttcctgcccagtaaaacttcgccacgtggaccctaaacggccccaccgtcaagatttgtgcggtagatgcaaaggcaaacgcctgtcctgtgacagcactttcagcttcaaatacatcatttag

SEQ ID NO: 31 - DNA, 1052 bp, Xenopus Laevis
ttcggcacgaggtggatcgctacatgtacccagcttacaatccttattcgtataggtacctgaaccctaggaataaagggatgagctggagacagaagaactatttggccagttatggagacactggggactactgtgataattatcagagggctcagctgaaggccatcttgtctcaagtgaaccctaacctcacgccaaggctctgcagagcaaacacaagggatgtgggggtgcaggtaaaccccaggcaagatgcatcagtccagtgctccttggggcccagaactttactcagaaggagacctggggccctacggaagcctccaccagagcaagggagtcctgcctcccccacaaagactgtgagattccccaggactattgcggtatattcacctgtggctgcaggaaggttggctccatttcaggatgaaggggtcaatctggaagagaagggtgaggcagtgagaagtgaaggctctgaaggagggaggcaggaaggaaagcaaggggatggagagatcaaggaacagatgaagatggacaagacagatgaagaggaagcagcccctgctcagacaaggccaaagttccagttcctggagcagaagtacggatattatcactgtaaggactgcaacatccgctgggagagcgcctacgtgtggtgtgtgcaggaaaccaataaggtgtacttcaagcagttctgcaggacatgtcagaaatcctataatccctaccgtgtggaagacatcatgtgtcagagctgcaagcagacgagatgcgcgtgtcctgtcaaactgcgtcacgttgaccccaagaggccccaccgccaggatctgtgtgggagatgcaaaggcaaacggctctcgtgtgacagcacttttagcttcaagtatatcatttgattgtgtgtaattcatacatttctagctacattaattaagccaggaaagggcttcatttatgtttttgttttggaggagacgactgggaaggaactgtctgtgcaatgcgctcgctttcttgtactgaataaacagaataga

SEQ ID NO: 32 - protein, 295 aa, Xenopus Laevis
MYPAYNPYSYRYLNPRNKGMSWRQKNYLASYGDTGDYCDNYQRAQLKAILSQVNPNLTPRLCRANTRDVGVQVNPRQDASVQCSLGPRTLLRRRPGALRKPPPEQGSPASPTKTVRFPRTIAVYSPVAAGRLAPFQDEGVNLEEKGEAVRSEGSEGGRQEGKQGDGEIKEQMKMDKTDEEEAAPAQTRPKFQFLEQKYGYYHCKDCNIRWESAYVWCVQETNKVYFKQFCRTCQKSYNPYRVEDIMCQSCKQTRCACPVKLRHVDPKRPHRQDLCGRCKGKRLSCDSTFSFKYII

SEQ ID NO: 33 - DNA, 1192 bp, Danio rerio
  misc_feature (1)..(1192): n equals unknown
tcagctatagggcaaggcagtggtattcatcgcagagtttcgcgggggggctaagcgaaaaatggctacatatggaaacgagacagtcgataactatctttactcctcttacaacccttattactacaaataccccaaattcaagggctggagacagaaagcttacttcaccaactacggtgagggcgagacctactttgataatcaccacagggcgcagctgaagtccatcttgtctcagatcaacccaaatctcaccccgcgtctgaggaaggccaacaccaaagacgtcggggttcaggtcaacccgaagaccgacgcgtctatccagtgctctctggggccgcggacgcttctggcacgcaaacgcgatgccctgcgccggcggcggcaggaggtccagacccctgggagtcccgtcagcagcggtggtgtccggttcccgcgcacccaggccgtttactccccagtagaatcccggagactagtgtccctcttcagagaggagggtgaagaagaggaggacacggatctcgaggtcacagagacggttgacagcgcagagaagctggaaagcgccgagaaaaacgtgcgcaaacagggtaagaaaagcgcgaagcaaccgcttagtccagagaaaaatataaacaagcagactgaaacaaatgaggagaacacaaacgagccagtgaaaaccgaacaagacgatctgaagtccaaggctcgtgtgagatttcagtctttggagcagaagtatggattctatcattgcaaagactgcaacctacggtgggaaagtgcttatgtgtggtgtgtccaaggaacaaacaaggtttatttcaagcagttctgcagaacatgccagaaatcattcaacccataccgggttgaggacatagcatgtcagacttgcaagaaagctcgctgcacatgttctgtcaagtcgcgtcacgtggaccccaaaagaccccatcggcaggatctgtgcggccgctgtaaaggcaagcgtctgtcctgcgacagcacgttcagcttcaagtacatcatctagcagctctgtccattgttgttcatcagctggatttgtccacctcagtctctgcancctctgctgctccttcgaccgcagttggggggagggcaggcccattcggcccacttacagtctcattttgttttttgttttgccttt

SEQ ID NO: 34 - protein, 329 aa, Dario rerio
  MISC_FEATURE (1)..(329): x equals unknown
MAXYGNETVDNYLYSSYNPYYYKYPKFKGWRQKAYFTNYGEGETYFDNHHRAQLKSILSQINPNLTPRLRKANTKDVGVQVNPKTDASIQCSLGPRTLLARKRDALRRRRQEVQTPGSPVSSGGVRFPRTQAVYSPVESRRLVSLFREEGEEEEDTDLEVTETVDSAEKLESAEKNVRKQGKKSAKQPLSPEKNINKQTETNEENTNEPVKTEQDDLKSKARVRFQSLEQKYGFYHCKDCNLRWESAYVWCVQGTNKVYFKQFCRTCQKSFNPYRVEDIACQTCKKARCTCSVKSRHVDPKRPHRQDLCGRCKGKRLSCDSTFSFKYII

SEQ ID NO: 35 - DNA, 963 bp, Fugu rubripes
atggcaacgtattgtgacgagccagtcgacagctacttctattcatcttacaacccgtatatgggccggtacccccggcacagagacgccgggtggaaatataaaagttacctctcccactatggggacacttcagaagccttcagtaaccagcagcgggcccagctcaagtccatcttatcccaaatcaatcccaaactcaccccgaggctcaggaaggccaacaccaaagacgtggcggtgcaggtgaacccgaagcgggacgcctcggtgcagtgctccatcggcccgcggaccctcctggtggtgaaacgagaactccggcgcaggagaaaactgaacccgggcccgcccggaactccccagaagacggagggcgaggtgcgctacccgcggaccctcgccgtgtattcgcccattgccttcaggagcgtcacctccttcctggtggagaccgggaaggaccgtcccgccgccgaggcccaggccgaggagctgcccggtgagcagccggagcaaaagggcggcgagaaccaggccggcgaggaaacgaacgcaaatctacccgaacagcgcaaaccgcaaagtgaagatgcgcaaaccgccgcagacgctgaggggtcaaagggcaaagcgcgtgtccgcttccagtttctggaacagaagtacggctactatcactgcagagaatgcaacctgcgatgggagagcgcgtacgtttggtgcgttcagggcactaacaaggtttacttcaagcagttctgtaggaaatgccaaaaagactttaacccgtaccgcgtagaggacatcacatgtcacgtatgcaacaaggcccgctgtgcctgcgcagaaacgcagcgccacgttgacccaaagaggccccacaggcaggacctgtgcggcaggtgcaagggcaagcggctgtcctgcgacagcaccttcagcttcaaatacatcgtctaa

SEQ ID NO: 36 - protein, 320 aa, Fugu rubripes
MATYCDEPVDSYFYSSYNPYMGRYPRHRDAGWKYKSYLSHYGDTSEAFSNQQRAQLKSILSQINPKLTPRLRKANTKDVAVQVNPKRDASVQCSIGPRTLLVVKRELRRRRKLNPGPPGTPQKTEGEVRYPRTLAVYSPIAFRSVTSFLVETGKDRPAAEAQAEELPGEQPEQKGGENQAGEETNANLPEQRKPQSEDAQTAADAEGSKGKARVRFQFLEQKYGYYHCRECNLRWESAYVWCVQGTNKVYFKQFCRKCQKDFNPYRVEDITCHVCNKARCACAETQRHVDPKRPHRQDLCGRCKGKRLSCDSTFSFKYIV

SEQ ID NO: 37 - DNA, 1280 bp, RATTUS NORVEGICUS
tgggccggcgaggcgcgggacgcacccatgttcccggcgagcacgccccacccatgcccgcatccttacccgcccacggcagccaaagccggggatggctggaggtttggagccaggggctgcaggcccgagcccccctccttcctccccggctacagacagctcatggccgcggagtactttgacagctatcagcgagcgcagctcatggccttgctgtcgcgaatgggtccccggccggtcagcagccgcgacgctgcggtgcaggtgaacccgcgccgcgatgcctcggtgcagtgttcgctcgggcgccgcacactgcagcctggacggcgccgagccagccccgacgcccggcctggttcctgccaaccccgcagccccgccagggccgggagacccccgcgatcctggcgcaccgtcgccctgtactcgcccgtgaccttcggtggcctctcctcctcgctggaggttgcgggggacaggcagacgcccacgaagggagaggggagaccggcacccacggggacccgggaacccgagccgggagaggtggcagtgatgaaagcagtcccccagccgcagagcgaggagggcgacgtccaggctgaagggcaggatgggcaggagcagccaccgcgggaggacccggacagtgtggcggcgatgcagtctgagcccgggagtgaggagccacctcctgctgtcgagatggctcaggaccccagtgacgtggctgcctctagagaccgggcctccccacagagcactgagcaggacaaggagcgcctgcgtttccagttcttagagcagaagtacggctactatcactgcaaagactgcaacatccggtgggagagcgcctatgtgtggtgtgtgcagggcaccagcaaggtgtacttcaaacagttctgccgcgtgtgtgagaagtcctacaacccataccgagtggaggatatcacctgccaaagttgtaaaaggactagatgtgcctgcccggtcagacttcgccacgtggaccctaaacgcccccatcgtcaagacttgtgtgggagatgcaaggacaaacgcctgtcctgtgacagcaccttcagcttcaaatatatcatttagagagagttaaaaatggttctgctaaatggatcggacaagtgagccttctcccacgcccctctcccctctccacctctcccttctcaaaatacttcatgaaaggcagtgtactttaatataaagcctgcgaataaaaggggttgcaaacagtttggggctttatccaa

SEQ ID NO: 38 - DNA, 3959 bp, Rattus norvegicus
ctgggcggcgctgatggcagcctgggccggcgaggcgcgggacgcacccatgttcccggcgagcacgccccacccatgcccgcatccttacccgcccacggcagccaaagccggggatggctggaggtttggagccaggggctgcaggcccgagcccccctccttcctccccggctacagacagctcatggccgcggagtactttgacagctatcagcgagcgcagctcatggccttgctgtcgcgaatgggtccccggccggtcagcagccgcgacgctgcggtgcaggtgaacccgcgccgcgatgcctcggtgcagtgttcgctcgggcgccgcacactgcagcctggacggcgccgagccagccccgacgcccggcctggttcctgccaaccccgcagccccgccagggccgggagacccccgcgatcctggcgcaccgtcgccctgtactcgcccgtgaccttcggtggcctctcctcctcgctggaggttgcgggggacaggcagacgcccacgaagggagaggggagaccggcacccacggggacccgggaacccgagccgggagaggtggcagtgatgaaagcagtcccccagccgcagagcgaggagggcgacgtccaggctgaagggcaggatgggcaggagcagccaccgcgggaggacccggacagtgtggcggcgatgcagtctgagcccgggagtgaggagccacctcctgctgtcgagatggctcaggaccccagtgacgtggctgcctctagagaccgggcctccccacagagcactgagcaggacaaggagcgcctgcgtttccaggtgaggccagcctggcaacctggacgcttccagaattgtaggactccttcggggctaagctagtggttgtggctgatgcagggcatagaattcttcaatgccctcagtctgtatttaaaaaaaaaccccaaccgcgtatgggtgttttgattgcatgtagtgtctgtgcatttcgttggagtggagagatgggagcggaagagggcgtcagatccccggaactggcgatttacaggtggttgtgagccactatgtgggtttgaggaactgaattagggtcctctagaagaactgtgttctttaacagctgaaccatctctccagctcttcagcttgtaggagcttgcagccgctgccagttaatacaatgggaggtgtttacacaataaaaccaacccctacgtggcctgacccactggcagtctctgttcttgggaaatgactgtgtagttgattctggtcacatcaacactacaaatgaagcctccgcggcgaacgcacaccagtccagtttggaccgtacttcagatggctgaaggaagataagaaaacacacagcagaatatcatcatgtgaacttggtcgcacgagagagtgggtttcccaatgctccttttctcatttcccctctagatatggggaaatttgcctcatttgactggtggggttttgttctcaactaagccctaccctccatttttgaagctctcaactgaagcttctccctcccaaacttgcctggcttttaaaaaaacaaaaaaacaaaaaaacctcagcattcatccagaaggccagcactcctaagccttggatggtcttgagttgcagcctaggctaaccaaacaaggaactggggggcgggggtgggggcacccaaggagcgtggccattactccctccccccaaagagatgcagagctctgagctctgggaaaggactcagaaatgccttattttctaatcatttcaaggttgttgtgtttggggttggagtggcagtggaatggggtgtcagaaaagcacttggttgccgttcacagctgggggtgatcttaggcagaaaccttaagctttctggcctcctgtgtctgctcactataaaaattgcattaagagtccttccaggcccccctgccagtcctaaaggcttaattataagagcttgtttgtcacttaagtgggaagcggggaagtttagaactcgggtgctaacgttacatagtcataccaaatgagctatacttacataggtggcgggactttttggaaacttaactagcatcatggactatttaacattgattgaagactttcgaaggtttagaagagcctttctctttcagtgtaataactctctcatctgtggaagttctaggatcactcatcaccccacataaaaggcccaagggaaggatcctgagctctagacagcactatttggcttttcccttgtccgaggggaaaaacacttatccatctgctggtataggaactttcttggttggcctcagtttacatatttgtttgactgaactattatttcacctgattaatttagaggttgacccaaatctaaccttacttactaccatggtaaagtttcaagtatcaggttgaatctggtcccagattctgaaatgagggtaactcagccatcacttgcttaaagtctgtatcctgctgttcagttcttagagcagaagtacggctactatcactgcaaagactgcaacatccggtgggagagcgcctatgtgtggtgtgtgcagggcaccagcaaggtaagagactctgcggctgtcccattcctgcctggtgccgagtggggagtgtcagtgtcatggggaccctctttctcatcgcaggtgtacttcaaacagttctgccgcgtgtgtgagaagtcctacaacccataccgagtggaggatatcacctgccaagtaaatcaaatgtttgcattttggaaaaggggtttcgtgtgctatttcgaatatatttcttaaaagaggtgtaggtttccaggagccttaggccctactttttctttcctttttgtttttcgataggggtggtagaaagtcccctctgctgtgaaacggggtgtttgtccccttctgctcttgcaaaatttgactgcatggcctcttttgaatctgttacctcattagcagtgtaaaagacttgcttacctgccacttgacttggcataagggaaaaccctgcaaaagcagagtacctttctatagcctgaggtggtacactattaccccggtgcatctaagaagcacatttccaagttagagtatgagacccaagacatggagcctcagcttttcttgagagtttgcatagggagttagctccctatgagaccttctgagaccttgtctgtccagctctgtgcagttcacccgtaatgaaggctgctaagtgcttgcttaccattaattagttcagcactgcgtcaagggacttagataaccttcaaaaagaaagaagtgacaacagttacctgctaaggttaaccacagagctaaaggacttccacatctacctgagagtccagcgcacttaatgttggtaattggagaattaactaccctaggatgggccctgagaaccctagaatggaaagccacgaatgctccattcagaaaaggggactgcaggcaccgttctacaaacttaaagccttgagcctttctgctcttggctctcggtgcccaattcacgtgccacctgttctgtagtctgctttgaagccccaagaattgagggtgggggaggggggttcatcctttgtacttttcactctgatttttcagagttgtaaaaggactagatgtgcctgcccggtcagacttcgccacgtggaccctaaacgcccccatcgtcaagacttgtgtgggagatgcaaggacaaacgcctgtcctgtgacagcaccttcagcttcaaatatatcatttagagagagttaaaaatggttctgctaaatggatcggacaagtgagccttctcccacgcccctctcccctctccacctctcccttctcaaaatacttcatgaaaggcagtgtactttaatataaagcctgcgaataaaaggggttgcaaacagtttggggctttatccaa

SEQ ID NO: 39 - protein, 361 aa, Rattus norvegicus
MFPASTPHPCPHPYPPTAAKAGDGWRFGARGCRPEPPSFLPGYRQLMAAEYFDSYQRAQLMALLSRMGPRPVSSRDAAVQVNPRRDASVQCSLGRRTLQPGRRRASPDARPGSCQPRSPARAGRPPRSWRTVALYSPVTFGGLSSSLEVAGDRQTPTKGEGRPAPTGTREPEPGEVAVMKAVPQPQSEEGDVQAEGQDGQEQPPREDPDSVAAMQSEPGSEEPPPAVEMAQDPSDVAASRDRASPQSTEQDKERLRFQFLEQKYGYYHCKDCNIRWESAYVWCVQGTSKVYFKQFCRVCEKSYNPYRVEDITCQSCKRTRCACPVRLRHVDPKRPHRQDLCGRCKDKRLSCDSTFSFKYII

SEQ ID NO: 40 - DNA, 6873 bp, Mouse
ggatccctgttgcagtcataccctatgggaaaagagcaacttacctatcttaaggagattgggggaatttagatatttgtgcatcctctttcacttgaatgagaacaatgtaccagatcgtcaacagtgcacatttgacccggccagtagcaacatacctaaaactacctcaccattgtaaaacaccctaagaatcacaagaaacttacagtttttcagagacaaatcaaggagcagagttacagaattaaaaacaattcatcataccttagaattttctctaatcgaatgagttgataattgtttccatactcttagccatctttggagctatactttgaaggttaaaaatatcctagactaagttagttttcattgtaagtgctagcagcctttgctttctggtgtgaaataagaactaatttcaagtagaaagcacagagttcaggagagatgaagactatgttgcccaggctagtcctcagcccataactgggttcttgggctaaattctagctgctccattggaaataagcaagacagtgaattaagcacacacgaagagtactaaacgttgcgtggccaaggccgagaggctgagggtctgcctagtaacggtaacctttccctgatctctggtttatcgttttgaagaccttttcagaaggagtagtctgtctgtatgtccttccatcccaagcaagtgaagagcccagaggagccactggatatcaagctaggtctccaagcagttatgtctaaggagcttcaggctttgtttgcgaggttagaatggatgtagcttgtccagatgcctcctcagtgatgttgttgttgataggttgcccatgtgtttcttttcttaccaggtttccctgtggtctctgcttgtgaccctggtggttcttttcataatgactggcaccatgctgggacctgaactgctggcaagcattcccacaactgtttacgtggtcgccatttttatgcctctggcggctacgcctcgggttatggcttagctaccctcttcacctccgcccaactgcaagaggactgtgtgtctggaaacaggaagtcagaacgtgcagcttctgcactgcgattcttaaacttcccgcctcgctttataggtagcatgtacatgttcccttctgctctacgccttcttccagtctgccgaggcaggggtcttcgtgttgatctacaaaatgtacggaagtgagatactgcacaagcgagaggccctagacgaagacgaagacaccgatatttcttataagaaactgaaagaagaggaaatggcagacacctcctatggcaccgtggggacgcatgacttagtgatgatggagaccacccagaccgccctctgactgaggagatacacgggagctgaaacatcacttcctatttgtgaccattggtagcgagtatggttcgcatccgggataaagatgggttgacatttcctgtaacagatttgctcttcccactgtaatgtagtatctcagtattacccatgtgtttttctaactcaacagagtgtcccaatattgcttacacctggatcagctaaagtgccgcgtcctctgcttaagtagtgtgctgtttgtttgtttgttttttgttttgttttgtttttttccatttccaccagcattgctacagataggaaatggggttggaaatgtttgtaaaacagaaccatgggtttgttcaacttacaaacaaccgattctgttcagggcgagcctgtattgagaaaagtccaaaacgggtcaaaaagggttgaaacgacaggatagcattgcatcgtcaagccagagaaaaccgtattaatgtgtgtgactacttgatctagtatctattgttaatggccatcaacattgtgcaggggtgaaaggcatttttccccatatgtttcctgtatgtgtataaacgcatctcagctccatttatcgtctgaaggaatgatttacttaggaaaatgcgtagacctcacctcagggagagaaaatgggccactttgttcatccgtgggaaagggctgtggctacaggctttccttccggaaaggcctgtggctggacactgtcccactgctctggtagactggagctgtgatctgagacaacctaagaggttcagagcagtctcctaaccttggtattttgctccctaatcagacacactggcctcccttgtcttcttcatgacagacatctggagctacagacatgggggcccacctggctcggctaatctcggtgatgattctggggttgaattctcatctcatctagttcccctacaaatccttgctgtggctagcaaggaaagctctttttctgcatccacgagggagtgggggtgggggtcgcctcttaaccagtgtggggaaggttttgctcctcatggcaacagcaggtggtagggctttttctaccagtgcgcggccgcctatttaacgcagcgtggagggcagctgggctgcgctgatggctgcctgtgggcgggcgaggcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgacagccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccgatcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgcgaggaaagcggtcccccagccgcgaagcgaggagggcgatgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctcgagaccaggcctccccgcaaagcacggagcaggacaaggagcgcctgcgtttccaggtgaggccagcctgatggcctggacgcctccagaattgtagggctccttcagggctaagctggtggctctgggtgatgcagaacatagaattcttccatgccatccgtctggttttgtttgtttgtttgtttgtaacatgtttggtgttttgattgcatgttgtatctgtacacttcgttgtagtggagagatgggagcagaagagggtgtcggatccggatcccctgggactggcgttttacagatggttgtgagtcaccatgtgagttttaggatcggaattacggtcctctagaagaacagggtgttgtttcacagctgagccatctctccagctctttggcatataggattttgcagccgctgcctgttaatacaatgggaggcgtttacacaataaaaaccaacccatatgtgtcctgacccactggcagcctctgctcctggggaatgccagttgtaattattctgatcacataaacgctacacatgaggtctccgcggagaatgcgcacagtctgggtttggaccaaacttcagatggctgaaggaagataagtgcacacatggcagaaacataatcttttgaacttcgttgcggggagagtcggtttcccaaggctcctttttttatttcccctctagatgatctgtcttggttaacttgccggcttgttctataccagccccttcccttcgtttctgaagctgtcaactgaagcttctctctcccaaacttgcctggcttaaaaaacaaacaaacaaaaacaaaacaccccccccaaaaaaaaaaacaacaaaaaaaaaaaagaaaagaaaaagaaaaagaaataaaagaaaaaaaaaaccactctccccattcatcgaggccagccactgctaagctgttggatggtcttgagttgctgcctgtgctagcaaacaaggaggcacaaagagtgctgtaggtcgtatacccccaccaaagaaatggagagccctgagctccaggagaggactctgagacattccttgtttttcagtcatttcaaggctggtgtgtttgaggttggggtggcagtggaatggggtgtcagaaaaaatagaaaagtgcttggcggttgctgttcacagctgggtgtgatctcttaggcagaaatcccaagttttcgggcctctgtggtggtcgttcacctataaaaaattgcattaagagttcttccaagccctgccactcctaaagacttagttataaaaacttgtttccaacttgtttgtcactaagtgggaagcttgggaagtttaagaaccaggtgctaacactatgtagttcataccaaatgagctagacttgggtaggtagcgggactcttttggaaacttacctagcatcaaggaaaatttagtattggttgaagactttcaaaggttttagaagagcctttctctttcggcataacaactttcccatgtgtgagtgtcctaatgcatcgcccacataaaatgccacgggaagaatcccaaactctaaaccgcacgatttggcttctcccttgtctgaggggggaaaaaccacttatcggtctgctgctatatgaactatcttgtttggcctccgtttacatatttgtttgattgagctattagttcacctggttaacttagaggttgacccaagtctaaccttactaccacggtaatcttaaagtatcaagtggaatgtggtcccaggttctgaaaattagggtcactcgggcatcacttgcttaaagtctggtaccctgctgttcagttcttagagcagaagtacggctactatcactgcaaggactgcaaaatccggtgggagagcgcctatgtgtggtgtgtgcagggcaccagtaaggtaagagacaccgtgcagccctcctgctctgctgtgttgccgagtgtctgctccatgccgatgtctttctcctcgcaggtgtacttcaaacagttctgccgagtgtgtgagaaatcctacaacccttacagagtggaggacatcacctgtcaagtaaaccaaacgtttgcattttggaagaggggtttggtgcacgactttgagtatatttcctgaaggaggtggtttccagtagctttaggctctaccttttccctcctccttccttttcatttttgactaggttggtggtagaaagtcccctccactgtaaatggggtgtttactcccttctgctgttgtaaaacttgattgcatgccctctcttgcatctggttaccttgttagcagtagaaagggcttgcttacctggcttcttcccactcggacctaagggaaaacatattgcaaaacagagtgcctttctgctagcttgagatggtacacattaccccaatgctacataggaaacacattcccaagttagcatatgaaacacaagaaattgagctctggcttttcttgagagtttacaaagggagtttcctgtaagaccatcctacactgtctagctctatgcagtttacccataactgtggctaagagtttgcttgcttagtattaatttagcactgtgccaagggacttagataaccttgaaaacatttacctgttaaaattaatgacagagataaaggaattcgaattccacatctgagagcccagtgcacttaaagttggtaattggagaattaattaccttagggtgggccctgtgaaaccgagaatggaaagccactaaagactccatctagaaaaggggactgtagtcacttttctacaataaggggccttaaacttccctaagcttccctgcacttggttctcagtgcccagcacacaggccacttgttctgtaatctgttttgaagctccaagaatcgagtggagacagggctcaccctttgtacttttcactccgatttttcagagttgtaaaagaactagatgtgcctgcccagtcagacttcgccacgtggaccctaaacgcccccatcggcaagacttgtgtgggagatgcaaggacaaacgcctgtcctgcgacagcaccttcagcttcaaatacatcatttagtgagagtcgaaaacgtttctgctagatggggctaatggaatggacaagtgagctttctcccctcttcacctcttccctttccaaattcttcatgacagacagtgtacttggatataaagcctgtgaataaaaggtattgcaaacaagtttgaggctttatccaattcatgtgtcagtttgaggggtgcatgtgcggagagtcaataactttcttaacatttgttgatgagagtgagtcaggctgacttaaggaagttaaaggcacctcattcaacaattaagatttttctttctttttgtttagttttattttatttataaatatatgagtacactgtagctgtcttcagacacaccaaaagaaggcatcagatcccattacagatagttgtgagccaccatgtggttgctgggacttgaactccggacctctggaagagcagttggtaaacccctttcttaactgctgaaccatctctccagcccaaatcttaaggttttacagacaagaatattacagg

SEQ ID NO: 41 - DNA, 6002 bp, HUMAN
tagatgaagatgaagatacagatatttcttataaaaaactaaaagaagaggaaatggcagacacttcctatggcacagtgaaagcagaaaatataataatgatggaaaccgctcagacttctctctaaatgtggagatacacaggagcttctatcttgctgaaatattgcttcatatttatagcctgtggtagtgcacatggttaacataaaagataacactggttcacatcatacatgtaacaattctgatctttttaaggttcactggtgtattaaccaaacgttgtcacaaattacaaatcaatgctgtaatataatttgcacctggaatggctaacgtgaagcctgaattaaatgtggtttttagtttttaccatcaccaatttctatgactgttgcaaatacagaatctattagaaaacagggtcttggaaatgtagaattttggcgcactatgaggaaaaacaagctatctttgtaaagcataattgagtttaatgtaattgttgtaaaaaaaaaagtgtgcttgctctacttaaaattcctcacaatgttgaattttgacctgtattcagaagaattccaaaacaggtcagttaaataaggaaatatagtatttgtcaaaccagtatcagagaaaagttacattaatgtatttgattacttgatctggtatctacttattaatgaataatcaacatttttctagtggacaagccatgttcttttcgactggtcttctgcatttatgtaaacacaccccaaatcaatttatcttttgtaggaatgatttggttgggaaatttttcagaaccgctctggagcagaacaaaacggtacctcccggtcaccactgggactgggggaagggacacgctccaggggagaaaacaattcgcctagatgaagatccctgggtggttctccatgcactcgccgagggggctcagtgggtagcctcttagagcagctctgagaataaacttatcatctgagttaacgggtaatagacccagaacagttcccaaaccttggcactttcgctcacttagccagaggcacccggcctggcctcggcttcccggtgagggagcgggggtgggggggatgcgcaggcacctggaacaatcagggcaccgggagaagccggctcagctgatgccggtgatgagtttctctcattgaaatcctcctcacctcgggcgccttggttcccttacggatcagccctttcatcacaaagaaagccctctttccagatcatctaagggtcattgtgccaacatccgggcgtggagagtttcygtagggagaaggacgaagaggggccccctcggcggggacgcgggccggtggcaggaagggcgtggagggcggtgcagcgtgcgagcccccgccgagggccatccccgcctccgctcggccgcccgggcaagtcgcctatttagggtgcggcggcgggcgggagcagtgcgcccatggcggccctgggggacgaggtgctggacggttacgtgttcccggcgtgccccccctgctcgtaccggtacccataccccgcggccaccaagggcaagggcgcggcgggcggcagctggcagcagcgcggcaggggctgccttcccgcctcctccccctgctcggcgggcgcggcctcgttgtccttcccgggctgcgggcggctgacggccgccgagtacttcgacagctaccagcgggagcggctcatggctctcctggcgcaggtggggccgggtctcgggccgcgcgcccgcagggccggcagctgcgacgtggcggtgcaggtgagcccgcgcatcgacgccgcggtacagtgctcgctggggaggcgcacgctgcagcgccgggcccgcgaccccgagtccccggccggccccggggccgagggcaccacgggtggcggctctttctcccagcagccatcccgtcgaggcctggagcagggcagcccccagaacggcgccccgcggcccatgcgcttcccgcgcaccgtcgccgtgtactcgcccctggccttgcgccgtctcaccgccttcctggaggggcccgggcccgcggcgggcgagcagaggtccggggcgtcggacggagagagggggccgccgcccgcgcggcttcaaggcccagaggagggggaggtgtggacgaagaaggcgccccggcggccgcagtccgacgacgacggcgaggcccaggccgcagtccgagcgagctgggagcagccggccgacggtcccgagctgccgccgcgagaggcccaggagggcgaggcggctccgcggtcggcgctaaggagcccggggcaacctccgtcggcggggagggcccgagacggcggcgacggacgggaggcggccgtcgcgggagaggggccgtcgccacggagcccggagctgggcaaggagcggctgcgcttccaggtaaagcctagggcggtcagggcacaggggagcccgggggtgcgggtgtcttccttgggcctggccctgtgactgcttcgggcactcggaggtgcggcgcttccctaagcgtgggcyacttccgtatttccgagacagccaatgaccgcgataggtgtcttccttgacagcacagtctcatgtccccgacatccagacttactcgtggcggctgctccacgggctggccagggcgacgcccttgggacgttcttataacccacatatttgcactgtaaacctcgcgcagtgggcgcataggccagccctgaccgcacggttggattacctatcagtaggcacaactgaacttcggagcacttgccggctggagagtcgattcccaaggatccctctctcccatttccgcactggatgtggcaaaaacccttcaactgctgggattctggtcagcaattctgatttctcctttacgagcttccaggctattggaaagctggagctcctaaaatgccccttcctaggaatttgctttgcttttaagaagcacccccaactcagaaatccaatactgcgaaagcatttggactgctcagtgttgctgcccgagggcagcaggctgaaactttaaagggctggggcacgcagagggcagttgtgacctagcagaagtggaaaggcacaagaggtggtaagaagcccgagggagtccctcgcggtctcttccacggccaccacaggctggtattccttttgaggggcggggttggtggcggtaggctgattgtgcgaggagtgaatcgagaggccagggcttcccagcgtggctgtgcaggagctgtgtgtgatcttaggccagtcataaccttcctggacttagcgcagtctcacaggtgagcagactgaattaagtgctccccagagttccttccaattctgaaaggctaactctaaaaacgtgtgcataactgcttgcttactgggagggaagaagggaagtttaagtaacactacttttgttcatattgaatatgaattatggcttacgtacgatttaggttcctggcaccactgtttgggagttaactagcagcatgaagaatgtgatcttgggtgaacctttaaagttccttagatgtggagtcttatttttcttcagcttaatacatgtgtgcatagtctaagatcaggctttatcttaaaaggccttcctacagaatcccaaactttagagaactgtttattatgtccctacttattcgtttattagccagccttatgaactgagttaatctggtatatgaactctaaggcccatgcttgcttaattgtttgactaggctattaagctcacttaattactgtattgaagaggcgtacccaaacctgacctgctttctttcatgatctgaagttgccaatttcaaatatcaagtagatccttcccaacgtctgaaatgaaggataattcaggtgttttgttggttaaattgacatatcctgttgttcagttcttagagcagaaatatggctattaccactgcaaggactgcaacatccgctgggagagtgcttatgtgtggtgtgtacagggaactaacaaggtaagaaataccaggtaactggcatcttcttgctgaaagtgtcaaggcgattttaagtttatcctctttgtcatcacaggtttacttcaaacagttttgcagaacttgtcagaagtcttataacccttaccgagtggaggatatcacctgtcaagtaaatcagatgttttgcattttgtctgacctgggcagtcgtcgagggtttttagtatagtttgagtatacttccaaaaagaggccaggcccccagaccttaggtttcaactggcttttgttaggagtggtagaaacaatactcagctgggaaacggggccttggtgttagcttctttctggccttgcaaatcttgctgttgttaacctcttctaaaactgttaacctcacttgcaatatggaagaatacttgtcttacttgctacttagtctaatgtataagaaaatcaacaaaaacatgcttgtcagctaacatgaggtagtcaaggttgactgttttaccgaaacgcttcttatgaagcacaaccttaaagtacttaagcacagggggttagtttgtcttgcctgaaagctcacaaagggacagtttaagataaatctaagttgtctagctttatggggagttgactataatggtaagcaagcaatatgttaactaagcattgcttaagcgcttgcttgctattaactgtgctaaggggcttagctaatctttaagaggaaagaagtgactacattcgcctcctgtcacacagctaatggagtctgaattgccagttgagacagcctaatcaatacacttgacccacgttggatatttaaaagcattaacaccctggggtggtggagagaaactaagtatggaaagccacttagaatcacttagatcagagctgggcatgtttctaaaagaggatgccttaaccactctgctcttggtgttcattgtcaaattcatccctgacttgttctctaccctttctcttaaacagttgttgtaaaagaaatttcacaattcataattggatctgatgcaatatagcagcagtacagcatggttaaacacccactattcctagccctgtcattgctacgtaggtagggatgtagagggaaaacaagattactatgggaccttgcttagagcacattcattaagtacttgaatggactagaaaaatgttgaagtcctaggaaatcactaagggtttatcttctgcatgcccttctgtatttttttcccccagagttgtaaacaaacgagatgttcctgcccagtaaaacttcgccacgtggaccctaaacggccccaccgtcaagatttgtgcggtagatgcaaaggcaaacgcctgtcctgtgacagcactttcagcttcaaatacatcatttaggtgaaagtcagtgttgctgtgcatgcgctgatggagtagacgagtgagcttttccgtgcctctcctccacctctcccttctcaaaatacttcatgaaaggcagtgtattctgaaaaagccttcaaataaaggtattgcaacacgatttatacattgcataaaatctgtctttgaaaataaagtttcaagagcgcttgtcttgtgctaacagtctgggcctgtcacttcacctttatgaatgcttgctgatggcatagagtgggccaggctctgagttaggctgcagccacttggaaaacaatttaggggggtgcttgtagacgaggtctacttatttaggcaggtctggaggactgaagcttagaaggaagttaactgaataaaaagccgcctagcgatcgcgccactgcactccagcctgggtgacagagtgagactccatctcaaaaaaaaaagctgcctagctgtaacattaaggcattcttttgggagaggtggaggcagagccatttattggttgcatgagaccgttggaggttaacgttgagtaagaatgctgagtggcggtg

SEQ ID NO: 42 - protein, 207 aa, Rat
MSRHSTSSMTETTAKNMLWGSELTQEKQTCTFRAQGERKDSCKLLLSTICLGEKAKEEVNRVEILPQEDRKSPITVATLKASVLPMVTVTGIELSPPVTFRLRAGSGPVFLSGQECYETSDMAWEDDEEEEEEEEEDEDEDEDADLSLEEIPIKQVKRAAPQRPTSIAKRKKVDKEEEAAVRPSPQDKSPWKKGKSTPKPKRSTSKK

SEQ ID NO: 43 - DNA, 624 bp, Rat
atgagtcgccacagcaccagcagcatgactgaaaccacggcaaagaacatgctctggggcagtgagctcactcaggaaaaacagacttgcacctttagagcccaaggcgagaggaaggacagctgtaagcttttgctcagcacgatttgcctgggggagaaggccaaagaggaggtgaatcgtgtggaaatccttccccaggaagacaggaaatcaccaatcactgtcgccacgctgaaggcctctgtcctgcctatggtcacagtgacaggtatagagctttctcctccagtaacttttcgtctcagggctggctcaggacctgtgttcctcagtggccaggaatgttatgagacttcagacatggcctgggaagatgatgaggaagaggaagaggaagaggaagaggatgaagatgaggatgaagatgcagatctatcgctagaagagatccctatcaaacaagtcaaaagggcagctccccagaggccgacgagcatagctaagagaaaaaaggtggacaaagaggaggaggcggcagtgaggcccagccctcaggacaagagcccctggaagaagggaaaatctacacccaaacctaagaggtcgacgtccaagaaatga